SEQUENCE LISTING (4 sequences)

SEQ ID NO: 1 - DNA, 21 bp, Artificial Sequence
  synthetic construct
caggctatttgggttacaccg

SEQ ID NO: 2 - DNA, 20 bp, Artificial Sequence
  synthetic construct
ccatgtgctggtatcgaggg

SEQ ID NO: 3 - DNA, 20 bp, Artificial Sequence
  synthetic construct
tgcgtcctctctcaggagtg

SEQ ID NO: 4 - DNA, 20 bp, Artificial Sequence
  synthetic construct
ggtccatgatgtcgtcagcc

(19) United States
(12) Patent Application Publication
(10) Pub. No.: US 2017/0216360 A1
(43) Pub. Date: Aug. 3, 2017

(54) METHODS AND COMPOSITIONS FOR IMPROVING HOMING OF CELLS INCLUDING MESENCHYMAL STEM CELLS

(71) Applicants: SANOFI, Paris (FR); MASSACHUSETTS GENERAL HOSPITAL, Boston, MA (US); BRIGHAM AND WOMEN'S HOSPITAL, Boston, MA (US)

(72) Inventors: Brigitte Benhamou, Livry-Gargan (FR); Gerald Boquet, Gif-sur-Yvette (FR); Jean-Francois Deleuze, Combs-La-Ville (FR); Jeffrey M. Karp, Brookline, MA (US); Oren Levy, Brookline, MA (US); Charles P. Lin, Boston, MA (US); Luke J. Mortensen, Somerville, MA (US); Christelle Perrault, Villeneuve-Le-Roi (FR); Jidong Zhang, Paris (FR)

(21) Appl. No.: 15/113,259

(22) PCT Filed: Jan. 21, 2015
(86) PCT No.: PCT/US2015/012249

§ 371 Date: Jul. 21, 2016

Related U.S. Application Data
(60) Provisional application No. 61/930,400, filed on Jan. 22, 2014.

(51) Int. Cl. A61K 35/28; A61K 31/496; A61K 31/4545; A61K 31/454; A61K 31/437; A61K 31/404

(52) U.S. Cl. CPC A61K 35/28; A61K 31/437; A61K 31/404; A61K 31/4545; A61K 31/454; A61K 31/496; A61K 2035/122

(57) ABSTRACT

The disclosure relates to methods and compositions for improving homing of cells including mesenchymal stem cells (MSCs). Compositions include compounds described herein as capable of inducing expression by MSCs of cell surface homing ligand molecules such as CD1 la, promoting increased firm adhesion by MSCs in an in vitro shear flow assay, increasing binding to an adhesion molecule such as E-selectin or ICAM-1, and/or demonstrating anti-inflammatory activity upon in vivo systemic administration in cell therapy using human MSCs. Also described are screening methods to identify small molecule compounds for improving a homing function of MSCs.

### CROSS REFERENCE TO RELATED APPLICATION

This application claims the benefit of and priority to U.S. Provisional Patent App. No. 61/930,400, filed Jan. 22, 2014, which is hereby incorporated by reference in entirety.

### STATEMENT REGARDING FEDERALLY SPONSORED RESEARCH OR DEVELOPMENT

This invention was made with governmental support under award No. NIH-P41 EB015903-02S1 and HL095722, each awarded by the National Institutes of Health. The government has certain rights in the invention.

### FIELD OF THE INVENTION

This application, at least in part, relates to compositions and methods in the fields of homing of cells including mesenchymal stem cells (MSCs), cell therapy with MSCs including for disease conditions, injuries, anti-inflammatory indications, autoimmune disorders, and regenerative medicine.

### BACKGROUND OF THE INVENTION

Mesenchymal stem cells (MSCs), also known as mesenchymal stromal cells, are of interest for therapeutic purposes. In the medical area of cell therapy, it is desirable for MSCs to efficiently target or home to disease sites of interest such as an inflammatory site. As an overview, cell homing can be appreciated simply as the delivery or migration in the body of cells to the site of pathology or a desired target tissue, such as in the context of an injury or disease.

Although previous efforts have explored therapeutic use of systemically infused MSCs, studies have often demonstrated poor homing to diseased or damaged tissues. A variety of approaches to modify MSCs has been attempted to yield improved homing in the context of systemic cell therapy. Genetic engineering of MSCs has been used with DNA transfection to introduce or increase expression of homing ligands by the cells. Surface modification techniques have been tried which involve chemically attaching cell adhesion molecules to the cell surface. Another approach utilizes pretreatment of MSCs with cytokines, biological molecules which themselves typically require recombinant gene expression. Despite these various approaches, there remains a need in the art for superior or alternative technologies to provide advances in homing of MSCs. Advances in homing for a variety of cell types is also desired.

### SUMMARY OF THE INVENTION

The present disclosure relates to embodiments of methods and compositions for improving homing of cells including mesenchymal stem cells. In embodiments, the invention provides methods and compositions for improving homing of mesenchymal stem cells. In embodiments, the invention provides methods and compositions for improving a homing function of mesenchymal stem cells. In embodiments, the invention provides methods and compositions for inducing cell surface expression of a homing ligand. In embodiments, the invention provides methods and compositions for inducing cell surface expression of CD11a. In embodiments, the invention provides methods and compositions for improving a cell adhesion ability of mesenchymal stem cells. In embodiments improved MSCs are improved relative to MSCs which are untreated analogous MSCs, negative controls, or in comparison to values respectively thereof.

In embodiments, the MSCs are human MSCs. In embodiments, the MSCs are non-human mammalian MSCs.

In embodiments, the invention provides methods and compositions for use of treating a subject by administering an effective amount of pretreated MSCs, where the pretreated MSCs have an improved homing function relative to untreated MSCs.

The disclosure also relates to embodiments of methods of screening to identify small molecule compounds capable of improving a homing function of MSCs.

In an embodiment the invention provides a compound of a formula as described herein, in any of its stereoisomeric forms or a mixture of stereoisomeric forms in any ratio, or a physiologically acceptable salt thereof, for use in improving homing of MSCs, or in inducing the expression of a homing function, or in inducing cell surface expression of CD11a, or in inducing ICAM-1 binding activity, or in inducing E-selectin binding activity, and/or in the cell therapy of any of the diseases and conditions mentioned herein, for example inflammatory conditions. In embodiments, the diseases and conditions include injuries, anti-inflammatory indications, autoimmune disorders, and opportunities for enhancement of health or function by regenerative medicine. In an embodiment, the condition includes a surgery or a planned surgery, or other medically desirable induced trauma or planned instance thereof.

In an embodiment, the invention provides use of a compound of a formula described herein, in any of its stereoisomeric forms or a mixture of stereoisomeric forms in any ratio, or a physiologically acceptable salt thereof, for the manufacture of a medicament for improving homing of MSCs, or in inducing the expression of a homing function, or in inducing cell surface expression of CD11a, or in inducing ICAM-1 binding activity, or in inducing E-selectin binding activity, and/or in the cell therapy of any of the diseases and conditions mentioned herein, for example inflammatory conditions. In embodiments, the diseases and conditions include injuries, anti-inflammatory indications, autoimmune disorders, and opportunities for enhancement of health or function by regenerative medicine.

In an embodiment, a compound for improving homing of MSCs is not PKC-dependent. In an embodiment, a compound for improving homing of MSCs involves one or more other kinases such as Rsk2, GSK-3β and CDK2.

### SUMMARY OF SEVERAL EMBODIMENTS

In embodiments, the invention provides exemplary aspects, including the following.

### Embodiment 1

A method of treating a subject with a disease or injury condition, the method comprising the steps of:
(a) providing cells in vitro;
(b) contacting the cells with an effective amount of a compound composition comprising the compound having formula I, thereby generating a composition comprising pretreated cells, wherein the compound is capable of improving a homing function in pretreated cells relative to that of untreated cells, wherein said contacting optionally includes incubating the cells with the compound composition; and
(b′) optionally washing the pretreated cells;
(c) administering an effective amount of the composition comprising pretreated cells to the subject;
wherein the compound has structure of formula I:

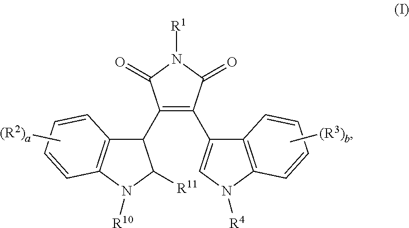

wherein
R¹ is selected from the series consisting of hydrogen, (C₁-C₄)-alkyl, phenyl-(C₁-C₄)-alkyl- and heteroaryl-(C₁-C₄)-alkyl-, wherein phenyl and heteroaryl are unsubstituted or substituted by substituents from the series consisting of halogen, (C₁-C₄)-alkyl and (C₁-C₄)-alkyl-O—;
R² and R³ are independently of each other selected from the series consisting of halogen, (C₁-C₄)-alkyl and (C₁-C₄)-alkyl-O—;
R⁴ is selected from the series consisting of hydrogen and (C₁-C₄)-alkyl;
R¹⁰ is selected from the series consisting of hydrogen, (C₁-C₆)-alkyl and Het¹, wherein (C₁-C₆)-alkyl is unsubstituted or substituted by R²⁰, and wherein Het¹ is unsubstituted or substituted by R²¹;
R¹¹ is selected from the series consisting of hydrogen and (C₁-C₄)-alkyl,
or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl;
R²⁰ is selected from the series consisting of R³⁰—O—, R³¹—N(R³²)—, H₂N—C(═NH)—S—, pyridinyl and Het², wherein Het² is unsubstituted or substituted by R³³;
R²¹ is selected from the series consisting of (C₁-C₄)-alkyl, phenyl-(C₁-C₄)-alkyl- and pyridinyl-(C₁-C₄)-alkyl-, wherein phenyl and pyridinyl are unsubstituted or substituted by substituents from the series consisting of halogen, (C₁-C₄)-alkyl and (C₁-C₄)-alkyl-O—;
R²² is selected from the series consisting of hydrogen and (C₁-C₄)-alkyl;
R²³ is selected from the series consisting of hydrogen, (C₁-C₄)-alkyl, R³¹—N(R³²)— and R³¹—N(R³²)—(C₁-C₄)-alkyl-;
R²⁴ is selected from the series consisting of hydrogen, (C₁-C₄)-alkyl, pyridinyl-(C₁-C₄)-alkyl- and R³¹—N(R³²)—(C₁-C₄)-alkyl-;
R³⁰, R³¹ and R³² are independently of each other selected from the series consisting of hydrogen and (C₁-C₄)-alkyl;
R³³ is selected from the series consisting of (C₁-C₄)-alkyl;
Het¹ is a 4-membered to 7-membered, monocyclic, saturated heterocycle comprising one ring nitrogen atom, which is bonded via a ring carbon atom;
Het² is a 4-membered to 7-membered, monocyclic, saturated heterocycle comprising one or two ring nitrogen atoms, which is bonded via a ring carbon atom or a ring nitrogen atom;
heteroaryl is a 5-membered or 6-membered, monocyclic, aromatic heterocycle comprising one or two identical or different ring heteroatoms selected from the series consisting of N, O and S;
a and b are independently of each other selected from the series consisting of 0, 1 and 2;
m and n are independently of each other selected from the series consisting of 1 and 2.

### Embodiment 2

The method of embodiment 1, wherein in the compound of formula I,
R¹ is selected from the series consisting of hydrogen, (C₁-C₄)-alkyl and phenyl-(C₁-C₄)-alkyl-, wherein phenyl is unsubstituted or substituted by substituents from the series consisting of halogen, (C₁-C₄)-alkyl and (C₁-C₄)-alkyl-O—;
R² and R³ are independently of each other selected from the series consisting of halogen, (C₁-C₄)-alkyl and (C₁-C₄)-alkyl-O—;
R⁴ is selected from the series consisting of hydrogen and (C₁-C₄)-alkyl;
R¹⁰ is selected from the series consisting of (C₁-C₆)-alkyl and Het¹, wherein (C₁-C₆)-alkyl is unsubstituted or substituted by R²⁰, and wherein Het¹ is unsubstituted or substituted by R²¹;
R¹¹ is selected from the series consisting of hydrogen and (C₁-C₄)-alkyl,
or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl;
R²⁰ is selected from the series consisting of R³¹—N(R³²)—, H₂N—C(═NH)—S—, pyridinyl and Het², wherein Het² is unsubstituted or substituted by R³³;
R²¹ is selected from the series consisting of (C₁-C₄)-alkyl, phenyl-(C₁-C₄)-alkyl- and pyridinyl-(C₁-C₄)-alkyl-, wherein phenyl and pyridinyl are unsubstituted or substituted by substituents from the series consisting of halogen, (C₁-C₄)-alkyl and (C₁-C₄)-alkyl-O—;
R²² is selected from the series consisting of hydrogen and (C₁-C₄)-alkyl;
R²³ is selected from the series consisting of hydrogen, (C₁-C₄)-alkyl, R³¹—N(R³²)— and R³¹—N(R³²)—(C₁-C₄)-alkyl-;
R²⁴ is selected from the series consisting of hydrogen, (C₁-C₄)-alkyl, pyridinyl-(C₁-C₄)-alkyl- and R³¹—N(R³²)—(C₁-C₄)-alkyl-;
R³¹ and R³² are independently of each other selected from the series consisting of hydrogen and (C₁-C₄)-alkyl;
R³³ is selected from the series consisting of (C₁-C₄)-alkyl;
Het¹ is a 4-membered to 7-membered, monocyclic, saturated heterocycle comprising one ring nitrogen atom, which is bonded via a ring carbon atom;
Het² is a 4-membered to 7-membered, monocyclic, saturated heterocycle comprising one or two ring nitrogen atoms, which is bonded via a ring carbon atom or a ring nitrogen atom;
a and b are independently of each other selected from the series consisting of 0 and 1;
m and n are independently of each other selected from the series consisting of 1 and 2.

### Embodiment 3

The method of embodiment 1, wherein in the compound of formula I,
R¹ is selected from the series consisting of hydrogen, (C₁-C₄)-alkyl and phenyl-(C₁-C₄)-alkyl-;
R² and R³ are independently of each other selected from the series consisting of halogen, (C₁-C₄)-alkyl and (C₁-C₄)-alkyl-O—;
R⁴ is selected from the series consisting of hydrogen and (C₁-C₄)-alkyl;
R¹⁰ is selected from the series consisting of (C₁-C₆)-alkyl and Het¹, wherein (C₁-C₆)-alkyl is unsubstituted or substituted by R²⁰, and wherein Het¹ is unsubstituted or substituted by R²¹;
R¹¹ is selected from the series consisting of hydrogen and (C₁-C₄)-alkyl,
or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl;
R²⁰ is selected from the series consisting of R³¹—N(R³²)—, H₂N—C(═NH)—S— and Het², wherein Het² is unsubstituted or substituted by R³³;
R²¹ is selected from the series consisting of (C₁-C₄)-alkyl and pyridinyl-(C₁-C₄)-alkyl-, wherein pyridinyl is unsubstituted or substituted by substituents from the series consisting of halogen, (C₁-C₄)-alkyl and (C₁-C₄)-alkyl-O—;
R²² is hydrogen;
R²³ is selected from the series consisting of hydrogen, (C₁-C₄)-alkyl, R³¹—N(R³²)— and R³¹—N(R³²)—(C₁-C₄)-alkyl-;
R²⁴ is selected from the series consisting of hydrogen, (C₁-C₄)-alkyl and pyridinyl-(C₁-C₄)-alkyl-;
R³¹ and R³² are independently of each other selected from the series consisting of hydrogen and (C₁-C₄)-alkyl;
R³³ is selected from the series consisting of (C₁-C₄)-alkyl;
Het¹ is a 5-membered or 6-membered, monocyclic, saturated heterocycle comprising one ring nitrogen atom, which is bonded via a ring carbon atom;
Het² is a 4-membered to 6-membered, monocyclic, saturated heterocycle comprising one or two ring nitrogen atoms, which is bonded via a ring carbon atom or a ring nitrogen atom;
a and b are independently of each other selected from the series consisting of 0 and 1;
m and n are independently of each other selected from the series consisting of 1 and 2.

### Embodiment 4

The method of embodiment 1, wherein in the compound of formula I, R¹ is hydrogen;
R⁴ is selected from the series consisting of hydrogen and (C₁-C₄)-alkyl;
R¹⁰ is selected from the series consisting of (C₁-C₄)-alkyl and Het¹, wherein (C₁-C₄)-alkyl is unsubstituted or substituted by R²⁰, and wherein Het¹ is unsubstituted or substituted by R²¹;
R¹¹ is hydrogen,
or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—;
R²⁰ is selected from the series consisting of R³¹—N(R³²)—, H₂N—C(═NH)—S— and Het², wherein Het² is unsubstituted or substituted by R³³;
R²¹ is selected from the series consisting of pyridinyl-(C₁-C₄)-alkyl-;
R²² is hydrogen;
R²³ is selected from the series consisting of R³¹—N(R³²)— and R³¹—N(R³²)—(C₁-C₄)-alkyl-;
R²⁴ is selected from the series consisting of hydrogen and (C₁-C₄)-alkyl;
R³¹ and R³² are independently of each other selected from the series consisting of hydrogen and (C₁-C₄)-alkyl;
R³³ is selected from the series consisting of (C₁-C₄)-alkyl;
Het¹ is a 5-membered or 6-membered, monocyclic, saturated heterocycle comprising one ring nitrogen atom, which is bonded via a ring carbon atom;
Het² is a 4-membered to 6-membered, monocyclic, saturated heterocycle comprising one or two ring nitrogen atoms, which is bonded via a ring carbon atom or a ring nitrogen atom;
a and b are 0;
m is 2 and n is 1.

### Embodiment 5

The method of embodiment 1, wherein in the compound of formula I,
R¹ is hydrogen;
R⁴ is selected from the series consisting of hydrogen and (C₁-C₄)-alkyl;
R¹⁰ is selected from the series consisting of (C₁-C₄)-alkyl, wherein (C₁-C₄)-alkyl is unsubstituted or substituted by R²⁰;
R¹¹ is hydrogen,
or R¹⁰ and R¹¹ together are a divalent group selected from the series consisting of the groups —(CH₂)ₘ—C(R²²)(R²³)—(CH₂)ₙ—;
R²⁰ is selected from the series consisting of R³¹—N(R³²)—, H₂N—C(═NH)—S— and Het², wherein Het² is unsubstituted or substituted by R³³;
R²² is hydrogen;
R²³ is selected from the series consisting of R³¹—N(R³²)— and R³¹—N(R³²)—(C₁-C₄)-alkyl-;
R³¹ and R³² are independently of each other selected from the series consisting of hydrogen and (C₁-C₄)-alkyl;
R³³ is selected from the series consisting of (C₁-C₄)-alkyl;
Het² is a 5-membered to 6-membered, monocyclic, saturated heterocycle comprising one or two ring nitrogen atoms, which is bonded via a ring carbon atom or a ring nitrogen atom;
a and b are 0;
m is 2 and n is 1.

### Embodiment 6

The method of embodiment 1, wherein in the compound of formula I,
R¹ is hydrogen;
R⁴ is selected from the series consisting of hydrogen and (C₁-C₄)-alkyl;
R¹⁰ is selected from the series consisting of (C₁-C₄)-alkyl, wherein (C₁-C₄)-alkyl is unsubstituted or substituted by R²⁰;
R¹¹ is hydrogen,
or R¹⁰ and R¹¹ together are a divalent group selected from the series consisting of the groups —(CH₂)ₘ—C(R²²)(R²³)—(CH₂)ₙ—;
R²⁰ is selected from the series consisting of R³¹—N(R³²)— and Het², wherein Het² is unsubstituted or substituted by R³³;
R²² is hydrogen;
R²³ is selected from the series consisting of R³¹—N(R³²)—(C₁-C₄)-alkyl-;
R³¹ and R³² are independently of each other selected from the series consisting of hydrogen and (C₁-C₄)-alkyl;
R³³ is selected from the series consisting of (C₁-C₄)-alkyl;
Het² is a 5-membered to 6-membered, monocyclic, saturated heterocycle comprising one ring nitrogen atom, which is bonded via a ring carbon atom;
a and b are 0;
m is 2 and n is 1.

### Embodiment 7

The method of embodiment 1, wherein in the compound of formula I,
R¹ is hydrogen;
R⁴ is selected from the series consisting of (C₁-C₄)-alkyl;
R¹⁰ and R¹¹ together are a divalent group selected from the series consisting of the groups —(CH₂)ₘ—C(R²²)(R²³)—(CH₂)ₙ—;
R²² is hydrogen;
R²³ is selected from the series consisting of R³¹—N(R³²)—(C₁-C₄)-alkyl-;
R³¹ and R³² are independently of each other selected from the series consisting of hydrogen and (C₁-C₄)-alkyl;
a and b are 0;
m is 2 and n is 1.

### Embodiment 8

The method of embodiment 1, wherein in the compound of formula I, the compound chemical name is 3-(8-aminomethyl-6,7,8,9-tetrahydro-pyrido[1,2-a]indol-10-yl)-4-(1-methyl-1H-indol-3-yl)-pyrrole-2,5-dione; and has structure of formula I-1:

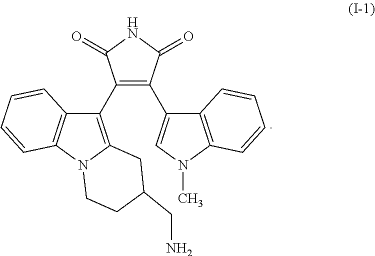 [SMILES: Cn1cc(C2=C(c3c4n(c5ccccc35)CCC(CN)C4)C(=O)NC2=O)c2ccccc21]

### Embodiment 9

The method of claim 1, wherein the cells are mesenchymal stem cells (MSCs); stem/progenitor cells including skeletal muscle-derived stem/progenitor cells (MDSPCs), satellite cells, hematopoietic stem/progenitor cells, bone or bone marrow derived stem/progenitor cells, neural stem/progenitor cells, eye stem/progenitor cells, liver derived stem-progenitor cells, brain derived stem/progenitor cells, heart/cardiac derived stem/progenitor cells, intestinal stem/progenitor cells, mesenchymal stem/progenitor cells, skin stem/progenitor cells, hair/hair follicle stem/progenitor cells, endothelial stem/progenitor cells, epithelial stem/progenitor cells, olfactory adult stem/progenitor cells, neural crest stem/progenitor cells, testicular stem/progenitor cells, embryonic stem cells, placental derived stem/progenitor cells, amniotic fluid-derived stem/progenitor cells, mucosal stem/progenitor cells, cord blood stem/progenitor cells, LGRS+ stem or progenitor cells, and inducible pluripotent stem cells; progeny cells of the foregoing; or modified or engineered cells of the foregoing. In an embodiment, a cell is of a type described in any of EP1491093B1, U.S. Pat. No. 6,787,355, US20130336935, CA2816489A1, U.S. Pat. No. 7,947,266 B2, and WO2002078449A2. In embodiments, the cells can further include any of: cells derived from islets including but not limited to beta cells, delta cells, alpha cells, acinar cells; programmed and reprogrammed cells and their progeny; mesenchymal stem cells derived from cells that have been reprogrammed to progenitors or stem cells or programmed directly to MSCs; osteochondroreticular stem/progenitor cells; connective tissue progenitor cells; and multipotent adult progenitor cells.

### Embodiment 10

The method of any one of the foregoing embodiments, wherein the disease or injury condition is an inflammatory condition.

### Embodiment 11

The method of any one of the foregoing claims, wherein the cells are MSCs.

### Embodiment 12

The method of any one of the foregoing embodiments, wherein the cells are mammalian MSCs.

### Embodiment 13

The method of any one of the foregoing embodiments, wherein the cells are human MSCs.

### Embodiment 14

The method of any one of the foregoing embodiments, wherein the compound is capable of increasing a cell surface expression level of CD11a by MSCs.

### Embodiment 15

The method of any one of the foregoing embodiments, wherein the effective amount of the compound composition is a concentration from about 0.01 micromolar to about 10 micromolar, wherein optionally the concentration is from about 0.1 micromolar to about 3 micromolar. In an embodiment, the concentration is from 0.01 to 100 micromolar. In an embodiment, the concentration is from 0.5 to 30 micromolar. In an embodiment, the concentration is 0.01 to 10 micromolar. In an embodiment, the concentration is 0.1 to 3 micromolar.

### Embodiment 16

A method of improving a homing function of mesenchymal stem cells (MSCs), the method comprising the steps of:
(a) providing MSCs; and
(b) contacting the MSCs with a composition comprising a compound as described herein, wherein the compound is capable of improving a homing function of MSCs;
wherein the homing function is one or more of
   (i) increased expression of a cell surface molecule capable of facilitating a homing function, wherein optionally the cell surface molecule is CD11a,
   (ii) increased in vitro adhesion by the MSCs in a shear flow assay,
   (iii) increased binding of E-selectin or ICAM-1, and
   (iv) increased homing and/or anti-inflammatory activity of the MSCs upon in vivo systemic administration of the MSCs in an animal inflammation model.

### Embodiment 17

The method of embodiment 16, wherein the compound is of formula I or any of formulas I-1 to I-19.

### Embodiment 18

A composition comprising purified treated MSCs, wherein the purified treated MSCs are produced by contacting the MSCs with a compound as described herein, such as of formula I or any of formulas I-1 to 1-19.

### Embodiment 19

A pharmaceutical composition comprising an effective amount of purified treated MSCs, wherein the purified treated MSCs are produced by contacting the MSCs with a compound as described herein, such as of formula I or any of formulas I-1 to 1-19, and a pharmaceutical carrier.

### Embodiment 20

The pharmaceutical composition of embodiment 19, wherein the purified treated MSCs comprise pharmaceutical agents comprising therapeutic molecules, wherein the therapeutic molecules optionally comprise proteins.

### Embodiment 21

A composition comprising a combination of purified MSCs in vitro and an effective amount of a compound as described herein, wherein the compound is capable of improving a homing function of MSCs, wherein the compound is optionally of formula I or any of formulas I-1 to 1-19.

### Embodiment 22

A method of screening to identify a small molecule compound capable of improving a homing function of MSCs, comprising the steps of:
(a) providing a candidate composition comprising a candidate small molecule compound;
(b) providing MSCs;
(c) treating the MSCs with the candidate composition, thereby generating treated MSCs;
(d) measuring a characteristic of the treated MSCs, wherein the characteristic comprises one or more of in vitro expression of a cell surface molecule capable of facilitating a homing function, in vitro adhesion of the treated MSCs in a shear flow assay, and anti-inflammatory activity upon in vivo systemic administration in an animal inflammation model;
(e) comparing one or more of the characteristics of treated MSCs relative to a characteristic of negative control MSCs, wherein the negative control MSCs are untreated or treated with a negative control candidate composition which is not capable of improving a homing function of MSCs; and
(f) identifying the small molecule compound capable of improving a homing function of MSCs wherein the small molecule compound, for treated MSCs relative to negative control MSCs, demonstrates one or more of increased expression of a cell surface molecule capable of facilitating a homing function, increased in vitro adhesion in a shear flow assay, reduced autoimmune disease activity upon in vivo systemic administration in an animal model, and increased anti-inflammatory activity upon in vivo systemic administration in an animal inflammation model.

### Embodiment 23

The method of embodiment 22, wherein the cell surface molecule comprises CD11a, wherein the shear flow assay uses an E-selectin coated substrate, and the animal inflammation model is a mouse inflamed ear model.

### Embodiment 24

The method of embodiment 22, wherein the shear flow assay of step (d) is an in vitro firm adhesion assay comprising the steps of:
(d-a) providing an assay plate comprising multiple wells wherein a microfluidic channel connects each pair of adjacent inlet and outlet wells;
(d-b) placing the assay plate under vacuum;
(d-c) coating the channels with recombinant human E-selectin or ICAM-1 and incubating for a time to allow sufficient coating;
(d-d) washing the wells;
(d-e) introducing compound-pretreated MSCs into the channel and allowing a time period for attachment without a flow being applied;
(d-f) subjecting putatively attached cells to increasing shear flow, optionally ranging from about 0.25 dynes/cm2 to about up to 10 dynes/cm2;
(d-g) obtaining data from observation of firmly adhered cells, optionally from acquired image data.

### Embodiment 25

A method of treating a subject with a disease or injury condition, the method comprising the steps of:
(a) providing mesenchymal stem cells (MSCs);
(b) contacting the MSCs with an effective amount of a compound composition, wherein the compound is capable of improving a homing function of MSCs, thereby generating a composition comprising pretreated MSCs; and
(c) administering an effective amount of the composition comprising pretreated MSCs to the subject;
wherein the compound is a selective protein kinase C (PKC) inhibitor.

### Embodiment 26

A pharmaceutical composition comprising an effective amount of a compound as described herein, wherein the compound is capable of improving a homing function of MSCs, and a pharmaceutical carrier, wherein the compound optionally is of formula I or any of formulas I-1 to I-19.

### Embodiment 27

A purified in vitro culture of MSCs, wherein the MSCs express an increased cell surface level of CD11a relative to a level corresponding to that of cultured MSCs, wherein the increased cell surface level is of endogenous and non-engineered origin. In an embodiment, the purified in vitro culture of MSCs are pretreated with a compound as described herein, and the cultured MSCs are untreated as a negative control for comparison.

In an embodiment, an increased surface expression level that is of endogenous and non-engineered origin is achieved by a cell's existing internal pathway of gene and protein expression (which may be modified by pretreatment with a compound described herein), in contrast to expression resulting from genetic engineering involving an exogenously introduced gene or vector construct of recombinant DNA technology. In an embodiment, provided is a composition comprising purified MSCs in vitro, wherein the MSCs are pretreated with a compound of the invention as described herein, and wherein the MSCs express an increased level relative to untreated MSCs of cell surface expression of CD11a, ICAM-1 binding activity, and/or E-selectin binding activity.

### Embodiment 28

The method of embodiment 1, further comprising the steps before step (c) of:
(b″) freezing the pretreated MSCs, thereby generating frozen pretreated MSCs, and
(b′″) thawing the frozen pretreated MSCs.

### Embodiment 29

A chilled or frozen composition of purified pretreated MSCs, wherein the purified pretreated MSCs have been previously subject to pretreatment with an effective amount of the compound having formula I and are capable of an enhanced homing function relative to untreated MSCs, and wherein the chilled or frozen composition has a temperature of equal to or lower than 4° C., equal to or lower than −20° C., or equal to or lower than −80° C.; wherein the chilled or frozen composition optionally comprises a cryoprotectant.

### Embodiment 30

A method of manufacturing a therapeutic composition of pretreated MSCs capable of an enhanced homing function, comprising the steps of:
(a) providing a population of MSCs;
(b) pretreating the MSCs by contacting the MSCs with a composition comprising a compound as described herein, wherein the compound is capable of improving a homing function of MSCs; wherein the compound optionally is of formula I or any of formulas I-1 to 1-19, and wherein the homing function is one or more of
   (i) increased expression of a cell surface molecule capable of facilitating a homing function, wherein optionally the cell surface molecule is CD11a,
   (ii) increased in vitro adhesion by the MSCs in a shear flow assay,
   (iii) increased binding of E-selectin or ICAM-1, and
   (iv) increased homing and/or anti-inflammatory activity of the MSCs upon in vivo systemic administration of the MSCs in an animal inflammation model,
(c) optionally preparing a single dose aliquot or multi-dose aliquot of the composition of pretreated MSCs; and
(d) optionally freezing the pretreated MSCs, thereby generating frozen pretreated MSCs; thereby generating the therapeutic composition of pretreated MSCs.

### Embodiment 31

A composition comprising purified MSCs in vitro, wherein the MSCs are pretreated with a compound described herein, and wherein the MSCs express an increased level of one or more of: (a) cell surface expression of CD11a, (b) binding activity to ICAM-1, and (c) binding activity to E-selectin; wherein the increased level is relative to a corresponding level for control MSCs, wherein the control MSCs are optionally untreated or stimulated with a negative control compound. In an embodiment, a negative control compound is ruboxistaurin or DMSO.

In an embodiment, contacting of cells for compound pretreatment occurs in vitro.

In an embodiment, the method includes providing the MSCs with a cryoprotectant for cryopreservation in connection with the freezing step. In an embodiment, the method includes providing a therapeutically effective amount of the composition of pretreated MSCs in connection with the aliquot preparing step. In an embodiment, the method includes providing the MSCs with a pharmaceutically acceptable carrier.

In embodiments of the invention, cells are delivered to any tissue or organ. In embodiments, the organ is any of bone marrow, blood, spleen, liver, lung, intestinal tract, brain, immune system, circulatory system, bone, connective tissue, muscle, heart, blood vessels, pancreas, central nervous system, peripheral nervous system, kidney, bladder, skin, eye, epithelial appendages, breast-mammary glands, fat tissue, and mucosal surfaces including oral esophageal, vaginal and anal. In embodiments, diseases suitable for employment of compositions and methods are cancer, cardiovascular disease, metabolic disease, liver disease, diabetes, hepatitis, hemophilia, degenerative or traumatic neurological conditions, autoimmune disease, genetic deficiency, connective tissue disorders, anemia, infectious disease and transplant rejection. In embodiments, pretreated cells are used for healing or regeneration of tissues or organs.

### BRIEF DESCRIPTION OF THE DRAWINGS

FIG. 1 illustrates in (1A) an overview of a multi-step screening process for identification of small molecules which can affect homing of MSCs by measuring cell surface expression of molecules, adhesion of cells, and the potential anti-inflammatory impact upon systemic administration of cell therapy. (1B) shows the results of surface expression of CD11a. (1C) indicates the structure of a generic formula (I) for compounds in embodiments relating to the invention and of a particular compound Ro 31-8425, also designated 1929 with reference to the hydrochloride salt form of the compound. (1D) shows a graph of surface expression of CD11a as a function of the concentration level of treatment of cells with compound 1929. The results of measuring CD11a expression levels by flow cytometry fluorescence technique are shown in (1E) with high expression levels on promyelocytic leukemia cells (HL-60, positive control) and (1F) a lack of CD11a on the surface of culture expanded MSCs.

FIG. 2 illustrates the results of pretreatment of MSCs with control and test compounds. (2A) Microscopy of MSCs firm adhesion to an E-selectin-coated surface following pretreatment with 1927 and 1929 (10× magnification). (2B) Quantification of MSC firm adhesion to an E-selectin surface as a function of pretreatment regimen. (2C) Antibody blocking experiments demonstrate a direct involvement of CD11a in the increased firm adhesion of 1929-treated MSCs to E-selectin surface.

FIG. 3 illustrates that pretreated MSCs exhibit increased homing to inflamed sites and an improved anti-inflammatory impact following systemic administration. (3A) Homing of systemically infused MSCs to LPS-induced inflamed mouse ears was assessed 24 hr following cell infusion. Example images (scale bar=50 μm) demonstrate homing to the inflamed ear of small molecule pre-treated MSCs (blue cells in upper panels, depicted in lower panels with solid border) compared to vehicle-treated MSCs (green cells in upper panels; depicted in lower panels with broken border and “G”). Left and right panels show results from pretreatment with compounds 1927 and 1929 respectively. (3B) shows the percent increase in MSC homing to an inflamed ear after compound pretreatment. When quantified, pretreatment with compound 1927 did not affect MSC homing to the inflamed ear versus the vehicle-treated control (n=4 mice). Pretreatment with compound 1929 significantly promoted MSC homing versus the vehicle-treated control and 1927-treated cells (n=8 mice). (3C) 1929-treated MSCs displayed a superior effect in reducing swollen ear thickness of the inflamed ear compared to native MSCs (n=8 mice). (3D) MSCs treated with 1929 significantly reduced TNF-α level in the inflamed ear compared to the control ear.

FIG. 4 illustrates results of medium-throughput screening for detection of cell surface expression of CD11a. The results demonstrate the robustness of the screening. The fluorescence signal averages were 1273±392 relative fluorescence units (RFU) for vehicle-treated MSC cells and 122604±27863 RFU for HL-60 cells. Results of signal to background (S/B) ratio (left y-axis for thin columns) and Z′ value (right y-axis for top line with square symbols) are indicated as a function of multiwell plates grouped by screening runs.

FIG. 5 illustrates in the results of surface expression of CD11a measured by fluorescence flow cytometry as a function of concentration of compound 1929 for pretreatment of MSCs.

FIG. 6 illustrates results of cell viability of MSCs upon contacting with certain small molecule compounds as a function of compound concentration. The quantification of MSC viability in response to 24 hours pretreatment with 1927 or 1929 was observed; these compounds did not modify MSC viability. Error bars represent standard deviation.

FIG. 7 illustrates the results of analysis of certain secretome components including the amount of analytes secreted by MSC, including IL-6, IL-8, SDF-la, MCP-1, and VEGF, upon priming (treatment) of MSC with compound concentration of 3 micromolar for compound 1927 (FIG. 7A) and compound 1929 (FIG. 7B). Error bars represent standard deviation.

FIG. 8 illustrates in FIG. 8A the percentage of CD11a+ cells of an MSC population as a function of pretreatment compound concentration. FIG. 8B illustrates mass cytometry data with detection of CD11a levels on MSCs pretreated with compound.

FIG. 9 illustrates in FIG. 9A mass cytometry data for the level of CD11a surface expression on MSCs over time. FIG. 9B shows a graph of the fold increase in CD11a mRNA levels as a function of time post pretreatment for MSCs treated with compound versus vehicle control.

FIG. 10 illustrates in FIG. 10A the percentage of CD11a positive cells from the MSC population as a function of MSC donor source for MSCs treated with compound versus vehicle control. The results indicated no donor-dependent effect, thus the pretreatment can robustly induce CD11a surface expression independently of the donor source. FIG. 10B illustrates that pretreatment with ruboxistaurin as a test compound does not upregulate CD11a surface expression on MSCs. MSCs were pretreated with DMSO vehicle control (DMSO-MSC, 0.1%) or ruboxistaurin (Rub-MSC, 3 μM) for 24 h and CD11a expression levels were assessed via flow cytometry (cells were stained with either FITC-CD11a Ab or isotype control, representative data from n=3 independent experiments).

FIG. 11 illustrates in FIG. 11A the results of microscope images from firm adhesion assays to detect binding of pretreated MSCs to an ICAM-1 coated surface under dynamic conditions of shear flow. Tested compounds included Ro-31-8425, ruboxistaurin, and vehicle control. FIG. 11B shows graphic results of firm adhesion assays with the percent of adhered cells after flow versus the type of pretreatment condition for MSCs. FIG. 11C provides a pie chart of the proportions of the pretreated MSC cell population in the categories of (i) native expression of ICAM-I binding domains, (ii) lacking expression of ICAM-1 binding domains, (iii) compound-induced expression of CD11a, and (iv) compound-induced expression/activation of other ICAM-1 binding domains.

FIG. 12 illustrates in FIG. 12A the results of firm adhesion assays which were conducted in the context of antibody blocking experiments for MSCs pretreated with compound RO-31-8425 or vehicle control. The assays employed a surface coated with ICAM-1. FIG. 12B shows the structure of the compound ruboxistaurin.

FIG. 13 illustrates in FIG. 13A a microscopic image of the results of in vivo testing of pretreated MSCs for homing activity. Relative to vehicle control, MSCs pretreated with compound RO-31-8425 demonstrated enhanced homing activity. (white bar, 50 microns) FIG. 13B shows graphic results of the number of cells found in an inflamed ear and the control ear for MSCs as a function of pretreatment condition using compound RO-31-8425 versus vehicle control.

FIG. 14 illustrates the result of in vivo homing of pretreated MSCs further in the context of antibody-blocking conditions.

FIG. 15 illustrates in FIG. 15A the results of studying the effect of pretreatment with compound Ro-31-8425 on cell viability of MSCs. FIG. 15B shows the results of measuring the effect of Ro-31-8425 on levels of CD18 mRNA expression by MSCs.

### DETAILED DESCRIPTION

In general the terms and phrases used herein have their art-recognized meaning, which can be found by reference to standard texts, journal references and contexts known to those skilled in the art. The following definitions are provided to clarify their specific use in the context of the disclosure.

As used herein, the term “mesenchymal stem cells” (MSCs) refers to a type of stem cells which are multipotent but not totipotent, where the cells are capable of self-renewal and can give rise to a number of unique, differentiated mesenchymal cell types. In embodiments, MSCs are capable of differentiation into a variety of cell lineages including osteoblasts, adipocytes, and chondroblasts under certain standard in vitro differentiating conditions. In embodiments of human mesenchymal stem cells (huMSCs), the term relates to cells characterized by attributes of adhering to tissue culture plastic, being positive for certain markers including CD105, CD73, and CD90, and being negative for CD45, CD34, CD14 or CD11b, CD79a, or CD19 and HLA-DR. A person of ordinary skill in the art will recognize further that in embodiments, a manipulated MSC may not always maintain an exact phenotype of surface molecule expression; for example, upon activation with interferon, MSC may express HLA-DR which does not signify that the cell is no longer an MSC. As another example, a cultured MSC may not express a particular marker or may change its pattern of marker expression yet may not cease to be considered an MSC. In embodiments, cells in the art referred to as pericytes may be considered MSCs. In embodiments, MSCs are derived from sources including bone marrow, umbilical cord blood, adipose tissue, placenta, Wharton's jelly, and other tissues or organs. In embodiments, the MSCs are mammalian MSCs. In particular embodiments, the MSCs are human MSCs.

As used herein, the term “homing” refers to the delivery or migration in the body of a cell or cells to the site of pathology or a desired target tissue, such as in the context of an injury or disease. In embodiments the term relates to targeted migration to the sites of ischemic, inflammatory or mechanical injury or site of tumor growth and recruitment within and around the damaged or abnormal area. Further in the context of cell therapy, the term can refer to activity relating to cellular circulation throughout the body via the circulatory system until the cell is arrested by or near microvascular endothelial cells at a target tissue or organ, following a coordinated multistep process including adhesion to the endothelium, transendothelial migration, chemotaxis, matrix degradation and invasion. In an embodiment, the term may also include the active recruitment of neighboring or distant endogenous cells, including stem/progenitor cells, to a desired anatomic compartment for therapeutic applications.

As used herein, the term “homing function” refers to a characteristic of a mesenchymal stem cell, relative to that of an untreated or negative control cell or value for such characteristic, including one or more of expressing a cell surface molecule capable of binding to an endothelial surface molecule associated with upregulation at an inflammation site, adhering in an in vitro adhesion assay employing shear flow, and migrating to an inflammatory site upon in vivo systemic administration in a subject with inflammation or an animal inflammation model.

As used herein, the terms “pretreatment” and “pretreated” versus “treated” may be understood in the particular contexts as set forth. For example, the terms may be equivalent in the sense of embodiments referring to contacting MSCs with a compound composition such that the contacted MSCs are induced to an increased state of homing efficiency. In certain circumstances, the prefix of “pre-” and term pretreatment may simply serve as a convenient reminder that the cells are first contacted ex vivo with an inducing compound. Then those “pretreated” cells are used in a medical treatment of exogenous cell therapy for patients in a clinical setting, e.g., a patient who is in need of treatment is treated by administering compositions of homing-improved cells to the patient.

As used herein, the term “purified” can be understood in embodiments to refer to a state of enrichment or selective enrichment of a particular component relative to an earlier state of crudeness or constituency of another component. In embodiments, the term can be considered to correspond to a material that is at least partially purified as opposed to a state of absolute purity. For example in a particular embodiment, a composition or population of MSCs can be considered purified even if the composition or population does not reach a level of one hundred percent purity either with respect to other components in the composition or other cell types in the population.

If structural elements such as groups or substituents, for example, can occur several times in the compounds of the formula I, they are all independent of each other and can in each case have any of the specified meanings, and they can in each case be identical to or different from any other such element. For example, two or more substituents R²⁰ in an alkyl group representing R¹⁰ can be identical or different, and in a dialkylamino group, for example in such a group representing R³¹—N(R³²)—, the alkyl groups can be identical or different.

Alkyl groups, i.e. saturated hydrocarbon residues, can be linear, i.e. straight-chained, or branched. This also applies if these groups are substituted or are part of another group, for example an alkyl-O— group (alkyloxy group, alkoxy group). Depending on the respective definition, the number of carbon atoms in an alkyl group can be 1, 2, 3, 4, 5 or 6, or 1, 2, 3 or 4, or 1, 2 or 3, or 1 or 2, or 1, for example. Examples of alkyl are methyl, ethyl, propyl including n-propyl and isopropyl, butyl including n-butyl, sec-butyl, isobutyl and tert-butyl, pentyl including n-pentyl, 1-methylbutyl, isopentyl, neopentyl and tert-pentyl, and hexyl including n-hexyl, 3,3-dimethylbutyl and isohexyl. Examples of alkyl-O— groups are methoxy, ethoxy, n-propoxy, isopropoxy, n-butoxy, isobutoxy, tert-butoxy. A substituted alkyl group can be substituted in any positions, provided that the respective compound is sufficiently stable and is suitable as a pharmaceutical active compound. In general, the term “substituted” refers to the replacement of one or more hydrogen atoms in a given structure with the residue or residues of the substituents which are specified in the definition of the respective group in the compounds of the formula I.

The above explanations with respect to alkyl groups apply correspondingly to alkyl groups which in the definition of a group in the compounds of the formula I are bonded to two adjacent groups, or linked to two groups, and may be regarded as divalent alkyl groups (alkanediyl groups, alkylene groups), like in the case of the alkyl part of a substituted alkyl group. Thus, such groups can also be linear or branched, and the bonds to the adjacent groups can be located in any positions and can start from the same carbon atom or from different carbon atoms.

In substituted phenyl groups the substituents can be located in any positions. In monosubstituted phenyl groups, the substituent can be located in position 2, in position 3 or in position 4. In disubstituted phenyl groups, the substituents can be located in positions 2 and 3, in positions 2 and 4, in positions 2 and 5, in positions 2 and 6, in positions 3 and 4, or in positions 3 and 5. In trisubstituted phenyl groups, the substituents can be located in positions 2, 3 and 4, in positions 2, 3 and 5, in positions 2, 3 and 6, in positions 2, 4 and 5, in positions 2, 4 and 6, or in positions 3, 4 and 5.

Examples of pyridinyl groups are pyridin-2-yl, pyridin-3-yl and pyridin-4-yl, which can all be unsubstituted or substituted as specified in the definition of the compounds of the formula I.

Examples of aromatic heterocyclic ring systems, from which the group heteroaryl in the compounds of the formula I can be derived, and from any one or more of which the group heteroaryl is selected in one embodiment of the invention, are furan, thiophene, pyrrole, isoxazole ([1,2]oxazole), oxazole ([1,3]oxazole), isothiazole ([1,2]thiazole), thiazole ([1,3]thiazole), pyrazole, imidazole, pyridine, pyridazine, pyrimidine and pyrazine, which can all be unsubstituted or substituted in any suitable positions as specified in the definition of the compounds of the formula I. In one embodiment of the invention, the substituents on a ring nitrogen atom in a 5-membered heterocycle which carries a hydrogen atom, such as in the case of pyrrole, pyrazole and imidazole, is selected from the series consisting of (C₁-C₄)-alkyl. Heteroaryl groups can be bonded via any suitable ring atom. In one embodiment of the invention a heteroaryl group is bonded via any ring carbon atom. For example, a furan ring, a thiophene ring and a pyrrole ring can be bonded via positions 2 and 3, an isoxazole ring, an isothiazole ring and a pyrazole ring via positions 3, 4 and 5, an oxazole ring, a thiazole ring and an imidazole via positions 2, 4 and 5, a pyridine ring via positions 2, 3 and 4, a pyrimidine ring via positions 2, 4 and 5, a pyrazine ring via position 2.

Halogen is fluorine, chlorine, bromine or iodine. In one embodiment of the invention, in any of its occurrences halogen is fluorine, chlorine or bromine, in another embodiment fluorine or chlorine, in another embodiment fluorine, in another embodiment chlorine, wherein all occurrences of halogen are independent of each other.

Embodiments of the present invention include all stereoisomeric forms of the compounds of the formula I, for example all enantiomers and diastereomers including cis/trans isomers. Embodiments of the invention likewise include mixtures of two or more stereoisomeric forms, for example mixtures of enantiomers and/or diastereomers including cis/trans isomers, in all ratios. Asymmetric centers in the compounds of the formula I can all independently of each other have S configuration or R configuration. Embodiments of the invention relate to enantiomers, both the levorotatory and the dextrorotatory antipode, in enantiomerically pure form and essentially enantiomerically pure form, for example with a molar ratio of the two enantiomers of about 98:2 or greater, or about 99:1 or greater, and in the form of their racemate, i.e. a mixture of the two enantiomers in molar ratio of 1:1, and in the form of mixtures of the two enantiomers in all ratios. Embodiments of the invention likewise relate to diastereomers in the form of pure and essentially pure diastereomers and in the form of mixtures of two or more diastereomers in all ratios.

Besides the free compounds of the formula I, i.e. compounds in which acidic and basic groups are not present in the form of a salt, embodiments of the present invention comprise also salts of the compounds of the formula I, in particular their physiologically acceptable salts, or toxicologically acceptable salts, or pharmaceutically acceptable salts, which can be formed on one or more acidic or basic groups in the compounds of the formula I. The compounds of the formula I may thus be deprotonated on an acidic group and be used as alkali metal salts, for example. Compounds of the formula I comprising at least one basic group may also be prepared and used in the form of their acid addition salts, for example in the form of physiologically acceptable salts with inorganic acids and organic acids, such as salts with hydrochloric acid and thus be present in the form of the hydrochlorides, for example.

The term “compound” is defined herein to include pharmaceutically acceptable salts, solvates, hydrates, polymorphs, enantiomers, diastereoisomers, racemates and the like of the compounds having formulae as set forth herein.

### Compounds and Compositions

### Formula I

In embodiments of the invention, compositions are provided comprising one or more compounds. In an embodiment, a compound has formula I:

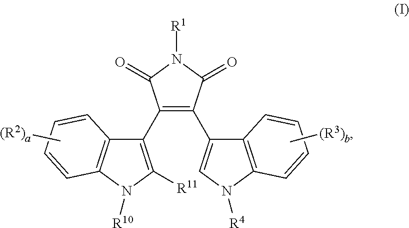

wherein

R¹ is selected from the series consisting of hydrogen, (C₁-C₄)-alkyl, phenyl-(C₁-C₄)-alkyl- and heteroaryl-(C₁-C₄)-alkyl-, wherein phenyl and heteroaryl are unsubstituted or substituted by substituents from the series consisting of halogen, (C₁-C₄)-alkyl and (C₁-C₄)-alkyl-O—;

R² and R³ are independently of each other selected from the series consisting of halogen, (C₁-C₄)-alkyl and (C₁-C₄)-alkyl-O—;

R⁴ is selected from the series consisting of hydrogen and (C₁-C₄)-alkyl;

R¹⁰ is selected from the series consisting of hydrogen, (C₁-C₆)-alkyl and Het¹, wherein (C₁-C₆)-alkyl is unsubstituted or substituted by R²⁰, and wherein Het¹ is unsubstituted or substituted by R²¹;

R¹¹ is selected from the series consisting of hydrogen and (C₁-C₄)-alkyl,

or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl;

R²⁰ is selected from the series consisting of R³⁰—O—, R³¹—N(R³²)—, H₂N—C(═NH)—S—, pyridinyl and Het², wherein Het² is unsubstituted or substituted by R³³;

R²¹ is selected from the series consisting of (C₁-C₄)-alkyl, phenyl-(C₁-C₄)-alkyl- and pyridinyl-(C₁-C₄)-alkyl-, wherein phenyl and pyridinyl are unsubstituted or substituted by substituents from the series consisting of halogen, (C₁-C₄)-alkyl and (C₁-C₄)-alkyl-O—;

R²² is selected from the series consisting of hydrogen and (C₁-C₄)-alkyl;

R²³ is selected from the series consisting of hydrogen, (C₁-C₄)-alkyl, R³¹—N(R³²)— and R³¹—N(R³²)—(C₁-C₄)-alkyl-;

R²⁴ is selected from the series consisting of hydrogen, (C₁-C₄)-alkyl, pyridinyl-(C₁-C₄)-alkyl- and R³¹—N(R³²)—(C₁-C₄)-alkyl-;

R³⁰, R³¹ and R³² are independently of each other selected from the series consisting of hydrogen and (C₁-C₄)-alkyl;

R³³ is selected from the series consisting of (C₁-C₄)-alkyl;

Het¹ is a 4-membered to 7-membered, monocyclic, saturated heterocycle comprising one ring nitrogen atom, which is bonded via a ring carbon atom;

Het² is a 4-membered to 7-membered, monocyclic, saturated heterocycle comprising one or two ring nitrogen atoms, which is bonded via a ring carbon atom or a ring nitrogen atom;

heteroaryl is a 5-membered or 6-membered, monocyclic, aromatic heterocycle comprising one or two identical or different ring heteroatoms selected from the series consisting of N, O and S;

a and b are independently of each other selected from the series consisting of 0, 1 and 2;

m and n are independently of each other selected from the series consisting of 1 and 2.

In one embodiment of the invention, R¹ is selected from the series consisting of hydrogen, (C₁-C₄)-alkyl and phenyl-(C₁-C₄)-alkyl-, wherein phenyl is unsubstituted or substituted by substituents from the series consisting of halogen, (C₁-C₄)-alkyl and (C₁-C₄)-alkyl-O—, in another embodiment from the series consisting of hydrogen, (C₁-C₄)-alkyl and phenyl-(C₁-C₄)-alkyl-, wherein phenyl is unsubstituted, in another embodiment from the series consisting of hydrogen and (C₁-C₄)-alkyl, in another embodiment R¹ is hydrogen. In one embodiment, a (C₁-C₄)-alkyl group representing R¹ or occurring in R¹ is a (C₁-C₃)-alkyl group, in another embodiment a (C₁-C₂)-alkyl group, in another embodiment a methyl group or methylene group, respectively. In one embodiment the number of substituents in a substituted phenyl group or heteroaryl group occurring in R¹ is 1, 2 or 3, in another embodiment 1 or 2, in another embodiment 1. In one embodiment, a phenyl group or heteroaryl group occurring in R¹ is unsubstituted.

In one embodiment of the invention, R² and R³ are independently of each other selected from the series consisting of halogen and (C₁-C₄)-alkyl-O—, in another embodiment from the series consisting of halogen and (C₁-C₄)-alkyl, in another embodiment from the series consisting of (C₁-C₄)-alkyl and (C₁-C₄)-alkyl-O—, in another embodiment from the series consisting of halogen, in another embodiment from the series consisting of (C₁-C₄)-alkyl-O—. In one embodiment, a (C₁-C₄)-alkyl group representing R² or R³ or occurring in R² or R³ is a (C₁-C₃)-alkyl group, in another embodiment a (C₁-C₂)-alkyl group, in another embodiment a methyl group. Groups R² and R³ can be present in any positions of the benzene rings within the two indole rings depicted in formula I, i.e. in any of positions 4, 5, 6 and 7 of any of the two indole rings. All carbon atoms in positions 4, 5, 6 and 7 of the two indole rings which do not carry a group R² or R³, respectively, carry a hydrogen atom. In one embodiment, groups R² and R³ are present in positions 5 and/or 6 of the indole rings.

In one embodiment of the invention, R⁴ is selected from the series consisting of (C₁-C₄)-alkyl, in another embodiment R⁴ is hydrogen. In one embodiment, a (C₁-C₄)-alkyl group representing R⁴ is a (C₁-C₃)-alkyl group, in another embodiment a (C₁-C₂)-alkyl group, in another embodiment a methyl group.

In one embodiment of the invention, R¹⁰ is selected from the series consisting of (C₁-C₆)-alkyl and Het¹, wherein (C₁-C₆)-alkyl is unsubstituted or substituted by R²⁰, and wherein Het¹ is unsubstituted or substituted by R²¹, in another embodiment from the series consisting of (C₁-C₆)-alkyl, wherein (C₁-C₆)-alkyl is unsubstituted or substituted by R²⁰, in another embodiment from the series consisting of Het¹, wherein Het¹ is unsubstituted or substituted by R²¹, and in these embodiments R¹¹ is an individual group as defined above or below, or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl. In another embodiment, R¹⁰ is selected from the series consisting of (C₁-C₆)-alkyl and Het¹, wherein (C₁-C₆)-alkyl is unsubstituted or substituted by R²⁰, and wherein Het¹ is unsubstituted or substituted by R²¹, in another embodiment from the series consisting of (C₁-C₆)-alkyl, wherein (C₁-C₆)-alkyl is unsubstituted or substituted by R²⁰, in another embodiment from the series consisting of Het¹, wherein Het¹ is unsubstituted or substituted by R²¹, and in these embodiments R¹¹ is an individual group as defined above or below, and R¹⁰ and R¹¹ together do not form a divalent group —(CH₂)ₘ—C(R²²)(R²³)—(CH₂)— or —(CH₂)ₘ—N(R²⁴)—(CH₂)ₙ—. In one embodiment, a (C₁-C₆)-alkyl group representing R¹⁰ is a (C₁-C₅)-alkyl group, in another embodiment a (C₁-C₄)-alkyl group, in another embodiment a (C₁-C₃)-alkyl group, in another embodiment a (C₁-C₂)-alkyl group, in another embodiment a methyl group or methylene group, respectively, in another embodiment a (C₂-C₆)-alkyl group, in another embodiment a (C₂-C₅)-alkyl group, in another embodiment a (C₂-C₄)-alkyl group, in another embodiment a (C₂-C₃)-alkyl group, wherein all these groups are unsubstituted or substituted by R²⁰. In one embodiment, an alkyl group representing R¹⁰ is unsubstituted, in another embodiment it is substituted by R²⁰. In one embodiment the number of substituents R²⁰ in a substituted alkyl group representing R¹⁰ is 1, 2 or 3, in another embodiment 1 or 2, in another embodiment 1. In one embodiment, the total number of optional substituents R²⁰ from the series consisting of H₂N—C(═NH)—S—, pyridinyl and Het² in an alkyl group representing R¹⁰ is 1 or 2, in another embodiment it is 1. Substituents R²⁰ in a substituted alkyl group representing R¹⁰ can be bonded to any carbon atom of the alkyl group. In one embodiment, in case of an alkyl group representing R¹⁰ which carries one substituent R²⁰, the substituent R²⁰ is bonded to the terminal carbon atom of a linear alkyl group, and the group R²⁰—(C₁-C₆)-alkyl- is, for example, any of the groups R²⁰—CH₂—, R²⁰—CH₂CH₂—, R²⁰—CH₂—CH₂—CH₂—, R²⁰—CH₂—CH₂—CH₂—CH₂—, R²⁰—CH₂—CH₂—CH₂—CH₂—CH₂— and R²⁰—CH₂—CH₂—CH₂—CH₂—CH₂—CH₂—. In one embodiment, a group Het¹ representing R¹⁰ is unsubstituted, in another embodiment it is substituted by R²¹. In one embodiment the number of substituents R²¹ in a substituted group Het¹ representing R¹⁰ is 1, 2 or 3, in another embodiment 1 or 2, in another embodiment 1. In one embodiment, the total number of optional substituents R²¹ in Het¹ from the series consisting of phenyl-(C₁-C₄)-alkyl- and pyridinyl-(C₁-C₄)-alkyl- is 1 or 2, in another embodiment it is 1. Substituents R²¹ in a substituted group Het¹ representing R¹⁰ can be bonded to any ring carbon atom and/or the ring nitrogen atom in Het¹. In one embodiment, the carbon atom via which Het¹ is bonded to the indole ring, does not carry a substituent R²¹. In another embodiment, the ring nitrogen atom in Het¹ carries a substituent R²¹.

In one embodiment of the invention, R¹¹ is selected from the series consisting of (C₁-C₄)-alkyl, in another embodiment R¹¹ is hydrogen. In one embodiment, a (C₁-C₄)-alkyl group representing R¹¹ is a (C₁-C₃)-alkyl group, in another embodiment a (C₁-C₂)-alkyl group, in another embodiment a methyl group.

If R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, a ring is formed which includes the ring nitrogen atom carrying R¹⁰ and the ring carbon atom carrying R¹¹, and which thus is fused to the indole ring of which the said ring nitrogen atom and ring carbon atom are ring members. Depending on the meaning of the integers m and n, the formed ring is a 5-membered, 6-membered or 7-membered ring. In the groups —(CH₂)ₘ—C(R²²)(R²³)—(CH₂)ₙ— and —(CH₂)ₘ—N(R²⁴)—(CH₂)ₙ— the moiety (CH₂)ₘ is bonded to the ring nitrogen atom carrying the group R¹⁰ in formula I, and the moiety (CH₂)ₙ is bonded to the ring carbon atom carrying the group R¹¹, this differentiation of the two moieties (CH₂)ₘ and (CH₂)ₙ being relevant in case m and n are different. In one embodiment of the invention, a divalent group formed by R¹⁰ and R¹¹ together is a divalent group selected from the series consisting of the groups —(CH₂)ₘ—C(R²²)(R²³)—(CH₂)ₙ—, in another embodiment it is a divalent group selected from the series consisting of —(CH₂)ₘ—N(R²⁴)—(CH₂)ₙ—, wherein in these groups the moieties (CH₂)ₘ and (CH₂)ₙ are unsubstituted or substituted by (C₁-C₄)-alkyl. In one embodiment, the total number of alkyl substituents in substituted moieties (CH₂)ₘ and (CH₂)ₙ is 1, 2, 3 or 4, in another embodiment 1, 2 or 3, in another embodiment 1 or 2, in another embodiment 1. In an individual CH₂ group the number of alkyl substituents can of course not exceed 2. In one embodiment, the moieties (CH₂)ₘ and (CH₂)ₙ are not substituted by (C₁-C₄)-alkyl. In one embodiment of the invention, R¹⁰ and R¹¹ are not individual groups as defined above, and they together are a divalent group selected from the series consisting of the groups —(CH₂)ₘ—C(R²²)(R²³)—(CH₂)ₙ— and —(CH₂)ₘ—N(R²⁴)—(CH₂)ₙ—, in another embodiment from the series consisting of the groups —(CH₂)ₘ—C(R²²)(R²³)—(CH₂)ₙ—, in another embodiment from the series consisting of —(CH₂)ₘ—N(R²⁴)—(CH₂)ₙ—, wherein the moieties (CH₂)ₘ and (CH₂)ₙ are unsubstituted or substituted by (C₁-C₄)-alkyl. If alkyl substituents are present in the moieties (CH₂)ₘ and (CH₂), they can be present in any one or more of the CH₂ groups, independently of any other CH₂ group, for example in the CH₂ group attached to the ring nitrogen atom of the indole ring and/or in the CH₂ group attached to the carbon atom in position 2 of the indole ring and/or in one or two of the CH₂ groups attached to the group C(R²²)(R²³) or the group N(R²⁴), respectively. In one embodiment, a (C₁-C₄)-alkyl substituent in the moieties (CH₂)ₘ and (CH₂)ₙ is a (C₁-C₃)-alkyl group, in another embodiment a (C₁-C₂)-alkyl group, in another embodiment a methyl group.

In one embodiment of the invention, R²⁰ is selected from the series consisting of R³¹—N(R³²)—, H₂N—C(═NH)—S—, pyridinyl and Het², in another embodiment from the series consisting of R³⁰—O—, R³¹—N(R³²)—, H₂N—C(═NH)—S— and Het², in another embodiment from the series consisting of R³⁰—O—, R³¹—N(R³²)—, pyridinyl and Het², in another embodiment from the series consisting of R³¹—N(R³²)—, H₂N—C(═NH)—S— and Het², in another embodiment from the series consisting of R³¹—N(R³²)—, pyridinyl and Het², in another embodiment from the series consisting of R³⁰—O—, R³¹—N(R³²)— and Het², in another embodiment from the series consisting of R³⁰—O— and R³¹—N(R³²)—, in another embodiment from the series consisting of R³¹—N(R³²)— and Het², in another embodiment R²⁰ is selected from the series consisting of R³¹—N(R³²)—, in another embodiment R²⁰ is selected from the series consisting of Het², wherein in all embodiments Het² is unsubstituted or substituted by R³³. In one embodiment, the number of substituents R³³ in a substituted group Het², which substituents can be present on ring carbon atoms and/or on ring nitrogen atoms in Het², is 1, 2, 3 or 4, in another embodiment 1, 2 or 3, in another embodiment 1 or 2, in another embodiment 1. In one embodiment, Het² is unsubstituted.

In one embodiment of the invention, R²¹ is selected from the series consisting of (C₁-C₄)-alkyl and pyridinyl-(C₁-C₄)-alkyl-, in another embodiment from the series consisting of (C₁-C₄)-alkyl and phenyl-(C₁-C₄)-alkyl-, in another embodiment from the series consisting of (C₁-C₄)-alkyl, in another embodiment from the series consisting of pyridinyl-(C₁-C₄)-alkyl-, wherein in all these embodiments phenyl and pyridinyl are unsubstituted or substituted by substituents from the series consisting of halogen, (C₁-C₄)-alkyl and (C₁-C₄)-alkyl-O—. In one embodiment the number of substituents in a substituted phenyl group or pyridinyl group occurring in R²¹ is 1, 2 or 3, in another embodiment 1 or 2, in another embodiment 1. In one embodiment, a phenyl group or pyridinyl group occurring in R²¹ is unsubstituted. In one embodiment, the substituents in a substituted phenyl or pyridinyl group occurring in R²¹ are selected from the series consisting of halogen and (C₁-C₄)-alkyl, in another embodiment from the series consisting of halogen. In one embodiment, a pyridinyl group occurring in R²¹ is selected from the series consisting of pyridin-2-yl and pyridin-3-yl, in another embodiment it is pyridin-2-yl, in another embodiment it is pyridin-3-yl, wherein all these pyridinyl groups are unsubstituted or substituted as indicated. In one embodiment, a (C₁-C₄)-alkyl group representing R²¹ or occurring in R²¹ is a (C₁-C₃)-alkyl group, in another embodiment a (C₁-C₂)-alkyl group, in another embodiment a methyl group or methylene group, respectively.

In one embodiment of the invention, R²² is selected from the series consisting of (C₁-C₄)-alkyl, in another embodiment R²² is hydrogen. In one embodiment, a (C₁-C₄)-alkyl group representing R²² is a (C₁-C₃)-alkyl group, in another embodiment a (C₁-C₂)-alkyl group, in another embodiment a methyl group.

In one embodiment of the invention, R²³ is selected from the series consisting of (C₁-C₄)-alkyl, R³¹—N(R³²)— and R³¹—N(R³²)—(C₁-C₄)-alkyl-, in another embodiment from the series consisting of R³¹—N(R³²)— and R³¹—N(R³²)—(C₁-C₄)-alkyl-, in another embodiment from the series consisting of R³¹—N(R³²)—, in another embodiment from the series consisting of R³¹—N(R³²)—(C₁-C₄)-alkyl-. In the group R³¹—N(R³²)—(C₁-C₄)-alkyl-, which is bonded to the remainder of the molecule via the (C₁-C₄)-alkyl moiety as is indicated by the terminal hyphen at the (C₁-C₄)-alkyl moiety, which in this group and likewise in all other groups composed of several moieties depicts the free bond via which the group is bonded, the moiety R³¹—N(R³²)— can be bonded to any carbon atom in the (C₁-C₄)-alkyl moiety. In one embodiment, the moiety R³¹—N(R³²)— in the group R³¹—N(R³²)—(C₁-C₄)-alkyl- representing R²³ is bonded to the terminal carbon atom of a linear alkyl group, and the group R³¹—N(R³²)—(C₁-C₄)-alkyl- is, for example, any of the groups R³¹—N(R³²)—CH₂—, R³¹—N(R³²)—CH₂— and R³¹—N(R³²)—CH₂—CH₂—CH₂—. In one embodiment, a (C₁-C₄)-alkyl group representing R²³ or occurring in R²³ is a (C₁-C₃)-alkyl group, in another embodiment a (C₁-C₂)-alkyl group, in another embodiment a methyl group or methylene group, respectively.

In one embodiment of the invention, R²⁴ is selected from the series consisting of hydrogen, (C₁-C₄)-alkyl and pyridinyl-(C₁-C₄)-alkyl-, in another embodiment from the series consisting of hydrogen, (C₁-C₄)-alkyl and R³¹—N(R³²)—(C₁-C₄)-alkyl-, in another embodiment from the series consisting of hydrogen and (C₁-C₄)-alkyl, in another embodiment from the series consisting of (C₁-C₄)-alkyl and R³¹—N(R³²)—(C₁-C₄)-alkyl-, in another embodiment from the series consisting of (C₁-C₄)-alkyl, and in another embodiment R²⁴ is hydrogen. In one embodiment, a (C₁-C₄)-alkyl group representing R²⁴ or occurring in R²⁴ is a (C₁-C₃)-alkyl group, in another embodiment a (C₁-C₂)-alkyl group, in another embodiment a methyl group or methylene group, respectively.

In one embodiment of the invention, any of the groups R³⁰, R³¹ and R³² is independently of each other group selected from the series consisting of (C₁-C₄)-alkyl, in another embodiment any of the groups R³⁰, R³¹ and R³² is independently of each other group hydrogen. In one embodiment, a (C₁-C₄)-alkyl group representing any of the groups R³⁰, R³¹ and R³² is independently of each other group a (C₁-C₃)-alkyl group, in another embodiment a (C₁-C₂)-alkyl group, in another embodiment a methyl group.

In one embodiment, R³³ is a (C₁-C₃)-alkyl group, in another embodiment a (C₁-C₂)-alkyl group, in another embodiment a methyl group.

Het¹ can be 4-membered, 5-membered, 6-membered or 7-membered, and thus be an azetidinyl group, pyrrolidinyl group, piperidinyl group or azepanyl group, which can all be bonded via any ring carbon atom to the ring nitrogen atom of the indole ring which carries the group R¹⁰. In one embodiment, Het¹ is 5-membered, 6-membered or 7-membered, in another embodiment it is 5-membered or 6-membered, in another embodiment it is 6-membered or 7-membered, and in another embodiment it is 6-membered and Het¹ thus is a piperidinyl group. In one embodiment of the invention, the group Het¹ is bonded via a ring carbon atom which is not adjacent to the ring nitrogen atom in Het¹. In one embodiment, a pyrrolidinyl group representing Het¹ is bonded via a carbon atom in position 3. In one embodiment, a piperidinyl group representing Het¹ is bonded via a carbon atom in position 3 or 4, in another embodiment via the carbon atom in position 4. In one embodiment, an azepanyl group representing Het¹ is bonded via a carbon atom in position 3 or 4, in another embodiment the carbon atom in position 4.

Het² can be 4-membered, 5-membered, 6-membered or 7-membered. Examples of heterocyclic groups from any one or more of which Het² is selected in one embodiment of the invention, are azetidinyl, pyrrolidinyl, imidazolidinyl, piperidinyl, perhydropyrimidinyl, piperazinyl, azepanyl, [1,3]diazepanyl and [1,4]diazepanyl, which can all be bonded via any ring carbon atom or any ring nitrogen atom. In one embodiment, Het² is bonded via a ring carbon atom, in another embodiment it is bonded via a ring nitrogen atom. In one embodiment, Het² comprises one ring nitrogen atom. In one embodiment, Het² is 4-membered, 5-membered or 6-membered, in another embodiment it is 5-membered, 6-membered or 7-membered, in another embodiment it is 5-membered or 6-membered, in another embodiment it is 6-membered or 7-membered, in another embodiment it is 5-membered, and in another embodiment it is 6-membered. In one embodiment, Het² is selected from the series consisting of pyrrolidinyl and piperidinyl, in another embodiment it is a pyrrolidinyl group, in another embodiment it is a piperidinyl group, and in another embodiment it is a piperazinyl group.

In one embodiment of the invention, heteroaryl is bonded via a ring carbon atom. In one embodiment, heteroaryl comprises two ring heteroatoms selected from the series consisting of N and S, in another embodiment one ring heteroatom selected from the series consisting of N, O and S, in another embodiment one ring heteroatom selected from the series consisting of N and S, in another embodiment heteroaryl is selected from the series consisting of thiophenyl and pyridinyl, and in another embodiment it is pyridinyl.

If any of the integers a and b is 0 (zero), no groups R² and R³, respectively, are present as substituents in the respective benzene ring within the indole rings depicted in formula I, and all carbon atoms in positions 4, 5, 6 and 7 of the respective indole ring carry a hydrogen atom. In one embodiment of the invention, any of the integers a and b is independently of each other selected from the series consisting of 0 and 1, in another embodiment any of the integers a and b is independently of each other 2, in another embodiment any of the integers a and b is independently of each other 1, and in another embodiment any of the integers a and b is independently of each other 0.

In one embodiment of the invention, the integer m, i.e. the number of CH₂ groups connecting the group C(R²²)(R²³) or the group N(R²⁴), respectively, to the ring nitrogen atom in the indole ring which carries the group R¹⁰ in formula I, is 2, in another embodiment it is 1. In one embodiment of the invention the integer n, i.e. the number of CH₂ groups connecting the group C(R²²)(R²³) or the group N(R²⁴), respectively, to the ring carbon atom in the indole ring which carries the group R¹¹ in formula I, is 2, in another embodiment it is 1. In one embodiment, m is selected from the series consisting of 1 and 2, and n is 1, in another embodiment m is 2 and n is 1.

In embodiments, the present invention includes all compounds of the formula I in which any one or more structural elements such as groups, residues, substituents and integers are defined as in any of the specified embodiments or definitions of the elements, or have one or more of the specific meanings which are mentioned herein as examples of elements, wherein all combinations of one or more definitions of compounds or elements and/or specified embodiments and/or specific meanings of elements are included as disclosed items. Also with respect to all such compounds of the formula I, all their stereoisomeric forms and mixtures of stereoisomeric forms in any ratio, and their physiologically acceptable salts are included in embodiments of the present invention.

### Further Embodiments of Compounds Having the Formula I

A. In an embodiment, in the compound of formula I: R¹ is selected from the series consisting of hydrogen, (C₁-C₄)-alkyl and phenyl-(C₁-C₄)-alkyl-, wherein phenyl is unsubstituted or substituted by substituents from the series consisting of halogen, (C₁-C₄)-alkyl and (C₁-C₄)-alkyl-O—;

R² and R³ are independently of each other selected from the series consisting of halogen, (C₁-C₄)-alkyl and (C₁-C₄)-alkyl-O—;

R⁴ is selected from the series consisting of hydrogen and (C₁-C₄)-alkyl;

R¹⁰ is selected from the series consisting of (C₁-C₆)-alkyl and Het¹, wherein (C₁-C₆)-alkyl is unsubstituted or substituted by R²⁰, and wherein Het¹ is unsubstituted or substituted by R²¹;

R¹¹ is selected from the series consisting of hydrogen and (C₁-C₄)-alkyl,

or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl;

R²⁰ is selected from the series consisting of R³¹—N(R³²)—, H₂N—C(═NH)—S—, pyridinyl and Het², wherein Het² is unsubstituted or substituted by R³³;

R²¹ is selected from the series consisting of (C₁-C₄)-alkyl, phenyl-(C₁-C₄)-alkyl- and pyridinyl-(C₁-C₄)-alkyl-, wherein phenyl and pyridinyl are unsubstituted or substituted by substituents from the series consisting of halogen, (C₁-C₄)-alkyl and (C₁-C₄)-alkyl-O—;

R²² is selected from the series consisting of hydrogen and (C₁-C₄)-alkyl;

R²³ is selected from the series consisting of hydrogen, (C₁-C₄)-alkyl, R³¹—N(R³²)— and R³¹—N(R³²)—(C₁-C₄)-alkyl-;

R²⁴ is selected from the series consisting of hydrogen, (C₁-C₄)-alkyl, pyridinyl-(C₁-C₄)-alkyl- and R³¹—N(R³²)—(C₁-C₄)-alkyl-;

R³¹ and R³² are independently of each other selected from the series consisting of hydrogen and (C₁-C₄)-alkyl;

R³³ is selected from the series consisting of (C₁-C₄)-alkyl;

Het¹ is a 4-membered to 7-membered, monocyclic, saturated heterocycle comprising one ring nitrogen atom, which is bonded via a ring carbon atom;

Het² is a 4-membered to 7-membered, monocyclic, saturated heterocycle comprising one or two ring nitrogen atoms, which is bonded via a ring carbon atom or a ring nitrogen atom;

a and b are independently of each other selected from the series consisting of 0 and 1;

m and n are independently of each other selected from the series consisting of 1 and 2.

B. In an embodiment, in the compound of formula I: R¹ is selected from the series consisting of hydrogen, (C₁-C₄)-alkyl and phenyl-(C₁-C₄)-alkyl-;

R² and R³ are independently of each other selected from the series consisting of halogen, (C₁-C₄)-alkyl and (C₁-C₄)-alkyl-O—;

R⁴ is selected from the series consisting of hydrogen and (C₁-C₄)-alkyl;

R¹⁰ is selected from the series consisting of (C₁-C₆)-alkyl and Het¹, wherein (C₁-C₆)-alkyl is unsubstituted or substituted by R²⁰, and wherein Het¹ is unsubstituted or substituted by R²¹;

R¹¹ is selected from the series consisting of hydrogen and (C₁-C₄)-alkyl,

or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl;

R²⁰ is selected from the series consisting of R³¹—N(R³²)—, H₂N—C(═NH)—S— and Het², wherein Het² is unsubstituted or substituted by R³³;

R²¹ is selected from the series consisting of (C₁-C₄)-alkyl and pyridinyl-(C₁-C₄)-alkyl-, wherein pyridinyl is unsubstituted or substituted by substituents from the series consisting of halogen, (C₁-C₄)-alkyl and (C₁-C₄)-alkyl-O—;

R²² is hydrogen;

R²³ is selected from the series consisting of hydrogen, (C₁-C₄)-alkyl, R³¹—N(R³²)— and

R³¹—N(R³²)—(C₁-C₄)-alkyl-;

R²⁴ is selected from the series consisting of hydrogen, (C₁-C₄)-alkyl and pyridinyl-(C₁-C₄)-alkyl-;

R³¹ and R³² are independently of each other selected from the series consisting of hydrogen and (C₁-C₄)-alkyl;

R³³ is selected from the series consisting of (C₁-C₄)-alkyl;

Het¹ is a 5-membered or 6-membered, monocyclic, saturated heterocycle comprising one ring nitrogen atom, which is bonded via a ring carbon atom;

Het² is a 4-membered to 6-membered, monocyclic, saturated heterocycle comprising one or two ring nitrogen atoms, which is bonded via a ring carbon atom or a ring nitrogen atom;

a and b are independently of each other selected from the series consisting of 0 and 1;

m and n are independently of each other selected from the series consisting of 1 and 2.

C. In an embodiment, in the compound of formula I: R¹ is hydrogen;

R⁴ is selected from the series consisting of hydrogen and (C₁-C₄)-alkyl;

R¹⁰ is selected from the series consisting of (C₁-C₄)-alkyl and Het¹, wherein (C₁-C₄)-alkyl is unsubstituted or substituted by R²⁰, and wherein Het¹ is unsubstituted or substituted by R²¹;

R¹¹ is hydrogen,

or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—;

R²⁰ is selected from the series consisting of R³¹—N(R³²)—, H₂N—C(═NH)—S— and Het², wherein Het² is unsubstituted or substituted by R³³;

R²¹ is selected from the series consisting of pyridinyl-(C₁-C₄)-alkyl-;

R²² is hydrogen;

R²³ is selected from the series consisting of R³¹—N(R³²)— and R³¹—N(R³²)—(C₁-C₄)-alkyl-R²⁴ is selected from the series consisting of hydrogen and (C₁-C₄)-alkyl;

R³¹ and R³² are independently of each other selected from the series consisting of hydrogen and (C₁-C₄)-alkyl;

R³³ is selected from the series consisting of (C₁-C₄)-alkyl;

Het¹ is a 5-membered or 6-membered, monocyclic, saturated heterocycle comprising one ring nitrogen atom, which is bonded via a ring carbon atom;

Het² is a 4-membered to 6-membered, monocyclic, saturated heterocycle comprising one or two ring nitrogen atoms, which is bonded via a ring carbon atom or a ring nitrogen atom;

a and b are 0;

m is 2 and n is 1.

D. In an embodiment, in the compound of formula I: R¹ is hydrogen;

R⁴ is selected from the series consisting of hydrogen and (C₁-C₄)-alkyl;

R¹⁰ is selected from the series consisting of (C₁-C₄)-alkyl, wherein (C₁-C₄)-alkyl is unsubstituted or substituted by R²⁰;

R¹¹ is hydrogen,

or R¹⁰ and R¹¹ together are a divalent group selected from the series consisting of the groups —(CH₂)ₘ—C(R²²)(R²³)—(CH₂)ₙ—;

R²⁰ is selected from the series consisting of R³¹—N(R³²)—, H₂N—C(═NH)—S— and Het², wherein Het² is unsubstituted or substituted by R³³;

R²² is hydrogen;

R²³ is selected from the series consisting of R³¹—N(R³²)— and R³¹—N(R³²)—(C₁-C₄)-alkyl-;

R³¹ and R³² are independently of each other selected from the series consisting of hydrogen and (C₁-C₄)-alkyl;

R³³ is selected from the series consisting of (C₁-C₄)-alkyl;

Het² is a 5-membered to 6-membered, monocyclic, saturated heterocycle comprising one or two ring nitrogen atoms, which is bonded via a ring carbon atom or a ring nitrogen atom;

a and b are 0;

m is 2 and n is 1.

E. In an embodiment, in the compound of formula I: R¹ is hydrogen;

R⁴ is selected from the series consisting of hydrogen and (C₁-C₄)-alkyl;

R¹⁰ is selected from the series consisting of (C₁-C₄)-alkyl, wherein (C₁-C₄)-alkyl is unsubstituted or substituted by R²⁰;

R¹¹ is hydrogen,

or R¹⁰ and R¹¹ together are a divalent group selected from the series consisting of the groups —(CH₂)ₘ—C(R²²)(R²³)—(CH₂)ₙ—;

R²⁰ is selected from the series consisting of R³¹—N(R³²)— and Het², wherein Het² is unsubstituted or substituted by R³³;

R²² is hydrogen;

R²³ is selected from the series consisting of R³¹—N(R³²)—(C₁-C₄)-alkyl-;

R³¹ and R³² are independently of each other selected from the series consisting of hydrogen and (C₁-C₄)-alkyl;

R³³ is selected from the series consisting of (C₁-C₄)-alkyl;

Het² is a 5-membered to 6-membered, monocyclic, saturated heterocycle comprising one ring nitrogen atom, which is bonded via a ring carbon atom;

a and b are 0;

m is 2 and n is 1.

F. In an embodiment, in the compound of formula I: R¹ is hydrogen;

R⁴ is selected from the series consisting of (C₁-C₄)-alkyl;

R¹⁰ and R¹¹ together are a divalent group selected from the series consisting of the groups —(CH₂)ₘ—C(R²²)(R²³)—(CH₂)ₙ—;

R²² is hydrogen;

R²³ is selected from the series consisting of R³¹—N(R³²)—(C₁-C₄)-alkyl-;

R³¹ and R³² are independently of each other selected from the series consisting of hydrogen and (C₁-C₄)-alkyl;

a and b are 0;

m is 2 and n is 1.

Specific examples of compounds of the formula I, which can generally be named as optionally 1-R¹-substituted, 3-(optionally substituted indol-3-yl)-substituted, 4-(optionally substituted indol-3-yl)-substituted pyrrole-2,5-diones, or as optionally N—R¹-substituted, 2-(optionally substituted indol-3-yl)-substituted, 3-(optionally substituted indol-3-yl)-substituted maleimides, or in another manner according to common procedures of chemical nomenclature, are the following compounds, from any one or more of which the compound of the formula I is selected in further embodiments of the invention:

3-(8-aminomethyl-6,7,8,9-tetrahydro-pyrido[1,2-a]indol-10-yl)-4-(1-methyl-1H-indol-3-yl)-pyrrole-2,5-dione, which is also known as Bisindolylmaleimide X and BIM X and Ro 31-8425, including all its stereoisomeric forms, such as the R isomer and the S isomer, and mixtures of stereoisomeric forms in any ratio, such as the racemate, and the physiologically acceptable salts thereof, such as 3-(8-aminomethyl-6,7,8,9-tetrahydro-pyrido[1,2-a]indol-10-yl)-4-(1-methyl-1H-indol-3-yl)-pyrrole-2,5-dione hydrochloride;

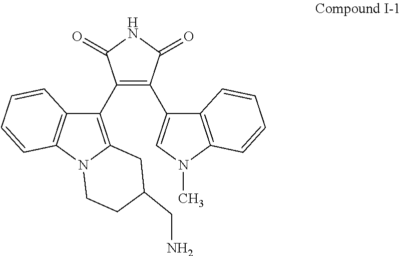 [SMILES: Cn1cc(C2=C(c3c4n(c5ccccc35)CCC(CN)C4)C(=O)NC2=O)c2ccccc21]

The compound of formula I-1 can be made as a salt such as the hydrochloride salt which is designated herein as compound 1929, and as a hydrate of the hydrochloride salt which is designated herein as compound 1919.

3-(8-dimethylaminomethyl-6,7,8,9-tetrahydro-pyrido[1,2-a]indol-10-yl)-4-(1-methyl-1H-indol-3-yl)-pyrrole-2,5-dione, which is also known as Bisindolylmaleimide XI and BIM XI and Ro 32-0432, including all its stereoisomeric forms, such as the R isomer and the S isomer, and mixtures of stereoisomeric forms in any ratio, such as the racemate, and the physiologically acceptable salts thereof, such as 3-(8-dimethylaminomethyl-6,7,8,9-tetrahydro-pyrido[1,2-a]indol-10-yl)-4-(1-methyl-1H-indol-3-yl)-pyrrole-2,5-dione hydrochloride;

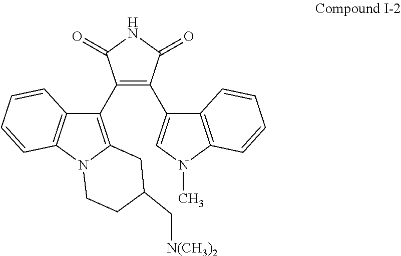 [SMILES: CN(C)CC1CCn2c(c(C3=C(c4cn(C)c5ccccc45)C(=O)NC3=O)c3ccccc32)C1]

The compound of formula I-2 can be made as a salt such as the hydrochloride salt which is designated herein as compound 1933.

3,4-bis-(1H-indol-3-yl)-1-methyl-pyrrole-2,5-dione, which is also known as Bisindolylmaleimide V and BIM V and Ro 31-6045;

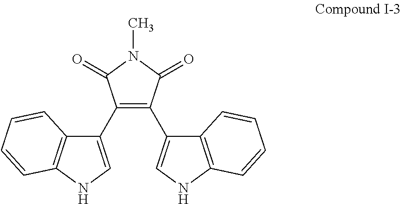 [SMILES: CN1C(=O)C(c2c[nH]c3ccccc23)=C(c2c[nH]c3ccccc23)C1=O]

1-benzyl-3,4-bis-(1H-indol-3-yl)-pyrrole-2,5-dione;

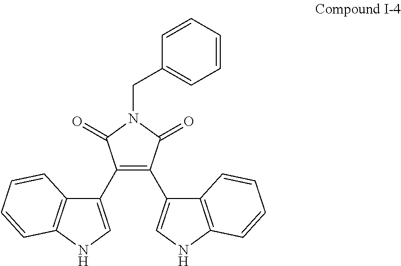 [SMILES: O=C1C(c2c[nH]c3ccccc23)=C(c2c[nH]c3ccccc23)C(=O)N1Cc1ccccc1]

The compound of formula I-4 is designated herein as compound 1921.

3-(1H-indol-3-yl)-1-methyl-4-(1-methyl-1H-indol-3-yl)-pyrrole-2,5-dione;

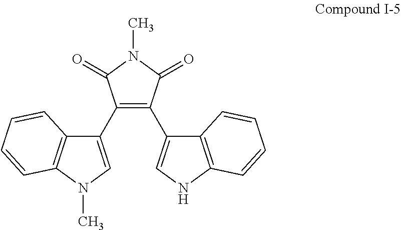 [SMILES: CN1C(=O)C(c2c[nH]c3ccccc23)=C(c2cn(C)c3ccccc23)C1=O]

The compound of formula I-5 is designated herein as compound 1905.

3-(1-methyl-1H-indol-3-yl)-4-[1-(1-pyridin-2-ylmethyl-piperidin-4-yl)-1H-indol-3-yl]-pyrrole-2,5-dione, which is also known as Enzastaurin and LY 317615, and the physiologically acceptable salts thereof, such as 3-(1-methyl-1H-indol-3-yl)-4-[1-(1-pyridin-2-ylmethyl-piperidin-4-yl)-1H-indol-3-yl]-pyrrole-2,5-dione hydrochloride and 3-(1-methyl-1H-indol-3-yl)-4-[1-(1-pyridin-2-ylmethyl-piperidin-4-yl)-1H-indol-3-yl]-pyrrole-2,5-dione dihydrochloride;

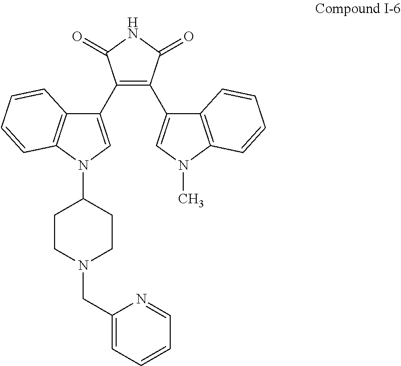 [SMILES: Cn1cc(C2=C(c3cn(C4CCN(Cc5ccccn5)CC4)c4ccccc34)C(=O)NC2=O)c2ccccc21]

The compound of formula I-6 is designated herein as compound 1906.

3,4-bis-(1H-indol-3-yl)-pyrrole-2,5-dione, which is also known as Bisindolylmaleimide IV and BIM IV;

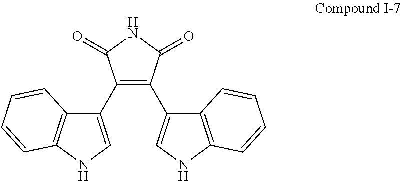 [SMILES: O=C1NC(=O)C(c2c[nH]c3ccccc23)=C1c1c[nH]c2ccccc12]

3-(1H-indol-3-yl)-4-(1-methyl-1H-indol-3-yl)-pyrrole-2,5-dione;

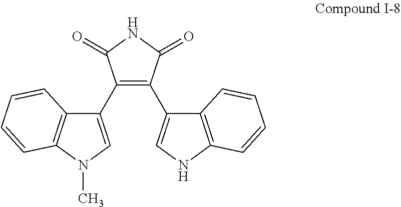 [SMILES: Cn1cc(C2=C(c3c[nH]c4ccccc34)C(=O)NC2=O)c2ccccc21]

3-[1-(3-dimethylamino-propyl)-1H-indol-3-yl]-4-(1H-indol-3-yl)-pyrrole-2,5-dione, which is also known as Bisindolylmaleimide I and BIM I, and the physiologically acceptable salts thereof, such as 3-[1-(3-dimethylamino-propyl)-1H-indol-3-yl]-4-(1H-indol-3-yl)-pyrrole-2,5-dione hydrochloride;

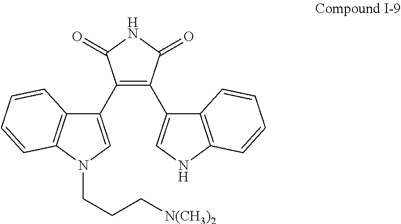 [SMILES: CN(C)CCCn1cc(C2=C(c3c[nH]c4ccccc34)C(=O)NC2=O)c2ccccc21]

The compound of formula I-9 is designated herein as compound 1934.

3,4-bis-(1-methyl-1H-indol-3-yl)-pyrrole-2,5-dione;

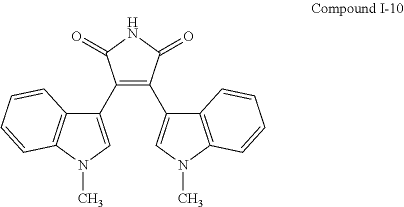 [SMILES: Cn1cc(C2=C(c3cn(C)c4ccccc34)C(=O)NC2=O)c2ccccc21]

The compound of formula I-10 is designated herein as compound 1912.

3-[1-(3-amidinothio-propyl)-1H-indol-3-yl]-4-(1-methyl-1H-indol-3-yl)-pyrrole-2,5-dione, which can also be named as S-(3-{3-[4-(1-methyl-1H-indol-3-yl)-2,5-dioxo-2,5-dihydro-1H-pyrrol-3-yl]-indol-1-yl}-propyl)-isothiourea or as carbamimidothioic acid 3-{3-[2,5-dihydro-4-(1-methyl-1H-indol-3-yl)-2,5-dioxo-1H-pyrrol-3-yl}-1H-indol-1-yl]propyl ester, for example, and which is also known as Bisindolylmaleimide IX and BIM IX and Ro 31-8220, and the physiologically acceptable salts thereof, such as 3-[1-(3-amidinothio-propyl)-1H-indol-3-yl]-4-(1-methyl-1H-indol-3-yl)-pyrrole-2,5-dione methanesulfonate;

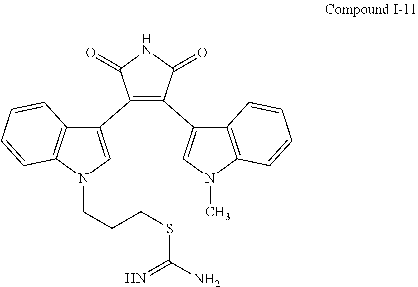 [SMILES: Cn1cc(C2=C(c3cn(CCCSC(=N)N)c4ccccc34)C(=O)NC2=O)c2ccccc21]

3-[1-(3-amino-propyl)-1H-indol-3-yl]-4-(1H-indol-3-yl)-pyrrole-2,5-dione, which is also known as Bisindolylmaleimide III and BIM III, and the physiologically acceptable salts thereof, such as 3-[1-(3-amino-propyl)-1H-indol-3-yl]-4-(1H-indol-3-yl)-pyrrole-2,5-dione hydrochloride;

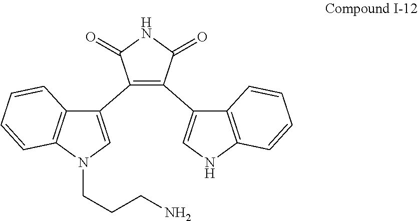 [SMILES: NCCCn1cc(C2=C(c3c[nH]c4ccccc34)C(=O)NC2=O)c2ccccc21]

3-[1-(3-amino-propyl)-1H-indol-3-yl]-4-(1-methyl-1H-indol-3-yl)-pyrrole-2,5-dione, which is also known as Bisindolylmaleimide VIII and BIM VIII and Ro 31-7549, and the physiologically acceptable salts thereof, such as 3-[1-(3-amino-propyl)-1H-indol-3-yl]-4-(1-methyl-1H-indol-3-yl)-pyrrole-2,5-dione hydrochloride and 3-[1-(3-amino-propyl)-1H-indol-3-yl]-4-(1-methyl-1H-indol-3-yl)-pyrrole-2,5-dione acetate;

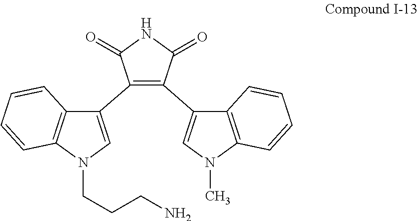 [SMILES: Cn1cc(C2=C(c3cn(CCCN)c4ccccc34)C(=O)NC2=O)c2ccccc21]

3-(1H-indol-3-yl)-4-{1-[2-(1-methyl-pyrrolidin-2-yl)-ethyl]-1H-indol-3-yl}-pyrrole-2,5-dione, which is also known as Bisindolylmaleimide II and BIM II, including all its stereoisomeric forms, such as the R isomer and the S isomer, and mixtures of stereoisomeric forms in any ratio, such as the racemate, and the physiologically acceptable salts thereof;

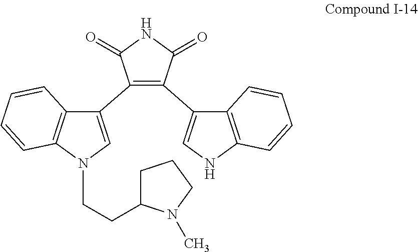 [SMILES: CN1CCCC1CCn1cc(C2=C(c3c[nH]c4ccccc34)C(=O)NC2=O)c2ccccc21]

The compound of formula I-14 is designated herein as compound 1928.

3-[1-(3-dimethylamino-propyl)-5-methoxy-1H-indol-3-yl]-4-(1H-indol-3-yl)-pyrrole-2,5-dione, and the physiologically acceptable salts thereof;

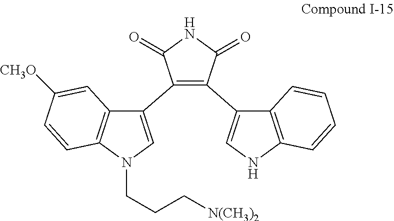

3-(6-bromo-1H-indol-3-yl)-4-(1-methyl-1H-indol-3-yl)-pyrrole-2,5-dione;

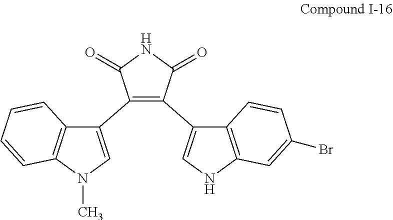 [SMILES: Cn1cc(C2=C(c3c[nH]c4cc(Br)ccc34)C(=O)NC2=O)c2ccccc21]

3-[1-(3-hydroxy-propyl)-1H-indol-3-yl)-4-(1-methyl-1H-indol-3-yl)-pyrrole-2,5-dione;

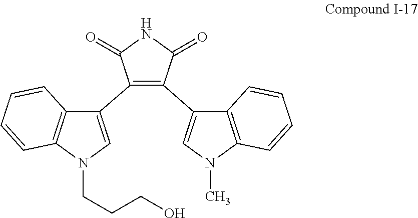 [SMILES: Cn1cc(C2=C(c3cn(CCCO)c4ccccc34)C(=O)NC2=O)c2ccccc21]

3-(1H-indol-3-yl)-4-[1-(2-piperidin-2-yl-ethyl)-1H-indol-3-yl]-pyrrole-2,5-dione, which is also known as Bisindolylmaleimide VI and BIM VI, including all its stereoisomeric forms, such as the R isomer and the S isomer, and mixtures of stereoisomeric forms in any ratio, such as the racemate, and the physiologically acceptable salts thereof

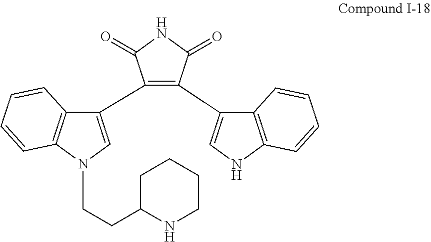 [SMILES: O=C1NC(=O)C(c2cn(CCC3CCCCN3)c3ccccc23)=C1c1c[nH]c2ccccc12]

3-(1H-indol-3-yl)-4-[1-(3-piperazin-1-yl-propyl)-1H-indol-3-yl]-pyrrole-2,5-dione, which is also known as Bisindolylmaleimide VII and BIM VII, and the physiologically acceptable salts thereof

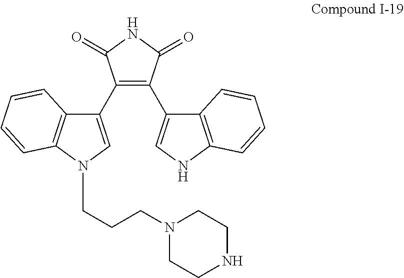 [SMILES: O=C1NC(=O)C(c2cn(CCCN3CCNCC3)c3ccccc23)=C1c1c[nH]c2ccccc12]

For example, in one embodiment of the invention the compound of the formula I is selected from
3-(8-aminomethyl-6,7,8,9-tetrahydro-pyrido[1,2-a]indol-10-yl)-4-(1-methyl-1H-indol-3-yl)-pyrrole-2,5-dione,
3-(8-dimethylaminomethyl-6,7,8,9-tetrahydro-pyrido[1,2-a]indol-10-yl)-4-(1-methyl-1H-indol-3-yl)-pyrrole-2,5-dione,
3,4-bis-(1H-indol-3-yl)-1-methyl-pyrrole-2,5-dione,
1-benzyl-3,4-bis-(1H-indol-3-yl)-pyrrole-2,5-dione,
3-(1H-indol-3-yl)-1-methyl-4-(1-methyl-1H-indol-3-yl)-pyrrole-2,5-dione,
3-(1-methyl-1H-indol-3-yl)-4-[1-(1-pyridin-2-ylmethyl-piperidin-4-yl)-1H-indol-3-yl]-pyrrole-2,5-dione,
3,4-bis-(1H-indol-3-yl)-pyrrole-2,5-dione,
3-(1H-indol-3-yl)-4-(1-methyl-1H-indol-3-yl)-pyrrole-2,5-dione,
3-[1-(3-dimethylamino-propyl)-1H-indol-3-yl]-4-(1H-indol-3-yl)-pyrrole-2,5-dione,
3,4-bis-(1-methyl-1H-indol-3-yl)-pyrrole-2,5-dione,
3-[1-(3-amidinothio-propyl)-1H-indol-3-yl]-4-(1-methyl-1H-indol-3-yl)-pyrrole-2,5-dione,
3-[1-(3-amino-propyl)-1H-indol-3-yl]-4-(1H-indol-3-yl)-pyrrole-2,5-dione,
3-[1-(3-amino-propyl)-1H-indol-3-yl]-4-(1-methyl-1H-indol-3-yl)-pyrrole-2,5-dione,
3-(1H-indol-3-yl)-4-{1-[2-(1-methyl-pyrrolidin-2-yl)-ethyl]-1H-indol-3H-indol-3-yl}-pyrrole-2,5-dione, and
3-[1-(3-dimethylamino-propyl)-5-methoxy-1H-indol-3-yl]-4-(1H-indol-3-yl)-pyrrole-2,5-dione,

wherein a compound is present in any of its stereoisomeric forms or a mixture of stereoisomeric forms in any ratio, or a physiologically acceptable salt thereof, if applicable.

In another embodiment of the invention the compound of the formula I is selected from
3-(8-aminomethyl-6,7,8,9-tetrahydro-pyrido[1,2-a]indol-10-yl)-4-(1-methyl-1H-indol-3-yl)-pyrrole-2,5-dione,
3-(8-dimethylaminomethyl-6,7,8,9-tetrahydro-pyrido[1,2-a]indol-10-yl)-4-(1-methyl-1H-indol-3-yl)-pyrrole-2,5-dione,
1-benzyl-3,4-bis-(1H-indol-3-yl)-pyrrole-2,5-dione,
3-(1-methyl-1H-indol-3-yl)-4-[1-(1-pyridin-2-ylmethyl-piperidin-4-yl)-1H-indol-3-yl]-pyrrole-2,5-dione,
3-(1H-indol-3-yl)-4-(1-methyl-1H-indol-3-yl)-pyrrole-2,5-dione,
3-[1-(3-dimethylamino-propyl)-1H-indol-3-yl]-4-(1H-indol-3-yl)-pyrrole-2,5-dione,
3,4-bis-(1-methyl-1H-indol-3-yl)-pyrrole-2,5-dione,
3-[1-(3-amino-propyl)-1H-indol-3-yl]-4-(1H-indol-3-yl)-pyrrole-2,5-dione,
3-[1-(3-amino-propyl)-1H-indol-3-yl]-4-(1-methyl-1H-indol-3-yl)-pyrrole-2,5-dione, and
3-(1H-indol-3-yl)-4-{1-[2-(1-methyl-pyrrolidin-2-yl)-ethyl]-1H-indol-3H-indol-3-yl}-pyrrole-2,5-dione,

wherein a compound is present in any of its stereoisomeric forms or a mixture of stereoisomeric forms in any ratio, or a physiologically acceptable salt thereof, if applicable.

In another embodiment of the invention the compound of the formula I is selected from
3-(8-aminomethyl-6,7,8,9-tetrahydro-pyrido[1,2-a]indol-10-yl)-4-(1-methyl-1H-indol-3-yl)-pyrrole-2,5-dione,
3-(8-dimethylaminomethyl-6,7,8,9-tetrahydro-pyrido[1,2-a]indol-10-yl)-4-(1-methyl-1H-indol-3-yl)-pyrrole-2,5-dione,
3-(1-methyl-1H-indol-3-yl)-4-[1-(1-pyridin-2-ylmethyl-piperidin-4-yl)-1H-indol-3-yl]-pyrrole-2,5-dione,
3-[1-(3-dimethylamino-propyl)-1H-indol-3-yl]-4-(1H-indol-3-yl)-pyrrole-2,5-dione,
3,4-bis-(1-methyl-1H-indol-3-yl)-pyrrole-2,5-dione,
3-[1-(3-amino-propyl)-1H-indol-3-yl]-4-(1H-indol-3-yl)-pyrrole-2,5-dione,
3-[1-(3-amino-propyl)-1H-indol-3-yl]-4-(1-methyl-1H-indol-3-yl)-pyrrole-2,5-dione, and
3-(1H-indol-3-yl)-4-{1-[2-(1-methyl-pyrrolidin-2-yl)-ethyl]-1H-indol-3H-indol-3-yl}-pyrrole-2,5-dione,

wherein a compound is present in any of its stereoisomeric forms or a mixture of stereoisomeric forms in any ratio, or a physiologically acceptable salt thereof, if applicable.

In another embodiment of the invention the compound of the formula I is selected from
3-(8-aminomethyl-6,7,8,9-tetrahydro-pyrido[1,2-a]indol-10-yl)-4-(1-methyl-1H-indol-3-yl)-pyrrole-2,5-dione,
3-(8-dimethylaminomethyl-6,7,8,9-tetrahydro-pyrido[1,2-a]indol-10-yl)-4-(1-methyl-1H-indol-3-yl)-pyrrole-2,5-dione,
3,4-bis-(1-methyl-1H-indol-3-yl)-pyrrole-2,5-dione, and
3-(1H-indol-3-yl)-4-{1-[2-(1-methyl-pyrrolidin-2-yl)-ethyl]-1H-indol-3-yl}-pyrrole-2,5-dione,

wherein a compound is present in any of its stereoisomeric forms or a mixture of stereoisomeric forms in any ratio, or a physiologically acceptable salt thereof, if applicable.

In another embodiment of the invention the compound of the formula I is selected from
3-(8-aminomethyl-6,7,8,9-tetrahydro-pyrido[1,2-a]indol-10-yl)-4-(1-methyl-1H-indol-3-yl)-pyrrole-2,5-dione, and
3-(8-dimethylaminomethyl-6,7,8,9-tetrahydro-pyrido[1,2-a]indol-10-yl)-4-(1-methyl-1H-indol-3-yl)-pyrrole-2,5-dione,

wherein a compound is present in any of its stereoisomeric forms or a mixture of stereoisomeric forms in any ratio, or a physiologically acceptable salt thereof.

In another embodiment of the invention the compound of the formula I is 3-(8-aminomethyl-6,7,8,9-tetrahydro-pyrido[1,2-a]indol-10-yl)-4-(1-methyl-1H-indol-3-yl)-pyrrole-2,5-dione, in any of its stereoisomeric forms or a mixture of stereoisomeric forms in any ratio, or a physiologically acceptable salt thereof.

Various compounds of the formula I have already been described in the literature, such as the exemplary specific compounds of the formula I specified above, and many of them are commercially available. The compounds of the formula I can be synthesized according to, or analogously to, various procedures which are described in the literature, such as in U.S. Pat. No. 5,721,245; U.S. Pat. No. 5,380,746; U.S. Pat. No. 5,545,636; U.S. Pat. No. 5,057,614; EP 1057484; U.S. Pat. No. 8,440,656; WO 95/17182; Bit, R. A. et al., J. Med. Chem. 1993, 36, 21-29; Davis, P. D. et al., J. Med. Chem. 1992, 35, 177-184; Davis, P. D. et al., J. Med. Chem. 1992, 35, 994-1001; Toullec, D. et al., J. Biol. Chem. 1991, 266, 15771-15781; Lu, Q. et al., Bioorg. Med. Chem. Lett. 2008, 19, 2399-2403; Sanchez-Martinez, C. et al., Bioorg. Med. Chem Lett. 2003, 13, 3841-3846; for example, and which are well known to a person skilled in the art.

In an embodiment, the contacting of MSCs with a compound composition as described herein may occur prior to or after cell cryopreservation.

In an embodiment of the invention, provided also are novel compounds of the formula I as such, in any of its stereoisomeric forms or a mixture of stereoisomeric forms in any ratio, and a physiologically acceptable salt thereof, if applicable.

In an embodiment, a compound as described herein is a prodrug form of a compound, wherein the compound is metabolized or modified during the pretreatment of MSCs with the compound. The compound is transformed into a successor compound which is an active compound and is capable of inducing an improved homing function of MSCs.

### Example 1

### Improved Homing of MSCs Treated with Small Molecule Compounds

### Introduction.

In this example, we demonstrate the improved homing of MSCs treated with a small molecule compound of the formula I. Also, the example describes a small molecule screen for targeted delivery of systemically infused cells. This disclosure contributes significant advances in developing technology to improve homing. This leads directly to results of beneficial therapeutic methods and compositions, particularly in conditions involving inflammation such as caused by injury and disease.

### Overview.

Poor homing to inflamed sites may limit the success of exogenous cell-based therapy. We screened a library of signal transduction modulators to identify hits that increase mesenchymal stem cell (MSC) surface expression of a key homing ligand, CD11a. Pretreatment of MSCs, by exposure to identified hits of certain active compounds, increased MSC firm adhesion to an E-selectin-coated substrate and enabled targeted MSC delivery to inflamed sites leading to a heightened anti-inflammatory response, following systemic administration.

### BACKGROUND

While exogenous cell therapy is a promising approach for treating a wide array of tragic diseases, a major challenge is that the majority of cell types exhibit poor homing to disease sites following systemic transplantation; this limits their therapeutic impact. Therefore, engineering cells to endow them with targeting potential to desired sites presents an attractive strategy. However, to date no approaches exist for using medium to high throughput small molecule screening to identify agents that promote cell homing following a simple pretreatment regimen. In this study, we report for the first time a multi-step process that includes a throughput screen to detect small molecules that improve targeting of systemically infused mesenchymal stromal cells to sites of inflammation. Furthermore, we report the discovery of methods and compositions useful for generating MSCs with improved homing and using MSCs including for therapeutic purposes. Mesenchymal stromal cells, otherwise known as mesenchymal stem cells (MSCs), are promising candidates for cell therapy given their pleotropic properties. Specifically, MSC can be readily isolated from bone marrow, fat and other adult tissues, and can be expanded under ex-vivo conditions to obtain a sufficient quantity for transplantation. They are considered immune-evasive, and their multi-lineage differentiation potential as well as potent immunomodulatory properties prompted their exploration in over 350 clinical trials as potential treatment for multiple diseases, including GvHD, diabetes, multiple sclerosis, and cardiovascular diseases. While results from pre-clinical animal studies have been encouraging and hundreds of millions of allogeneic MSCs can be safely administered systemically to patients, clinical trials have produced mixed results. The translational potential of therapy with MSCs has not yet been fully realized.

The majority of clinical trials involve systemic infusion of MSCs, yet MSCs exhibit poor homing to diseased or damaged tissues. Key ligands of the classical cell homing cascade that mediate dynamic cell interactions with activated endothelium are generally minimally expressed by MSCs or lost during in vitro expansion. Modifying MSCs with homing ligands via DNA transfection, surface modification and cytokine pretreatment improves their targeting to diseased sites. However, such approaches could be challenging to scale in a cost effective manner and include safety concerns in the case of viral modifications. Although several high throughput screens of bioactive compounds have been performed to identify molecules that modulate cellular processes relevant to cell therapy, few have been translated into promising in vivo preclinical results. One example of interest is a stabilized prostaglandin found to improve hematopoietic stem cell homeostasis using a high throughput screen in zebrafish that yielded a cord blood transplantation approach.

### Summary and Results.

As an advantageous option, we describe the manipulation of signaling pathways via small molecule pretreatment as a simple, cost-effective and scalable approach to improve control over cell fate. Furthermore, since therapeutic approaches can be designed with small molecules that are not delivered directly to patients but only used to pretreat cells prior to cell infusion, safety is yet another advantage of this approach.

Human mesenchymal stem cells, which are of interest for cell therapy, exhibit inefficient homing to sites of inflammation following systemic administration. One of the reasons is very low expression by huMSCs of potentially significant homing ligands on the cell surface, such as CD11a (LFA-1), which may mediate cell interactions with activated endothelium and participate in the homing cascade.

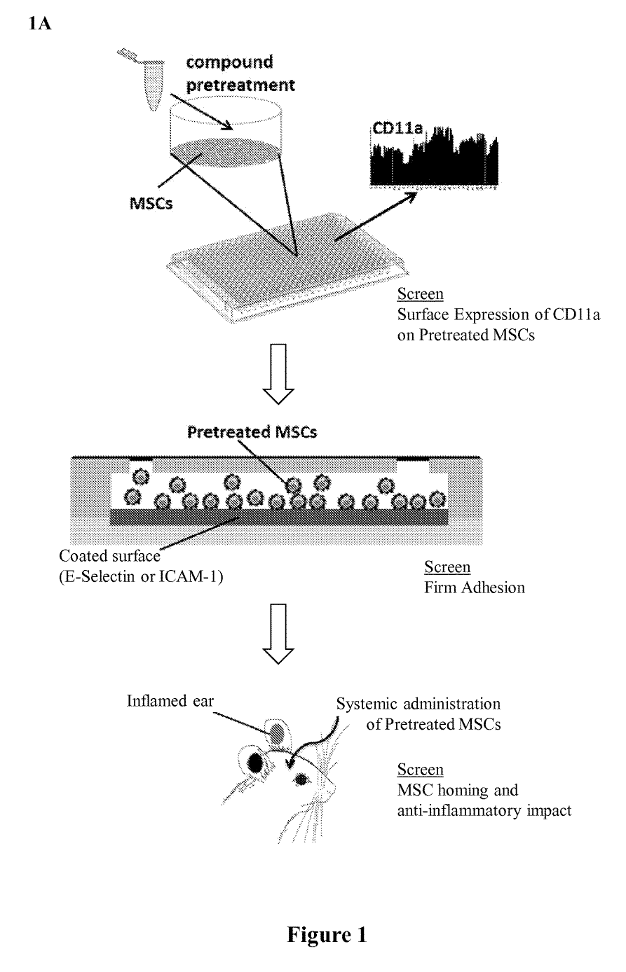

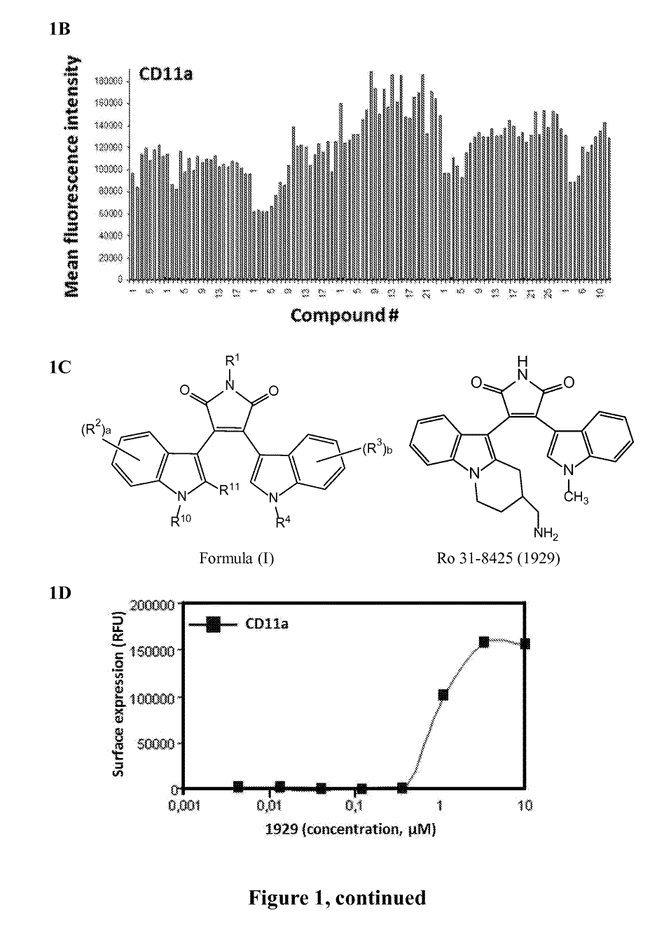

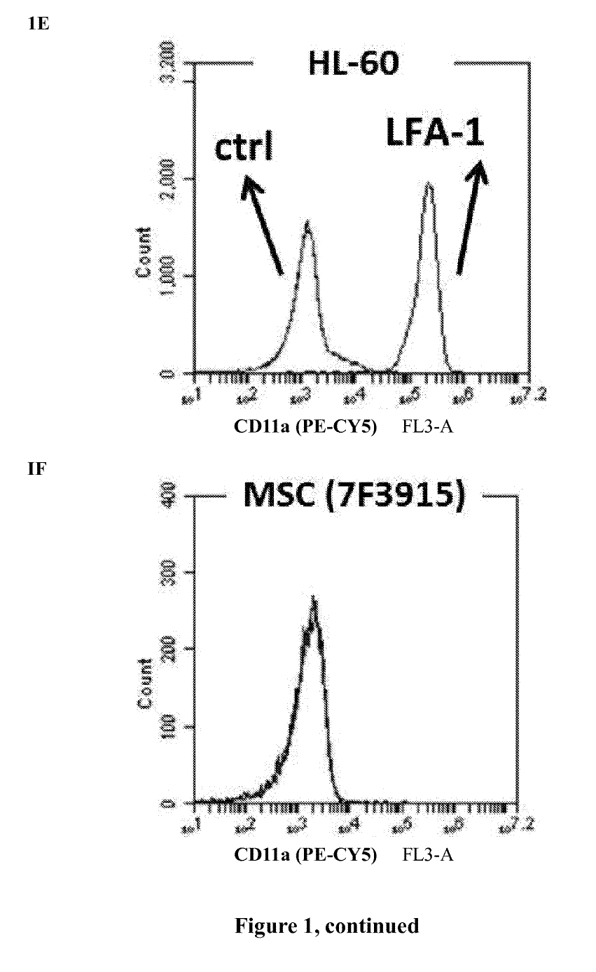

In this study, we aimed to increase MSC surface expression of key homing ligands via small molecule pretreatment to improve homing of systemically administered MSCs to sites of inflammation (FIG. 1A). Integrins, such as VCAM-1, were previously implicated in MSC homing, and engineering MSCs (via antibody coating or viral DNA transfection) to over express integrins can promote targeting of systemically infused MSCs to disease sites. We focused on surface expression of CD11a, otherwise known as integrin alpha L. CD11a combines with integrin beta 2 (CD18) to create lymphocyte function-associated antigen-1 (LFA-1), which serves a central role in mediating leukocyte firm adhesion, an important step in the inflammatory leukocyte homing cascade. Lack of CD11a expression on the surface of culture-expanded MSC and high expression levels on promyelocytic leukemia cells (HL-60, positive control) were confirmed via flow cytometry (FIG. 1E, 1F).

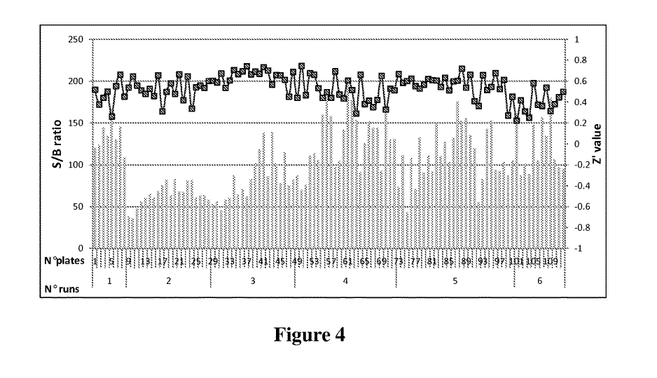

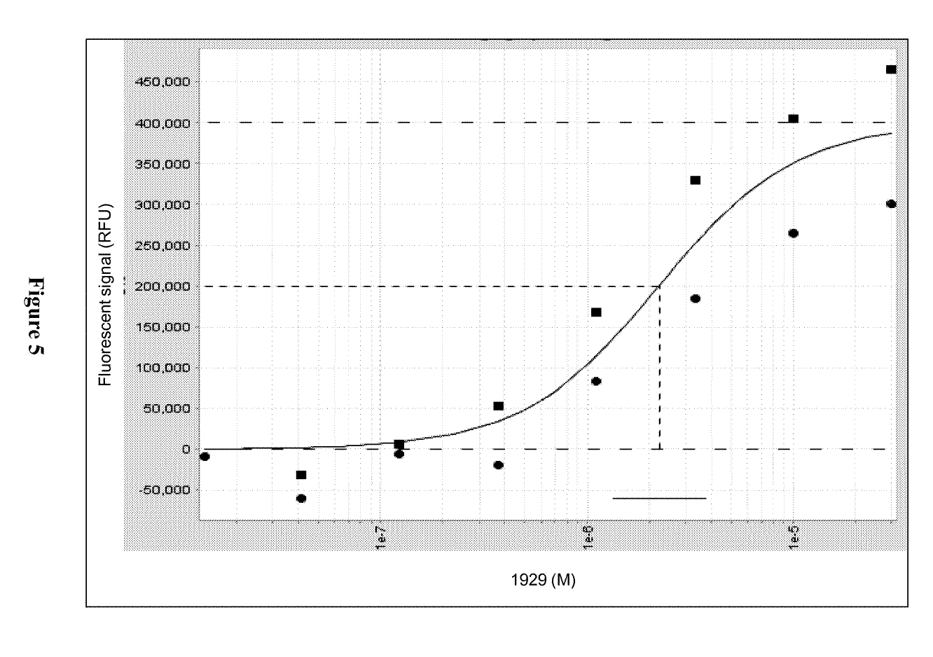

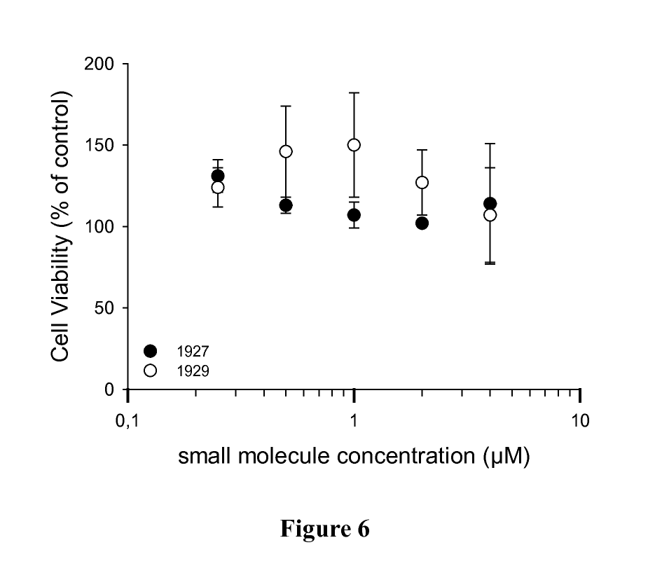

For maximal detection of CD11a on cell surface, multiple fluorophore-conjugated anti-CD11a antibodies were tested, and a PE-CY5-conjugated anti-CD11a was validated (clone:HI111). This anti-CD11a antibody and a corresponding isotype control were used in a medium throughput screening of 9,000 compounds in a small molecule library including a collection of 2,500 cellular signaling pathway activators and inhibitors (named by acronym SPAI) to identify candidate molecules that increase expression of CD11a on the MSC surface, relative to untreated or negative control cells. Cells were pretreated with a low (0.104) or high (3 μM) concentration of small molecules for 24 h, followed by incubation for one hour with a PE-CY5-conjugated anti-CD11a antibody to detect its expression on MSC surface. Surface expression levels of ligands were then evaluated by the Acumen laser-scanning fluorescence microplate cytometer. Certain small molecule compounds were found to significantly increase surface expression of CD11a (FIG. 1B; FIG. 4). In FIG. 4, the data show screening results relating to surface marker signal detection for pretreated MSCs, and the assay parameter of the Z-factor (Z′ value) demonstrates that these screening assays are robust. Cell viability in response to compounds was also tested; the MSCs remained viable, confirming that the molecules are not toxic at these concentrations after 24 hours of pretreatment (FIG. 6). From this screen, we identified a small molecule which is a selective PKC inhibitor as capable of increasing surface expression of CD11a. FIG. 1C shows the structure of this small molecule, Ro 31-8425 (also designated compound 1929, and CAS#131848-97-0); see also Muid R. E. et al., FEBS Letters 1991, 293:169-172. This compound induced a two-fold higher CD11a surface expression compared to HL-60 cells. Interestingly, this compound had no observed effect on the expression of other homing ligands. Pretreatment of MSCs with compound 1929 induced a significant increase in CD11a surface expression (FIG. 1D and FIG. 5, indicating the amount of surface expression as a function of pretreatment concentration level).

FIG. 1C also shows the structure of a generic formula (I) for embodiments of active compounds relating to the invention.

Importantly, positive hits of small molecule compounds identified to increase CD11a expression on MSCs from one donor induced a similar magnitude effect on MSCs from a different donor source. Establishing a donor-independent response is advantageous for successful clinical translation of autologous and heterologous (allogeneic) cell therapy. It is also important for allogeneic therapy given the need for additional donors in light of the possible eventual exhaustion of the master cell bank.

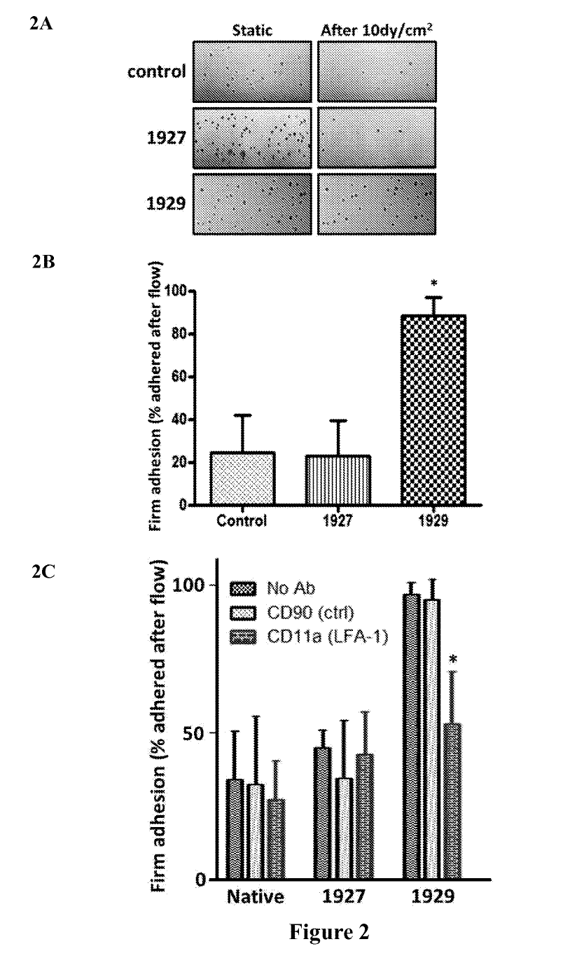

Considering the key role of CD11a in mediating leukocyte firm adhesion, we next assessed the effect of the identified CD11a-upregulating hits on MSC firm adhesion, which is governed by CD11a and is a major step in the leukocyte homing cascade. MSCs were previously shown to exhibit inefficient dynamic interactions with selectins, which may be responsible for their limited homing capability to inflamed sites. Therefore, we tested firm adhesion of pretreated MSCs to E-selectin that is up-regulated on the endothelial surface at sites of inflammation and is involved in leukocyte recruitment during inflammation. MSCs were incubated with certain compounds at a concentration of 3 μM for 24 h. Then the MSCs were subjected to a firm adhesion assay under physiologically relevant shear flow using a multiwell plate microfluidic system. MSCs (3-5×10⁶ cells/ml) were introduced into an E-selectin-coated microfluidic channel, permitted to adhere and then subjected to increasing shear flows (0.25-10 dynes/cm²). Pretreatment with selected compounds that increased CD11a expression also increased MSC firm adhesion in vitro to an E-selectin-coated substrate compared to native, vehicle-treated MSCs. Specifically, firm adhesion to E-selectin of MSC pretreated with Ro-318425 (1929) was 3-fold higher compared to control MSCs (FIG. 2A, 2B). In contrast, compound 1927, which did not increase expression of CD11a, also did not improve MSC firm adhesion to E-selectin-coated substrates (FIG. 2A, 2B).

To explore the possible involvement of CD11a in mediating pretreated MSC firm adhesion to an E-selectin-coated surface, we performed antibody blocking experiments. Following pretreatment with compound 1929 or 1927 (at a concentration of 3 μM for 24 hours), a suspension of MSCs was incubated for 30 minutes with antibodies against CD90 (as control) or CD11a followed by an E-selectin firm adhesion assay. Incubating with CD11a blocking antibody significantly reduced 1929-pretreated MSC firm adhesion to E-selectin-coated surface, (˜90% of adhered cells to 50%) following CD11a blocking (FIG. 2C). This strongly suggests that CD11a, which was upregulated in response to 1929 treatment, is directly involved in mediating the increased MSC firm adhesion to E-selectin. However, CD11a blocking did not abolish 1929-pretreated MSC firm adhesion back to control untreated MSC levels, suggesting that other surface markers are also involved in mediating the increased firm adhesion of 1929-treated MSCs to E-selectin. In contrast, antibody blocking of CD90 had no effect on firm adhesion. Antibody blocking of CD11a or CD90 had no effect on 1927-pretreated MSC firm adhesion (FIG. 2C).

Compounds that significantly increased MSC firm adhesion to E-selectin in vitro were then tested in vivo for their ability to promote targeting of systemically administered MSCs to a site of inflammation. In our murine model, one ear pinna is injected with lipopolysaccharide (LPS) to induce local inflammation, while the other receives a saline injection (control). This model provides good sensitivity in the assessment of anti-inflammatory activity. Also, good sensitivity is achieved regarding the detection of compounds inducing upregulation of a firm adhesion ligand, CD11a.

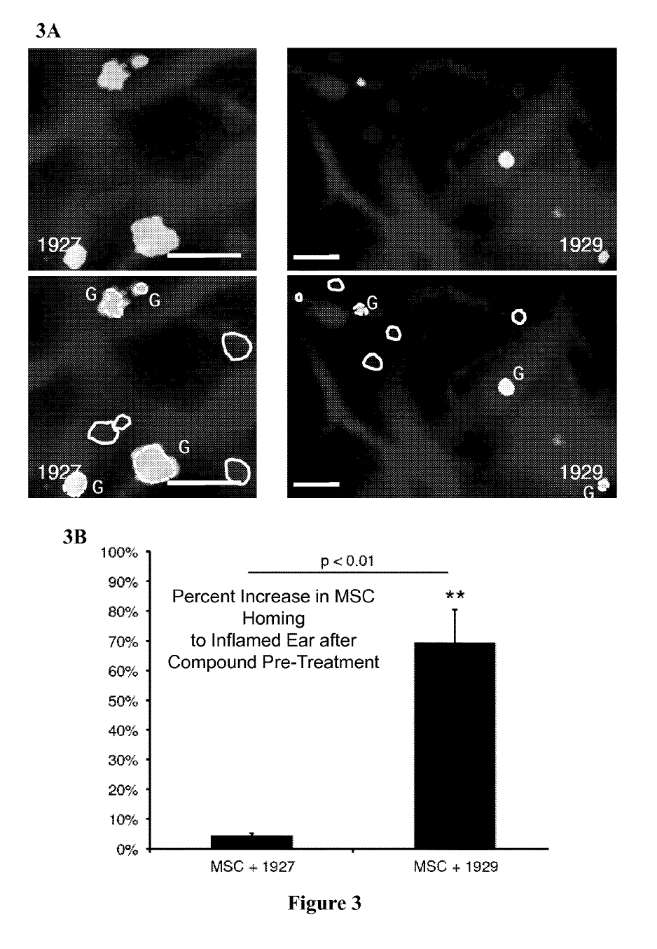

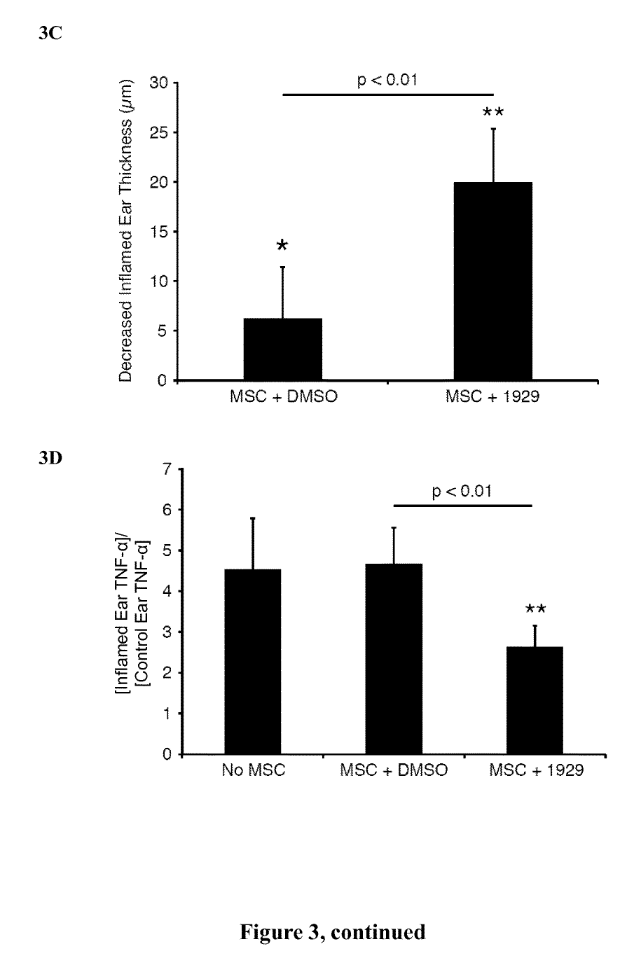

Briefly, MSCs were pretreated for 24 h with either a small molecule (3 μM in 0.1% DMSO) or a vehicle control (0.1% DMSO), stained with different membrane tracker dyes, mixed (1:1 ratio) and infused systemically (via retro-orbital injection) into mice 24 h post-LPS injection. After 24 h, cell homing to the inflamed and control ears was imaged using intravital microscopy (FIG. 3A). Pretreatment with compound 1929 significantly improved MSC homing to skin in the inflamed ear upon systemic administration, with an average of 45.2±8.6 cells/cm² for vehicle-MSCs and 78.5±15.9 cells/cm² for 1929-MSCs (69.3±11.3% increase compared to vehicle-treated MSCs, p<0.05, Tukey's HSD test). In contrast, the negative control molecule 1927 had no impact on MSC homing to the inflamed ear in vivo, with an average of 48.4±26.1 cells/cm² for vehicle-MSCs and 47.2±20.4 cells/cm² for 1927-MSCs (FIG. 3B). This data demonstrates a strong relationship between the results for the effect of a test substance on surface expression of our selected homing ligand (CD11a), E-selectin firm adhesion, and in vivo homing of systemically transplanted MSCs to sites of inflammation.

We then sought to assess the ability of 1929-pretreated MSCs, which exhibited increased homing to the inflamed ear, to alleviate the severity of LPS-induced local inflammation. To evaluate ear inflammation, ear thickness and local levels of the pro-inflammatory cytokine TNF-alpha in mice ears were measured 24 hours post administration of either vehicle or 1929-pretreated MSCs. While mice treated with control MSCs exhibited a small reduction in ear thickness (6.3±5.2 μm reduction, p<0.05 vs. no MSC control), the MSC pre-treated with compound 1929 exhibited an over 3× greater effect on ear swelling (20.0±5.3 μm reduction, p<0.01 vs. no MSC control and vehicle-treated MSCs) (FIG. 3C). LPS-induced inflammation resulted not only in ear swelling but also in a significant increase in local levels of the pro-inflammatory cytokine TNF-alpha in the inflamed ear compared to the saline-treated ear (4.5±1.3 fold TNF-alpha increase in inflamed versus the control ear). Consistent with the cell delivery and ear thickness data, the increased TNF-alpha levels in the inflamed ear was reduced by administration of 1929-treated MSCs (2.6±0.5 fold reduction, p<0.01 versus the results observed with no MSCs and vehicle-treated MSCs (FIG. 3D)). Vehicle-treated MSCs did not impact TNF-alpha levels. Taken together, these results strongly show that systemic infusion of 1929-pretreated MSCs, which display increased homing to inflamed tissues, also resulted in improved anti-inflammatory therapeutic effect.

Our multi-step screening process identified small molecules that increased expression of CD11a on the MSC surface, enhanced MSC firm adhesion to an E-selectin-coated substrate and also promoted MSC homing to sites of inflammation following systemic administration, resulting in improved anti-inflammatory response.

Our findings are supported by other approaches that enhanced MSC therapeutic impact via improved homing to disease sites. Recently, we have shown that mRNA-induced expression of SLeX/PSGL-1 (rolling ligands) resulted in a transient improvement of only 30% in MSC homing in the same local inflammation model and yielded a limited anti-inflammatory impact of PSGL-1/SLeX MSCs. In the current work, the 70% increase in MSC delivery to an inflamed site from small molecule induction of CD11a (which mediates firm adhesion) and commensurate improvement in MSC anti-inflammatory response suggest that upregulation of firm adhesion ligands is an attractive target to improve cell homing.

A significantly active small molecule identified in this example was Ro 31-8425 (coded compound 1929 in the screen), a highly selective PKC inhibitor. PKC activation can stimulate MSC retention in infarcted myocardium by activation of focal adhesion kinase following local administration, and can mediate delta-opioid receptor activation-induced MSC survival. Interestingly, PKC inhibition was previously shown to partially inhibit acetylcholine-induced MSC migration, and a role for PKC in non-canonical Wnt-mediated MSC osteogenic differentiation was also proposed. However, to our knowledge this is the first report demonstrating that PKC inhibition promotes CD11a expression and MSC firm adhesion as well as systemic targeting of MSC to an inflamed site.

Our demonstration of correlating cell surface adhesion receptor expression to in vitro and in vivo adhesion, and further to therapeutic response, provides the foundation for exogenous cell therapy where targeting of cells to diseased or damaged tissues is important.

Our approach of small molecule screening further extends to using a cocktail of small molecules to target complementary pathways and simultaneously improve cell homing and control of the secretome, thus generating an exogenous population of cells with improved therapeutic properties. Furthermore, small molecule pre-conditioning to enhance homing can be combined with other bioengineering strategies to facilitate targeted delivery of therapeutics to disease sites. We can take advantage of the knowledge base regarding endothelial receptor expression on vessels in specific tissues which is well characterized, providing zip codes that can help identify hits of homing-inducing compounds to achieve delivery of cells to specific tissues (e.g. MADCAM-1 is expressed in gut endothelium, PNAd in peripheral lymph nodes). Hence, small molecule pretreatment can serve as an effective methodology to target cells to a selected tissue.

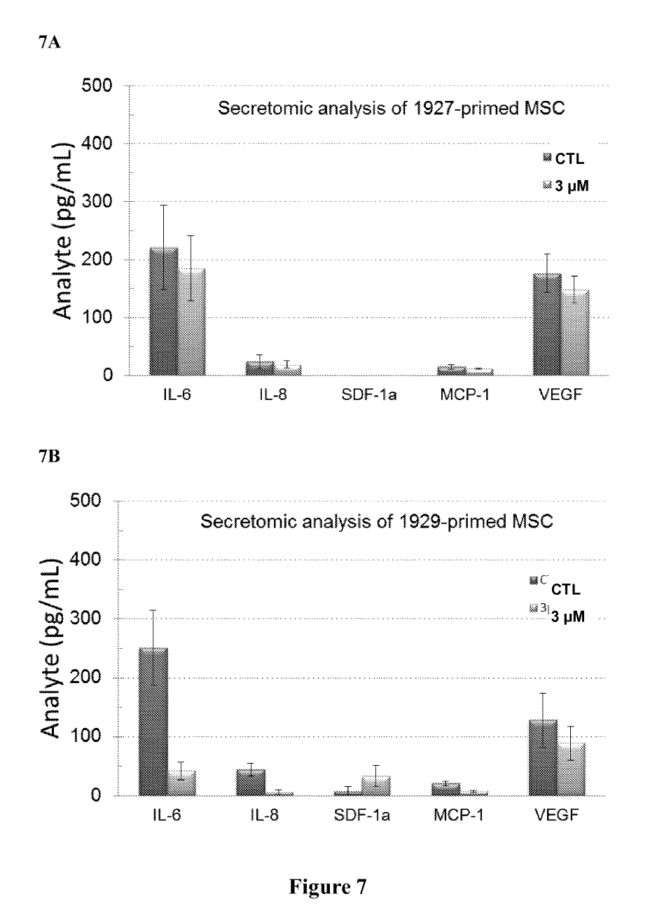

The impact of the ability to induce MSC secretome phenotype was characterized. We conducted tests to assess the impact of pretreatment of MSC with compounds 1927 and 1929 on the profile of various secretome components produced. Results are shown in FIG. 7. In FIG. 7A, results indicated no secretome changes upon pretreatment of MSC with compound 1927 (3 μM for 24 hours) of MSC in vitro. In contrast, the response to pretreatment of MSC with compound 1929 demonstrated the downregulation of IL-6, IL-8 and MCP-1 levels and upregulation of the SDF-1alpha level (FIG. 7B). MSC pretreatment conditions in each case were at compound concentrations of 3 micromolar for 24 hours in vitro.

In conclusion, we have described a multi-step screening process that identifies small molecule compounds for improving homing particularly for mesenchymal stem cells. The screening has also been used to discover an important family of compounds. In addition to the specific compound 1929, a large family of compounds is provided as exemplified elsewhere herein which is useful in improving homing and therapeutic applications of MSCs.

### Methods

Mesenchymal Stromal Cell Culture and Compound Pretreatment.

Bone marrow-derived MSCs were purchased from Lonza (Walkersville, Md., USA; catalog number: PT-2501, donor #7F3915) and expanded in MSCGM Mesenchymal Stem Cell Growth Medium (Lonza, catalog # PT-3001). Cells were kept at 37° C. with 5% CO2 and media was changed every 3 days. Cells were passaged using 1% trypsin-EDTA solution. MSCs at passage 3-6 were used for all experiments. For compound pretreatment, pre-confluent MSC were incubated for 24 hours with the indicated compounds (3 μM).

Human Promyelocytic Leukemia Cells Cell Culture and Compound Treatment.

The HL-60 cells were from the American Type Culture Collection (ATCC). Cells were seeded in Iscove's Modified Dulbecco's Medium-GlutaMax containing 20% FBS (LifeTechnologies).

Medium Throughput Screen.

An in vitro screen was performed for compounds that induce surface expression on huMSCs of the marker CD11a (LFA-1). This screen may be considered a medium throughput screen. Certain compounds that induce the increase of this surface marker were then selected for testing in an in vitro firm adhesion assay and/or in vivo systemic administration in an animal model of inflammation. The surface expression screen involved fluorophore-conjugated antibodies to the respective markers. Following pretreatment with the test compounds, samples of cells were incubated with the labeled antibodies for detection of surface marker expression and change relative to a reference level of untreated cells or negative control samples or values. Cells were pretreated with a concentration (3 μM) of each small molecule compound for 24 hours, followed by incubation with a fluorophore-conjugated anti-marker antibody to detect expression on MSC surface. Surface expression levels of marker ligands were then evaluated by laser-scanning fluorescence microplate cytometer. Certain test compounds were found to significantly increase surface expression of one or more markers. Cell viability in response to compounds was also tested to assess whether molecules have toxicity after 24 hours of pretreatment. Testing was performed on two different huMSC sources representing cells from different donors. A high level of consistency was observed in the response of the cells, regardless of donor source, to the effect of the test compounds on induction of cell surface marker expression.

Further details of the screening approach are provided as follows. A unique single batch of bone marrow-derived MSC cells was prepared for the entire screening campaign by cell amplification using CellStack flasks and freezing in separate vials at passage P5. Flow cytometry experiments demonstrated the expected immunophenotype profiles for MSC upon 24 hours after thawing. For each run, vials were thawed and cells directly seeded on 384-well plates at 3,000 cells per well in MSCGM medium. Following an overnight incubation, cells were treated with the indicated concentrations of compounds for 24 hours. Cells were washed in PBS, 3% BSA and incubated for one hour with PE-CY5-conjugated anti-CD11a monoclonal antibody (clone HI111). Expression of CD11a at the cell surface was detected using the Acumen Explorer, a laser-scanning fluorescence microplate cytometer after two PBS washings. HL-60 cells seeded at 10,000 cells per well and treated with 100 nM phorbol 12-myristate 13-acetate (PMA) for 24 h to induce cell adhesion were used as a positive control in each 384-well plate. Positive compounds were counter-screened for their auto-fluorescence by measuring the signal in the absence of antibody.

Exemplary Screen.

A single batch of bone marrow-derived MSC was prepared for the entire screening campaign by cell amplification using CellStack flasks and freezing in separate vials at passage P5. Flow cytometry experiments demonstrated the expected immunophenotype profiles 24 h after thawing. For each run, vials were thawed and cells directly seeded on 384-well plates at 3,000 cells per well in MSCGM medium. Following an overnight incubation, cells were pretreated with a low (0.1 μM) and high (3 μM) concentration of the compounds for 24 h. 9,000 cpds were tested in 112 assay plates, each 384-well plate contained 320 cpds (from column 5 to 24), one column for Max (Column2, HL60 cells) and one column for Min (Column4, MSC cells). Cells were washed in PBS, 3% BSA and incubated for one hour with PE-CY5-conjugated anti-CD11a monoclonal Ab (BD Biosciences). Expression of CD11a at the cell surface was detected after two PBS washes using the Acumen Explorer equipment, a laser-scanning fluorescence microplate cytometer. HL-60 cells (seeded at 10,000 cells per well and treated with 100 nM phorbol 12-myristate 13-acetate (PMA) for 24 h to induce cell adhesion) were used as a positive control in each 384-well plate. Positive compounds were counter-screened for their auto-fluorescence by measuring the signal in the absence of Ab. Shown in FIG. 4 is the global screening data. Using the Max, Min columns, we have calculated Max/Min ratio generated with CD11a Ab for each assay plate (signal/background ration in green columns was calculated for each of the 112 assay plates). The Z-factor=1-((3×Standard dev Max+3×Standard dev Min)/(Mean Max Mean Min), with Z-factor (top curve) is determined for each assay plate based on Max, Min data and the standard deviation obtained (n=16 wells of Max and 16 wells of Min). For a successful cell-based assay, the Z-factor should be between 0.5 and 1.0.

Cell Viability Assay.

Cell viability experiments were performed using the IncuCyte FLR (Essen Bioscience). Cells were stained with a cell impermeant cyanine dimer nucleic acid stain (Yoyo-1, Life Technologies). This fluorescent dye was used to measure cell membrane integrity. Pre-confluent human mesenchymal stromal cells (MSCs) were incubated for 24 hours with both the indicated small molecules (0.25 to 404) and Yoyo-1 dye (0.404). To kinetically measure MSC membrane integrity, images were acquired every two hours using an IncuCyte FLR live-cell image imaging system.

E-Selectin and ICAM-1 Firm Adhesion Assays.

Cell adhesion experiments were performed using Bioflux1000 (FluxionBio), allowing accurate control over shear flow. A special 48-well plate was used, in which a microfluidic channel (350 μm×70 μm) connects each pair of adjacent wells (termed inlet and outlet wells). The plate was placed under vacuum and the channels were coated from the inlet with recombinant human E-selectin (5 μg/mL) or ICAM-1 (5 μg/mL) and incubated at 37° C. for 1 h. Prior to introducing the cells into the channel, a wash with PBS−/− from the outlet well was performed for 5 min. Compound-pretreated MSCs were introduced into the channel, followed by attachment period of 2 minutes (no flow applied during the attachment period). Attached cells were then subjected to increasing shear flow, ranging from 0.25 dynes/cm² for up to 10 dynes/cm². Images were acquired using the Montage software and cell adhesion to the coated channels following subjection to shear flow was examined. See also disclosure elsewhere herein.

Antibody Blocking Experiments.

Pre-confluent MSCs were incubated for 24 hours with the indicated small molecules (3 μM). MSCs were than detached, washed and incubated for 30 minutes with an antibody reagent. The following antibodies are used: anti-human CD11a (clone: HI111), anti-human CD90 (clone: MAB2067), mouse anti-human CD11a (clone: TS1/22), and mouse IgG1 isotype control. Cells were then introduced into the channel and given 2 minutes to adhere before being subjected to increasing shear flow for a firm adhesion assay on a microfluidic channel coated with E-selectin or ICAM-1.

Cell Staining.

For tracking MSCs, cells were stained with a range of lipophilic membrane dyes with emission wavelengths in the green (DiO), red (Dil), far red (DiD), and near-IR (DiR, extinction coefficient=270,000 cm⁻¹ M⁻¹ in MeOH) (Vybrant dyes, Invitrogen, Carlsbad, Calif.). Primary human MSCs were suspended at a concentration of 10e6 cells/mL and incubated with Vybrant dye (10 μM DiO, 10 μM Dil, 10 μM DiD, or 15 μM DiR) in 1×PBS+0.1% BSA for 20 minutes at 37° C. The MSCs were then washed twice in 1×PBS and mixed in equal numbers for imaging in vitro or in vivo at a concentration of 10×10e6 cells/mL for imaging.

In Vivo MSC Homing.

C57BL/6 mice (Charles River Laboratories, Wilmington, Mass.) were anesthetized with ketamine/xylazine and their ears shaved 24 h prior to cell infusion. To induce an inflammatory response, 30 μg of E. coli lipopolysaccharide (LPS, Sigma, St. Louis, Mo.) in 50 μL saline was injected into the pinna of the left ear, with 50 μL 0.9% saline injected into the right ear as a control. For in vivo dye sensitivity validation, 1×10e6 cells of each stain were suspended in 150 μL PBS (pH 7.4) and injected by retro-orbital vein infusion.

To evaluate the minimum number of cells needed for a reproducible response, each mouse (n=4) received a range of cell doses (10,000, 50,000, 100,000, or 500,000) each of cells stained with Dil or DiD with a dye switch (i.e., dyes used for experimental and control cells were alternated to prevent bias based on the dyes that were used). To evaluate the impact of small molecule hit pre-treatment on MSC homing to the inflamed ear, MSCs were incubated with 3 μM of the small molecule hit or 0.1% DMSO as a control for 24 h before staining and in vivo administration. To highlight the vasculature, 50 μL of 10 mg/mL FITC-dextran (2×10e6 kDa; Sigma, St. Louis, Mo.) was injected retro-orbitally prior to imaging.

Confocal Fluorescence Microscopy.

In vitro staining and in vivo homing of stained MSCs to the skin was imaged noninvasively in real time using a custom-built video-rate laser-scanning confocal microscope designed specifically for live animal imaging. For in vivo imaging, the mouse ear was positioned under a coverslip with methylcellulose gel and images acquired at 30 frames per second at a depth up to 200 μm using a 60×1.0NA water immersion objective lens (Olympus, Center Valley, Pa.). DiO labeled MSCs were excited with a 491 nm continuous wave (CW) laser (Cobalt, Stockholm, Sweden), and detected through a 520±20 nm bandpass filter (Semrock, Inc., Rochester, N.Y.). Dil labeled MSCs were excited with a 561 nm CW laser (Coherent, Inc., Santa Clara, Calif.) and detected through a 593 nm±40 nm filter (Omega Optical, Brattleboro, Vt.). DiD labeled MSCs were excited with a 635 nm CW laser (Coherent, Inc., Santa Clara, Calif.) and detected through a 695 nm±27.5 nm band pass filter (Omega Optical, Brattleboro, Vt.). DiR labeled MSCs were excited using a femtosecond Ti:Sapphire Maitai source for single photon excitation at 750 nm (Spectra Physics, Santa Clara, Calif.) and collected through a 785 nm±31 nm band pass filter (Omega Optical, Brattleboro, Vt.).

For quantification, the average number of cells in 20 representative imaging locations across the inflamed region was counted in each mouse. Cells were defined as having a diameter from 10-30 μm to eliminate debris and clumps from analysis, and a primary channel intensity greater than 2 to eliminate autofluorescent events. The average relative detection sensitivity (ratio of Dil/DiD counts) determined in the overall dye efficiency experiments were used to scale the counts for DiD stained cells. A dye switch allowed direct comparison of cell homing numbers and ensured that equalization did not introduce bias into our measurements. For multiple comparisons, Tukey's HSD test was used. Error bars in graphs represent standard error, and statistical significance is denoted by * p<0.05.

Ear Thickness and TNF-α ELISA.

To determine the impact of small molecule pretreatment on MSC therapeutic potential, ear swelling was measured. As a baseline, we measured ear thickness of all mice to be used using a caliper (Mitutoyo Inc.) and found no difference. Each measurement was taken 3 times with the average value recorded, and care taken to ensure minimal compression. Inflammation was then induced by injection of 30 μg LPS in 30 μL 0.9% saline solution into the pinna of the left ear. 24 hours later, n=3 mice of each condition were infused with no MSCs, 10e6/20 g body weight MSCs pretreated for 24 hours with 0.1% DMSO, or 10e6/20 g body weight MSCs pretreated for 24 hours with 3.0 μM small molecule compound 1929. Upon 24 hours after cell infusion, ear thickness was measured using a caliper as before. To evaluate TNF-α secretion, inflammation and MSC treatment was performed as above with n=4-6 mice for each condition. Mice were sacrificed 24 hours after cell administration and both ears were harvested. Ears were then ground in ice-cold extraction buffer (RIPA with 0.5% Tween-20) using a homogenizer. Homogenates were transferred to 1.5 mL tubes, centrifuged at 13,000×g for 10 minutes at 4° C., and the supernatant was stored at −80° C. until analysis. The level of mouse TNF-α level in the samples was quantified using an anti-mouse TNF-α ELISA kit (Biolegend, San Diego, Calif.).

Administration.

In vivo systemic administration as noted herein was performed by intravenous infusion using injection via the retro-orbital venous sinus. In other embodiments, systemic administration can be performed with vascular delivery via venous or arterial systems. More specifically, delivery can be performed via coronary, jugular, hepatic, femoral, and other routes as understood by a person of ordinary skill in the art. In certain embodiments, delivery of MSCs can occur by local administration. As an example, pretreated MSCs can be locally injected at an inflammation site directly into a tissue or to a nearby circulatory or lymphatic vessel.

Cell Compositions, Pharmaceutical Compositions, and Pharmaceutical Carriers.

In embodiments, compositions of cells are provided. In an embodiment, the cells are prepared as a pharmaceutical composition with a pharmaceutically acceptable carrier. In embodiments, the carrier is pharmaceutically acceptable relative to a patient, and the carrier is acceptable with respect to viability and/or function, including homing function, of the cells for administration as a pharmaceutical agent to the patient. In an embodiment, the pharmaceutical carrier comprises a physiologically compatible aqueous solution for suspension or reconstitution of the MSCs. In embodiments, the solution is buffered. In embodiments, the solution is supplemented with nutrients and/or components as understood in the art.

Secretomic Analysis of Small Molecule-Primed MSCs.

A single batch of human MSCs (#7F3915) was used for all of these experiments. Cells were seeded at 25,000 cells/well in a 12-well microplate. The following day, cells were treated with the selected compound (3 μM) or DMSO (as control). After 24 hours of treatment, secretomic samples were collected, centrifuged and frozen. Secretomes of MSCs were assayed for cytokines, chemokines and growth factors using Bio-plex human 21-plex and 27-plex immunoassay kits (Bio-Rad), according to the manufacturer's instructions. The 27-plex and 21-plex panels consisted of the following analytes: IL-1alpha, IL-1beta, IL-1Ralpha, IL-2, IL-3, IL-4, IL-5, IL-6, IL-7, IL-8, IL-9, IL-10, IL12p40, IL12p70, IL-13, IL-15, IL-17, IL-18, CTACK, GROalpha, HGF, IFN-alpha2, LIF, MCP-1, MCP-3, MIF, MIG, beta-NGF, SCF, SCGF-beta, SDF-1alpha, TNF-alpha, TNF-beta, TRAIL, Eotaxin, FGF-2, G-CSF, GM-CSF, IFN-gamma, IP-10, MIP-1alpha, PDGF-bb, RANTES and VEGF. A standard range of 0.2 to 3,200 pg/mL was used. Samples and controls were run in triplicate, standards and blanks in duplicate. Error bars represent standard deviation (3 independent experiments).

Cell Viability Assay.

Pre-confluent MSCs were incubated with Ro-31-8425 at the indicated concentrations for 24 h or 72 h and cell viability was assessed via an XTT assay according to manufacturer's instructions (ATCC).

### Example 2: Cell Adhesion Assay

Methods for Cell Rolling/Firm Adhesion Protocols.

These protocols use the BioFlux 1000 system. The following are general comments. Rolling and firm adhesion assays can be performed on protein coating or cell monolayer created inside the microfluidic channels. The first step of every experiment must include priming of the microfluidic channel by introducing PBS/media into the channel to prevent air bubbles. This is easily done by adding 100 μl of the desired liquid into the wells and applying shear force of 2-5 dynes/cm² to introduce the liquid into the channel.

Protein Coating.

1. Prepare protein solution in the desired concentration. a. For different Selectins (P-selectin, E-selectin, L-selectin) use 2.5-10 μg/ml. b. For Fibronectin (to be used for cell seeding) use 20-30 μg/ml. c. For Gelatin (to be used for cell seeding) use 0.1% (v/v). 2. Add 50-100 μl of protein solution to outlet. Apply shear force of 2 dynes/cm² for 5 minutes to coat the channel. Incubate to allow adsorption to bottom: Selectins: 1 hr at 37 degrees C., Fibronectin: 15-30 min at room temperature. 3. Aspirate liquid from wells. Wash channel with PBS (2 dynes/cm² for 5 min). Channel is now coated and ready to be used.

Creation of Cell Monolayer.

1. Coat channel with appropriate protein coating. For MSC (human bone marrow mesenchymal stem cells), coating for cell seeding uses Fibronectin (20 μg/ml, 15-30 min at R.T). 2. Trypsinize cells, wash cells once with appropriate full media. 3. Using the suitable cell concentration is crucial for obtaining a confluent channel: For MSC: use 5 million cells/ml. 4. Add 50 μl of cell suspension in the appropriate concentration to the inlet. 5. Introduce cells into the channel (2 dynes/cm² until cells fill the channels). 6. Fill both outlet and inlet with 200 μl. Let the cells sit and adhere for 6-8 hr in the incubator (37° C., 5% CO₂). 7. Following 6-8 hr, wash the channel with full media (2 dynes/cm² for 10-15 min) to remove unattached cells. 8. For CHO: Channel is ready for use at the same day. For MSC: Let the cells sit in the channel overnight in an incubator (both wells must be filled with equal volume of media). In the morning wash again, and then channel is ready for use.

Cell Rolling.

1. Coat the microfluidic channel with the desired protein/cell monolayer. 2. Prepare cell suspension to be rolled: For MSC use 3 million cells/ml. 3. Add 50 μl of cell suspension to outlet well. 4. Introduce cells into channel by applying shear force of 2 dynes/cm² (cells should be observed within 10-15 sec flowing from outlet to inlet). 5. To record rolling in different shears, reduce shear to 0.25-0.5 dynes/cm² and acquire 20-30 sec videos (using stream acquisition of the MONTAGE software) in each desired shear (better to increase shear gradually from 0.25 up to 2 dynes/cm². Higher shears are also possible). 6. Cells paths and rolling velocities can be analyzed by the MONTAGE software.

Firm Adhesion.

1. Obtain preferred channel coating (protein/cell monolayer). 2. Prepare cell suspension (same concentrations detailed above for different cell types). 3. Add 50 μl of cell suspension to outlet well. 4. Introduce cells into channel by applying shear force of 2 dynes/cm² (cells should be observed within 10-15 sec flowing from outlet to inlet). 5. Once cells are observed in the channel, stop the flow and allow the introduced cells to sit for different time periods ranging from 30 seconds to 5 minutes. 6. Following this settling period, apply shear force starting from 0.5 dynes/cm² up to 10 dynes/cm² and acquire videos using stream acquisition. 7. Cell quantification is then performed using the Montage software—percentage of cells adhered, rolled or washed away after applying shear flow should be quantified.

Inflammation Model.

1. Coat channel with Fibronectin as described above 2.

Create a monolayer of HUVEC/LMVEC as described above. 3. After the cells spread overnight, prepare a 10 ng/ml solution of TNF-α in basal endothelial media (5-50 ng/ml was examined, 10 ng/ml was shown to be the optimal concentration to induce endothelial activation). 4. Add 100 μl of TNF solution to outlet. 5. Introduce TNF into the channel by applying shear flow (2 dynes/cm² for 5 min). 6. Incubate channel with TNF for 6 h (37° C., 5% CO₂). 7. Wash channel with basal media. 8. Endothelial cells in the channel are now activated, simulating an inflammatory condition. 9. Channel can be used for cell rolling or firm adhesion assay.

### Example 3: Compounds and Results of Screening Assays

Results of screening assays (in vitro cell surface expression of homing marker ligands, in vitro firm cell adhesion, and in vivo systemic administration in mammalian inflammation assay) are shown in tables below. The results are of homing functions upon pretreatment of huMSCs. Results shown here may duplicate certain results described elsewhere herein. A percent increase value is considered relative to result from untreated and/or negative control MSCs. In Table 1, the identity of certain small molecule compounds and the ability to induce upregulated expression of a cell surface ligand marker are set forth. In Table 2, the molar concentrations of certain compounds inducing expression of a cell surface marker are indicated.

TABLE 1
Small molecule compound induction of homing function -
expression of cell surface ligand CD11a.

| Compound | Structure, | Cell surface ligand* |
|---|---|---|
| Code | Formula | CD11a |
| 1927 | II | NA |
| 1921 | I-4 | A |
| 1919 | I-1 | A |
| 1933 | I-2 | A |
| 1934 | I-9 | A |
| 1912 | I-10 | A |
| 1929 | I-1 | A |
| 1928 | I-14 | A |
| Ruboxistaurin | III | NA |

*NA = negative activity result, A = positive activity result

TABLE 2
Results of compound pretreatment and cell surface expression.

| | | Cell Surface Marker ED 50 (M) |
|---|---|---|
| Compound | Structure, | Molar Concentration of Compound |
| Code | Formula | for CD11a Expression |
| 1933 | I-2 | 2.49E−05 |
| 1921 | I-4 | 1.75E−05 |
| 1905 | I-5 | 1.68E−05 |
| 1906 | I-6 | 3.19E−06 |
| 1919 | I-1 | 2.75E−06 |
| 1929 | I-1 | 0.8E−06 |
| Ruboxistaurin | III | >3E−05 |
| 1927 | II | >3E−05 |

Listed below are several compounds that are capable of improving the homing capacity of human mesenchymal stem cells according to results from in vitro functional firm adhesion assays.

TABLE 3
Small molecule compound induction of homing
function - firm adhesion assay of cells.

| Compound | Structure, | Result, in vitro | |
|---|---|---|---|
| Code | Formula | Firm Adhesion* | Percent increase |
| 1919 | I-1 | A | 81% |
| 1929 | I-1 | A | 88% |
| 1933 | I-2 | A | 75% |
| 1927 | II | NA | 22% |
| Ruboxistaurin | III | NA | 0% (negative response) |

*NA = negative activity result, A = positive activity result

In vivo functional results. Of certain positive compounds identified in vitro, several tested by administering pre-treated huMSCs to the inflamed ear demonstrated a significant increase in homing activity (p<0.05, unpaired Students t-test). The increase is determined relative to results from untreated cells.

TABLE 4
Small molecule compound induction of homing function -
in vivo activity in systemic administration.

| Compound | Structure, | In vivo functional | Increase of MSC homing |
|---|---|---|---|
| Code | Formula | assay (homing)* | to inflamed ear (%) |
| 1919 | I-1 | A | 45% |
| 1933 | I-2 | NA | <10% |
| 1929 | I-1 | A | 73% |
| 1927 | II | NA | 0% |

*NA = negative activity result, A = positive activity result

Compound 1927 serves as a negative control compound in pretreatment of huMSCs for various assays. The structure of compound 1927 is a compound having formula II.

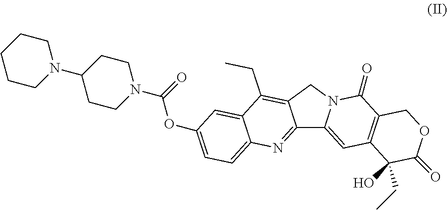 [SMILES: CCc1c2c(nc3ccc(OC(=O)N4CCC(N5CCCCC5)CC4)cc13)-c1cc3c(c(=O)n1C2)COC(=O)[C@]3(O)CC]

The compound ruboxistaurin can also serve as a negative control compound. The structure of ruboxistaurin is shown below as a compound having formula III.

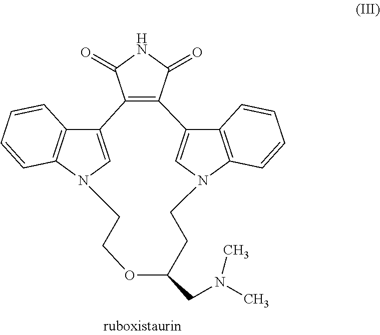 [SMILES: CN(C)C[C@@H]1CCn2cc(c3ccccc32)C2=C(C(=O)NC2=O)c2cn(c3ccccc23)CCO1]

### Example 4: Compound Pretreatment of MSCs and Results

Assessment was conducted for MSCs pretreated with compound.

Pretreatment with Compound Ro-31-8425 Induced CD11a Expression on the Surface of MSC.

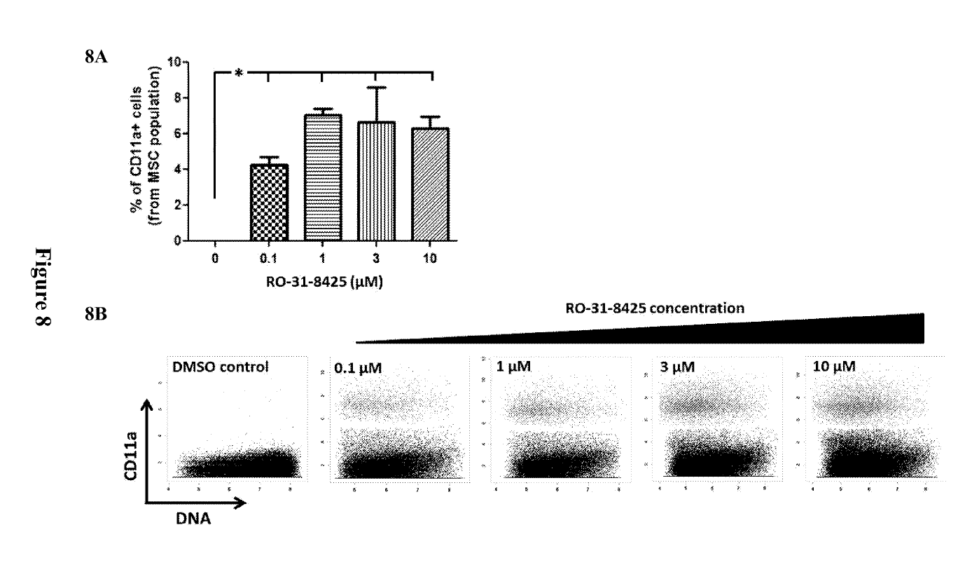

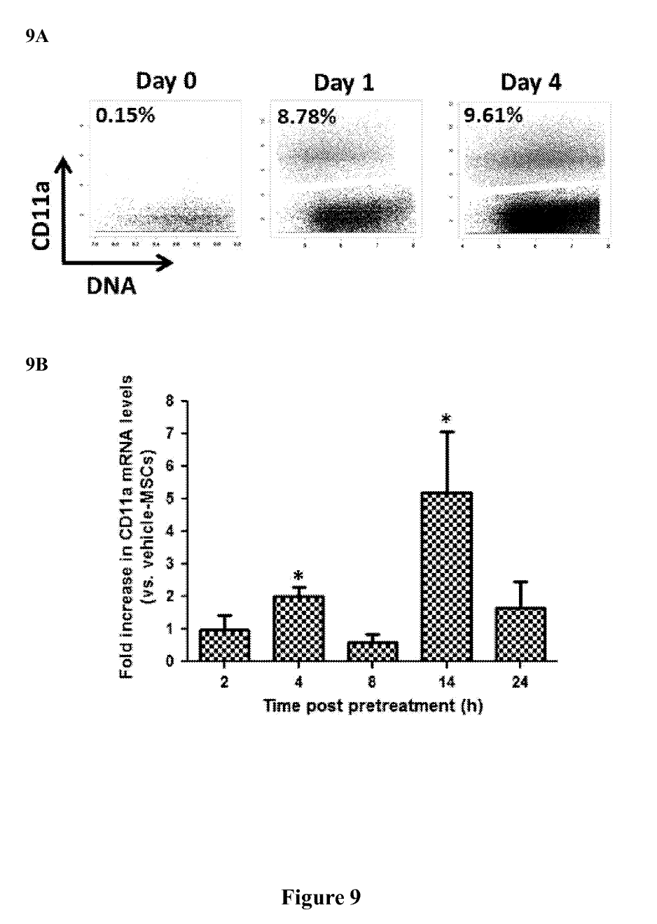

Cells were pretreated with either DMSO (0.1%) or compound Ro-31-8425 (3 μM for 24 h), followed by incubation with an anti-CD11a Ab to detect its expression on the MSC surface measured by mass cytometry (CyTOF), also referred to as cytometry by time-of-flight. As shown in FIG. 8, Ro-31-8425 induced a dose-dependent increase in the percentage of CD11a-positive MSCs. CyTOF analysis demonstrated that Ro-31-8425 treatment at 3 μM triggered a significant increase in the percentage of MSCs exhibiting surface expression of CD11a compared to virtually no CD11a+ MSCs under control conditions. The percentage of CD11a-positive MSCs in response to Ro-31-8425 (3 μM for 24 h) was stable for at least 4 days (FIG. 9A, similar pretreatment conditions were used for all subsequent experiments). As shown in FIG. 9B, RT-PCR analysis revealed that Ro-31-8425 also significantly increased CD11a mRNA levels in MSCs, with peak levels observed 14 h post incubation, indicating an impact of Ro-31-8425 pretreatment on MSC CD11a also at the transcriptional level.

The Effect of Compound Pretreatment on MSCs is Donor-Independent.

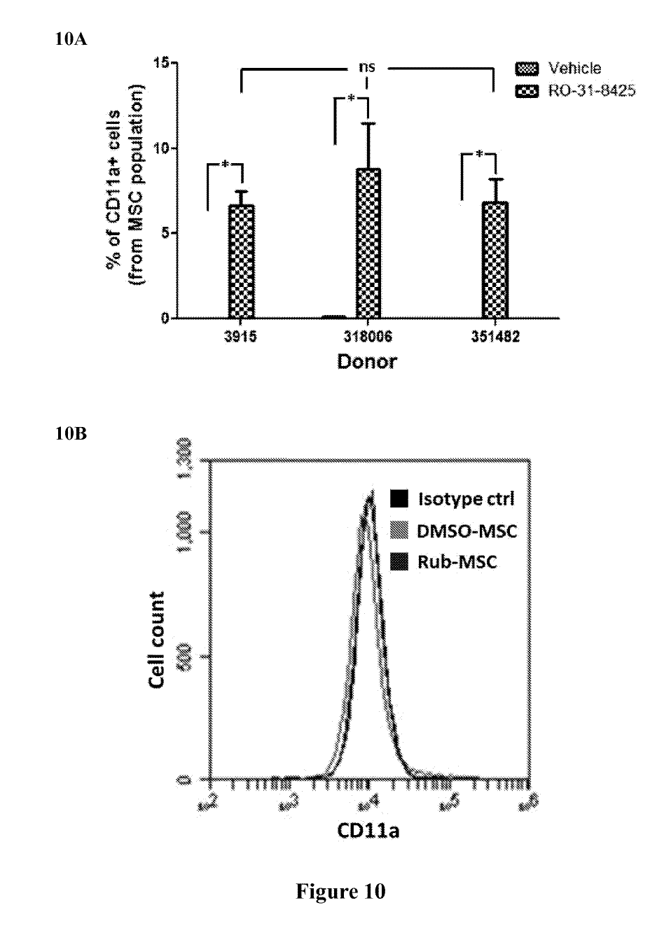

We measured the levels of CD11a surface expression on MSCs from multiple different donors in response to Ro-31-8425 pretreatment. MSCs were pretreated with DMSO vehicle control (0.1%) or Ro-31-8425 (3 μM) for 24 h, and CD expression levels were assessed via CyTOF analysis. Importantly, Ro-31-8425 increased the frequency of CD11a expression to a similar magnitude on MSCs from multiple donors (FIG. 10). Establishing a donor-independent response provides an advantage for successful clinical translation of exogenous cell therapy.

FIG. 8 illustrates the induction of expression of CD11a on the MSC surface. Pretreatment with Ro-31-8425 induced CD11a expression. FIG. 8A shows a dose-dependent increase in the percentage of CD11a+ MSCs in response to Ro-31-8425 pretreatment. MSCs were pretreated with DMSO vehicle control (0.1%) or Ro-31-8425 (0.1, 1, 3 and 10 μM) for 24 hours. The CD11a expression levels were assessed by CyTOF analysis (uppers section of dots indicate CD11a+ MSCs; lower section of dots indicate CD11a-MSCs; *=p<0.05 vs. DMSO-treated control MSCs, Tukey's HSD test).

FIG. 9B shows CD11a mRNA levels in response to Ro-31-8425 pretreatment as analyzed by RT-PCR. MSCs were pretreated with Ro-31-8425 (3 μM), and CD11a mRNA levels were analyzed at indicated times post pretreatment (*=p<0.05 vs. DMSO-treated control MSCs, Tukey's HSD test).

FIG. 9A demonstrates that CD11a expression on MSC surface upon compound pretreatment is stable for up to 4 days. MSCs were pretreated with DMSO vehicle control (0.1%) or Ro-31-8425 (3 μM) for 24 h. The CD11a expression levels were assessed via CyTOF analysis on day 1 and day 4 post pretreatment (upper section of dots, CD11a+ MSCs; lower section of dots, CD11a-MSCs).

FIG. 10 illustrates that compound pretreatment upregulates CD11a expression on MSCs from different donors. CD11a surface expression on MSCs from multiple different donors in response to pretreatment with compound Ro-31-8425. MSCs were pretreated with DMSO vehicle control (0.1%) or Ro-31-8425 (3 μM) for 24 h, and CD11a expression levels were assessed via CyTOF analysis (*=p<0.05 versus DMSO-treated control MSCs, Tukey's HSD test).

Pretreatment of MSCs enhanced firm adhesion to ICAM-1 under dynamic flow conditions.

We next assessed the effect of the identified CD11a-upregulating hits on MSC firm adhesion, which is part of the leukocyte adhesion cascade and is also governed by CD11a. CD11a is known to mediate leukocyte firm adhesion with endothelial cells via interaction with Intercellular Adhesion Molecules (ICAMs), and specifically ICAM-1. Therefore, we tested firm adhesion of pretreated MSCs to ICAM-1, which is upregulated on the endothelial surface at sites of inflammation and is involved in leukocyte recruitment during inflammation. The results showed that pretreatment of MSCs with compound Ro-31-8425 enhanced MSC firm adhesion to an ICAM-1-coated surface under dynamic flow conditions.

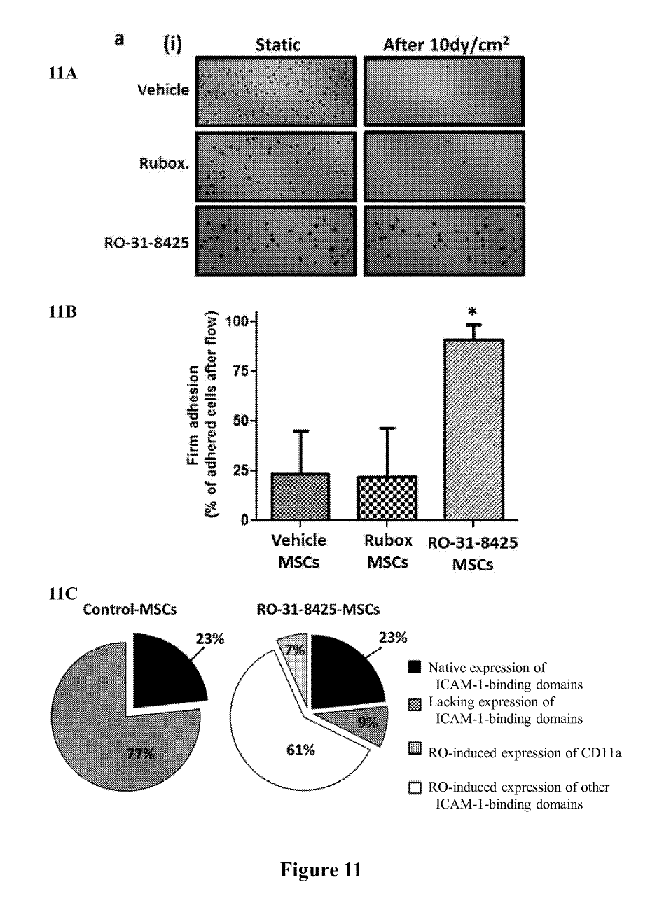

MSCs were incubated with a given compound and then subjected to a firm adhesion assay under physiologically relevant shear flow using a multiwell plate microfluidic system. Pretreatment with compound Ro-31-8425, which up-regulated CD11a expression, also induced a >3-fold increase in MSC firm adhesion to an ICAM-1-coated substrate compared to control, vehicle-treated MSCs. See FIGS. 11A and 11B. As depicted in FIG. 11C, Ro-31-8425 pretreatment induced ICAM-1 firm adhesion of a new MSC sub-population comprising 68% of the entire population, out of which approximately 7% are CD11a+, consistent with data from FIG. 8A. The rest (61%) of the subpopulation express other ICAM-1-binding domains. In contrast, pretreatment with the protein kinase C inhibitor ruboxistaurin, which did not increase MSC CD11a expression, also did not improve MSC firm adhesion to ICAM-1-coated substrates.

FIG. 11 illustrates that upregulation of CD11a, in response to pretreatment with Ro-31-8425, increases MSC firm adhesion to an ICAM-1-coated surface in vitro. FIG. 11A shows results of MSCs firm adhesion to an ICAM-1-coated surface following pretreatment with ruboxistaurin (Rubox) or Ro-31-8425 (3 μM for 24 h, 10× magnification). FIG. 11B shows a quantification of MSC firm adhesion to an ICAM-1 surface in response to pretreatment with ruboxistaurin or Ro-31-8425. Error bars represent the standard deviation (statistically significant difference vs. vehicle-treated control is denoted by *=p<0.05, Tukey's HSD test). FIG. 11C shows a pie chart of the percent distribution of MSC population that express ICAM-1 binding domains following Ro-31-8425 pretreatment.

### Antibody Blocking Studies and the Role of CD11a in ICAM-1 Binding for Firm Adhesion.

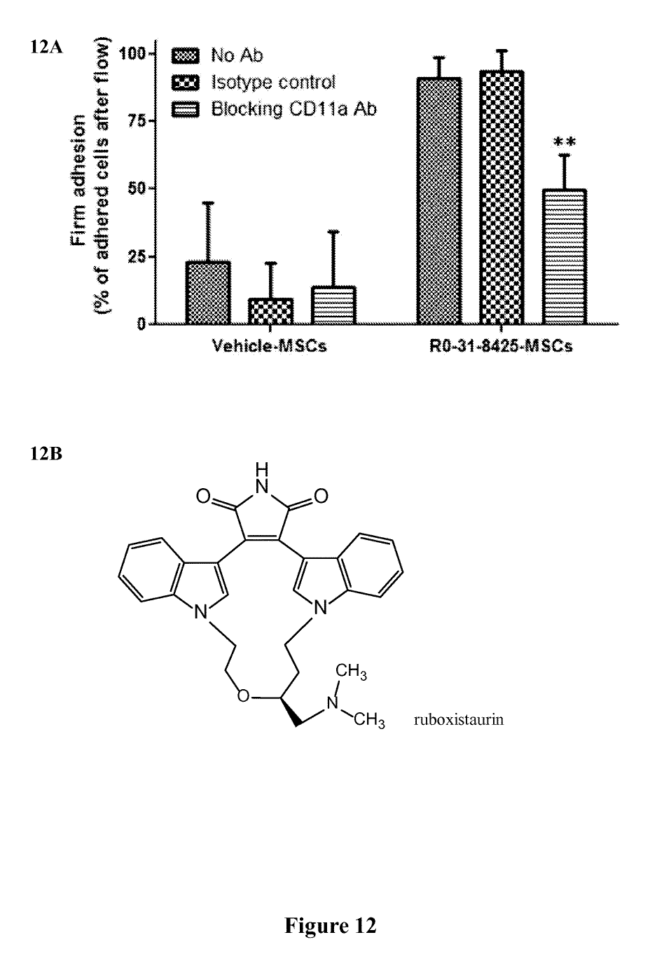

To explore the possible involvement of CD11a in mediating firm adhesion by pretreated MSC to an ICAM-1-coated surface, we performed Ab blocking experiments. See FIG. 12B. Incubating with CD11a blocking Ab significantly reduced Ro-31-8425-pretreated MSC firm adhesion to ICAM-1-coated surface (a reduction from 90% of adhered cells to 50% following CD11a blocking). This data suggests that CD11a, which was upregulated in response to Ro-31-8425 pretreatment, is involved in mediating the increased MSC firm adhesion to ICAM-1. However, CD11a blocking did not fully abolish Ro-31-8425-pretreated MSC firm adhesion to control untreated MSC levels, further suggesting that other ICAM-1-binding ligands are also involved in mediating the increased firm adhesion of Ro-31-8425-treated MSCs to ICAM-1.

FIG. 12 shows results of antibody blocking experiments. These results demonstrate a significant involvement of CD11a in the increased firm adhesion of Ro-31-8425-treated MSCs to an ICAM-1 surface. Error bars represent standard deviation (statistically significant difference versus no Ab control and versus CD90 Ab control is denoted by **=p<0.05, Tukey's HSD test).

In an embodiment of the invention, pretreatment of MSCs with a compound enhances an ICAM-1 binding activity. In a particular embodiment, the ICAM-1 binding activity is mediated by an interaction involving an MSC with increased CD11a surface expression. In another embodiment, the ICAM-1 binding activity is mediated by an interaction involving an MSC surface molecule other than CD11a.

### In Vivo Anti-Inflammatory Therapeutic Effect of Pretreated MSCs.

We further assessed whether MSCs pretreated with compounds were able to mediate a significant anti-inflammatory therapeutic effect in vivo. We found that Ro-31-8425-preconditioned MSCs home efficiently to inflamed sites and exhibit an improved anti-inflammatory impact following systemic administration in a mammalian system.

Compounds that significantly increased MSC firm adhesion to ICAM-1 in vitro were tested in vivo for the ability to promote targeting of systemically administered MSCs to a distant site of inflammation. In our murine model, one ear pinna was injected with LPS to induce local inflammation, while the other received a saline injection. This model was previously established to evaluate several MSC bioengineering strategies and has good sensitivity.

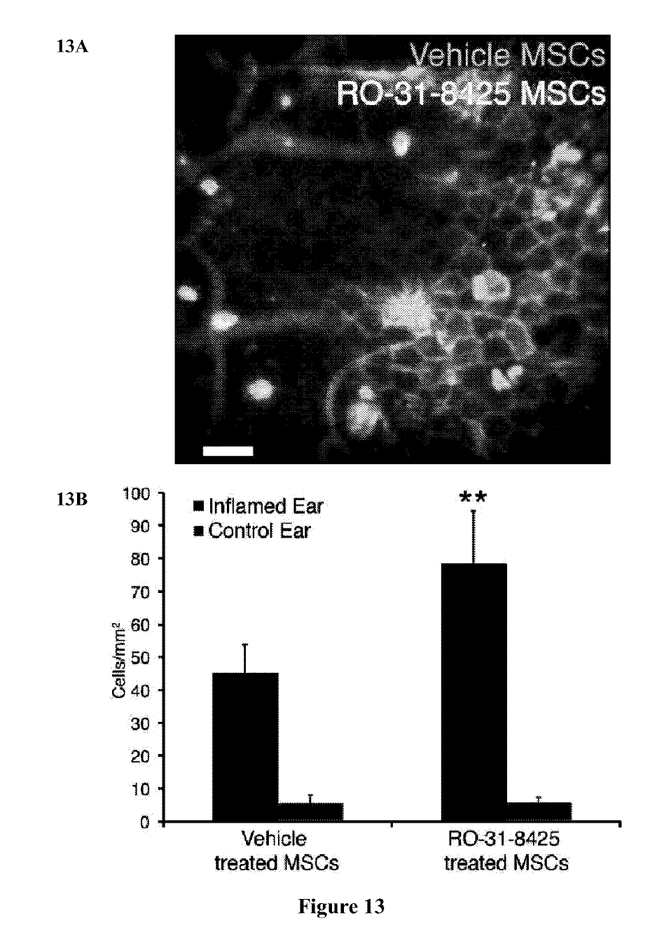

Briefly, compound-treated and vehicle-treated MSCs (stained with different membrane tracker dyes and mixed at 1:1 ratio) were systemically infused into mice. Cell homing to the inflamed and control ears was imaged 24 h later using intravital microscopy. FIG. 13A shows a microscopic image of MSCs in situ (left-facing white arrows, compound-treated using RO-31-8425; right-facing hatched arrows, vehicle-treated). Pretreatment with Ro-31-8425 significantly improved MSC homing to skin in the inflamed ear upon systemic administration, with an average of 45.2±8.6 cells per cubic mm for vehicle-treated MSCs and 78.5±15.9 cells/mm³ for Ro-31-8425-treated MSCs (69.3±11.3% increase for compound-treated compared to vehicle-treated MSCs). This data demonstrates a strong relationship between surface expression of CD11a, ICAM-1 firm adhesion, and homing of systemically transplanted MSCs to sites of inflammation.

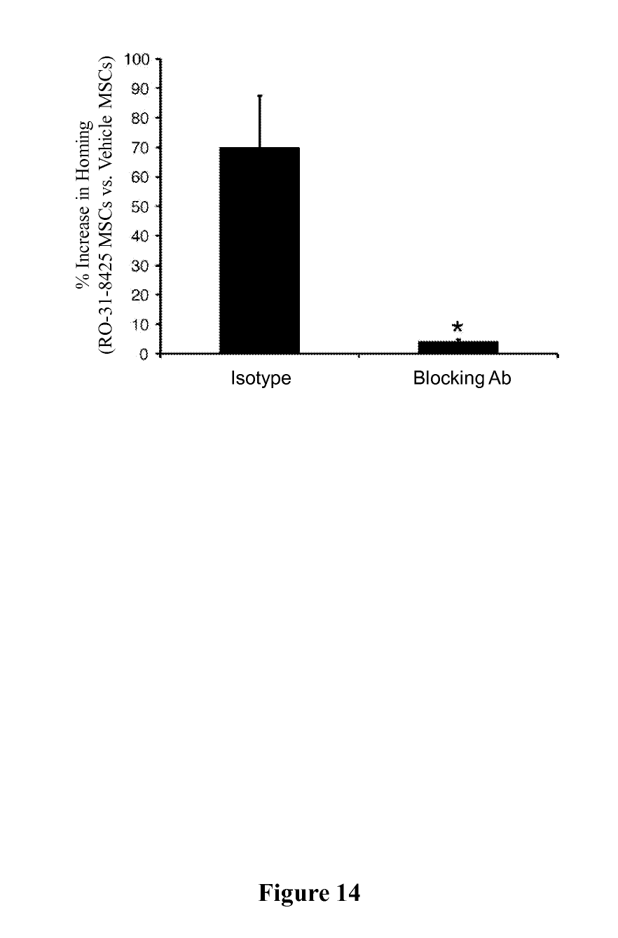

Furthermore, when CD11a was blocked on Ro-31-8425-pretreated MSCs prior to systemic infusion, their enhanced homing response to the site of inflammation was reversed, dropping from 70% to less than 10% increased homing versus vehicle-treated MSCs (FIG. 14). These results further implicate CD11a and other ICAM-1 binding domains that mediate the enhanced homing response of systemically infused Ro-31-8425-pretreated MSCs to sites of inflammation.

We assessed the ability of Ro-31-8425-pretreated MSCs, which exhibited increased homing to the inflamed ear, to alleviate the severity of LPS-induced local inflammation. To evaluate ear inflammation, ear thickness and local levels of the pro-inflammatory cytokine TNF-α in mice ears were measured 24 h post-administration of either vehicle or Ro-31-8425-pretreated MSCs. As shown in FIG. 3C, while mice treated with vehicle control MSCs exhibited a small reduction in ear thickness (6.3±5.2 μm reduction compared to no MSC treatment), MSCs pre-treated with Ro-31-8425 exhibited a greater than 3-fold effect in reducing ear swelling (20.0±5.3 μm reduction). LPS-induced inflammation resulted not only in ear swelling but also in a significant increase in local levels of the pro-inflammatory cytokine TNF-α in the inflamed ear compared to the saline-treated ear (4.5±1.3 fold TNF-α increase in the inflamed ear vs. control ear, FIG. 3D). Consistent with the cell delivery and ear thickness data, the increased TNF-α levels in the inflamed ear were significantly reduced (˜50%) by administration of Ro-31-8425-treated MSCs, whereas vehicle-treated MSCs did not impact TNF-α levels. Taken together, these results show that systemic infusion of Ro-31-8425-pretreated MSCs, which display CD11a and other ICAM-1 binding domains, not only increased homing to inflamed tissues but also provided an improved anti-inflammatory therapeutic effect in vivo.

FIG. 13 illustrates that Ro-31-8425-pretreated MSCs exhibited increased homing to inflamed sites and an improved anti-inflammatory impact following systemic administration. Homing of systemically infused MSCs to LPS-induced inflamed mouse ears was assessed 24 hr following cell infusion. FIG. 13A shows example images (scale bar=25 μm) demonstrating homing to the inflamed ear of Ro-31-8425 pre-treated MSCs (green cells, left-facing white arrows) compared to vehicle-treated MSCs (blue cells, right-facing hatched arrows). As shown more quantitatively in FIG. 13B, Ro-31-8425 pretreatment significantly promoted MSC homing versus the vehicle-treated control cells (**=p<0.01, Tukey's HSD test, n=8 mice).

FIG. 14 illustrates the results of antibody blocking experiments in the context of an in vivo homing study. For antibody blocking experiments, pretreated or control MSCs were washed and incubated for 30 minutes with mouse anti-human CD11a (clone TS1/22) or Mouse IgG1 isotype control prior to staining with the lipophilic membrane dyes and retro-orbital infusion. The Ab blocking experiments demonstrated involvement of CD11a and other ICAM-1 binding domains in the increased homing response of systemically infused Ro-31-8425-treated MSCs to the inflamed ear. CD11a-blocked or Ab isotype control-incubated Ro-31-8425-pretreted MSCs were co-injected systemically with vehicle MSCs (1:1 ratio), and the homing response to inflamed ear was assessed via intravital microscopy. Error bars represent standard deviation (statistically significant difference versus Ab isotype control is denoted by *=p<0.05, Tukey's HSD test, n=5 mice per group).

FIG. 3C illustrates that Ro-31-8425-treated MSCs displayed a superior effect in reducing swollen ear thickness of the inflamed ear compared to native MSCs (*=p<0.05, **=p<0.01, Tukey's HSD test, n=8 mice). FIG. 3D shows that MSCs treated with Ro-31-8425 significantly reduced the TNF-α level in the inflamed ear compared to the control ear (**=p<0.01, Tukey's HSD test, n=6 mice).

Effect of Ro-31-8425 on Cell Viability of MSCs.

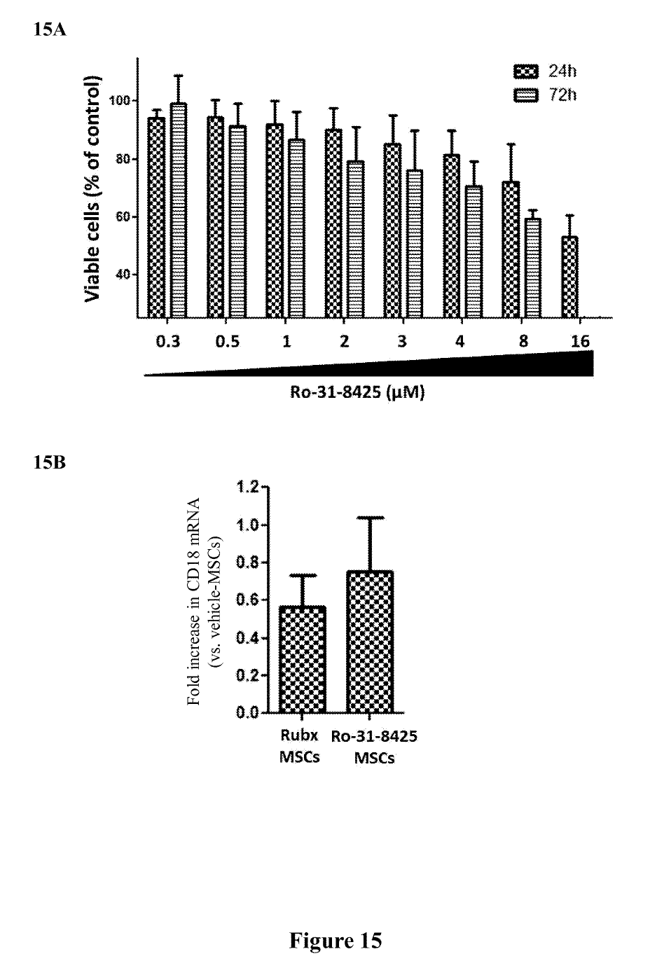

Evaluation of MSC viability demonstrated that Ro-31-8425 did not significantly compromise cell viability at concentrations of 0.25 to 4 μM following a 24 h pretreatment. FIG. 15A shows that the compound exhibited toxicity to MSCs in vitro only at concentration levels greater than 4 μM post 72 h pretreatment of MSCs. MSCs were pretreated with Ro-31-8425 for 24 h or 72 h, followed by quantification of MSC viability via XTT. (Y axis presents the percentage of viable cells compared to untreated control (cells incubated with 10% FBS-supplemented MEMa media), error bars represent standard deviation, n=3).

Effect of Ro-31-8425 on Levels of CD18 mRNA Expression by MSCs.

The molecule CD18, also known as integrin β2, is known to pair with CD11a to form LFA-1. We observed CD18 mRNA levels for MSCs in response to compound Ro-31-8425 and ruboxistaurin pretreatment as analyzed by RT-PCR. The compound Ro-31-8425 did not upregulate mRNA levels of CD18; results are shown in FIG. 15B. MSCs were pretreated with Ro-31-8425 (3 μM) or Ruboxistaurin (Rubx, 3 μM) and CD18 mRNA levels were analyzed at indicated times post pretreatment (n=3, data presented as fold increase vs. DMSO-treated control MSCs).

mRNA Analysis of CD11a and CD18.

The mRNA levels of CD11a and CD18 in response to Ro-31-8425 pretreatment of MSCs were analyzed by qPCR. Specifically, MSCs were treated with Ro-31-8425 (3 μM) or vehicle control (0.1% DMSO) for 2 h, 4 h, 8 h, 14 h or 24 h. Cells were then trypsinized, washed with ice-cold PBS and pelleted (500 g for 5 min at 4° C.) at during the treatment and immediately stored at −80° C. RNA extraction was then performed. Briefly, the frozen cell pellet was crushed with a tissue pestle (Fisher Scientific) in the presence of 1 mL TRI RNA isolation reagent (Sigma Aldrich). Upon complete pellet dissociation, 0.2 mL chloroform was added and suspension was vortexed for 15 s, followed by incubation at ambient temperature for 10 min. The mixture was centrifuged at 15,000 g for 15 min at 40° C., the resulting supernatant aqueous phase was removed, thoroughly mixed with 0.5 mL isopropanol (Sigma Aldrich) and further centrifuged (at 15,000 g for 10 min) to obtain the total RNA precipitates. The RNA pellets were washed twice in 75% ethanol, and air dried for 10 min at room temperature before being reconstituted with 20 uL ultrapure H₂O. The quality and quantity of the total RNAs were measured and verified using Nanodrop ND-2000 Spectrophotometer (NanoDrop Products). Subsequently, equal amount of total RNA (2 μg) was reverse transcribed into cDNA using a QuantiTect reverse transcription kit (Qiagen). Then the qPCR reaction was performed using an ABI 7900HT Sequence Detection System (Applied Biosystems), with a reaction volume of 15 μL (10 ng/μL cDNA, 2 μM primers, and 7.5 μL Power SYBR green PCR master mix (Life Technology)). Peptidylprolyl isomerase A (PPIA) was selected as internal reference gene, and the sequences of its forward and reverse primers were referenced. Sequences of the CD11α and CD18 primers (gene accession numbers: NM_002209 and NM_000211, respectively) were designed using Primer3 software (http ifrodo.wi.mit.edu/primer3). Primers used for CD11a: 5′-CAGGCTATTTGGGTTACACCG-3′ (sense), SEQ ID NO:1; and 5′-CCATGTGCTGGTATCGAGGG-3′ (anti-sense), SEQ ID NO:2; and for CD18: 5′-TGCGTCCTCTCTCAGGAGTG-3′ (sense), SEQ ID NO:3; and 5′-GGTCCATGATGTCGTCAGCC-3′ (anti-sense), SEQ ID NO:4. The oligomers were manufactured by Integrated DNA Technologies. The relative expression (fold change) was normalized to the vehicle control groups at respective time points using 2ΔΔCt method.

Additional Secretome Analysis.

Upon further analysis, we observed that Ro-31-8425 did not substantially alter the MSC secretome. Out of 48 secreted factors tested via Ab-based multiplex assays, only 3 analytes (IL-6, MCP-1 and VEGF), showed statistically significant changes in response to Ro-31-8425 pretreatment. See Table 5.

TABLE 5
Results of additional secretome analysis for pretreated MSCs.

| Parameter | IL-6 | MCP-1 | VEGF |
|---|---|---|---|
| Fold change of | 0.34 +/− 0.09 | 0.54 +/− 0.09 | 0.75 +/− 0.26 |
| analyte upon | | | |
| Ro-31-8425 | | | |
| pretreatment (vs. | | | |
| vehicle control) | | | |

Further Secretome Analysis.

Analysis of the secretome of MSCs was conducted upon pretreatment of MSCs with compound 1929 for 24 h. In this experiment, the MSCs were from donor 318006 (BM318006 P7). The mean and standard deviation values of secretome analytes were measured in pg/ml. The donor Results for certain analytes are shown in Table 6.

TABLE 6
Secretome analysis for pretreated MSCs.

| | Pretreatment condition | | | Pretreatment condition | | |
|---|---|---|---|---|---|---|
| | | 1929 | 1929 | | 1929 | 1929 |
| | DMSO | 0.3 μM | 3 μM | DMSO | 0.3 μM | 3 μM |
| Analyte | Mean of [Analyte], pg/ml | | | Standard Deviation | | |
| IL-1ra | 21.7 | 16.5 | 22.6 | 1.7 | 1.7 | 1.2 |
| IL-8 | 30.8 | 26.9 | 31.1 | 0.3 | 1.5 | 0.9 |
| IL-12p70 | 14.3 | 13.3 | 13.9 | 1.3 | 0.3 | 0.5 |
| LIF | 17.2 | 14.2 | 10.8 | 1.2 | 1.3 | 0.5 |
| MIF | 71.2 | 54.9 | 69.4 | 2.4 | 7.2 | 2.4 |
| IFN-g | 15.5 | 11.0 | 14.9 | 0.2 | 0.8 | 0.0 |
| MCP-1 | 30.0 | 26.4 | 22.5 | 0.0 | 1.1 | 0.8 |
| IL-6 | 698.3 | 451.3 | 333.5 | 1.3 | 2.3 | 6.1 |
| VEGF | 615.7 | 547.2 | 486.8 | 5.8 | 2.8 | 19.4 |

### Experimental Procedures

Donor Source Material.

MSCs were purchased from Lonza (donors used were designated 7F3915, 318006 and 351482).

### SEQUENCE LISTING

Any sequence listing information is considered part of this application.

CyTOF Analysis for Assessing CD11a Expression Levels.

To further confirm the screening results, the surface expression of CD11a was also examined by Time of Flight Mass Cytometry (CyTOF2, DVS Sciences) (Newell et al., 2013, Nat Biotechnol. 31(7):623-9). MSCs were treated with Ro-31-8425 as indicated and sample preparation was performed per manufacturer's instructions with slight modifications. Briefly, the harvested MSC pellet was first resuspended with Intercalator-103Rh solution (1 μM) in PBS to identify viable cells, then washed twice with MaxPar cell staining buffer and stained with 100 μL antibody cocktail (human Nd142-CD11a and Tb159-CD90, 1:200 dilution in staining buffer) for 30 min at room temperature. After two washes, the cells were further stained with 125 nM MaxPar Intercalator-Ir in MaxPar Fix and Perm buffer for 1-2 h with gentle shaking. Finally the fixed cells were thoroughly washed with ultrapure H2O 2O for three times (1000 g, 5 min each), resuspended in 300 μL H2O and filtered through a 40 μm Falcon cell strainer (Corning) prior to CyTOF data acquisition. The CyTOF data was analyzed with Cytobank on-line data analysis platform (website www cytobank.org).

Culture Expansion and Preparation of Pretreated MSCs.

In embodiments of the invention, quantities of materials are prepared such as for therapeutic use. In an embodiment, an amplification of MSCs is performed prior to pretreatment with a compound capable of enhancing a homing function. In such embodiment, the MSCs are expanded in culture in vitro. As an integral part of the culture expansion or as a separate process following expansion, the cells are subject to a pretreatment regimen. The pretreated MSCs are then chilled or frozen for temporary or long term storage. The stored pretreated MSCs are thawed prior to use. In an embodiment, such use can be for a research purpose and/or for a therapeutic administration to a patient in need of treatment. Thus embodiments of compositions of homing-enhanced MSCs are prepared with the practical advantage whereby a patient treating facility is not required to conduct the pretreatment process of the MSCs. In another embodiment, culture expanded MSCs are frozen and optionally stored frozen for a period of time, followed by thawing and pretreatment for enhanced homing as described according to this disclosure.

Statements Regarding Incorporation by Reference and Variations.

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

When a group of substituents is disclosed herein, it is understood that all individual members of that group and all subgroups, including any isomers, enantiomers, and diastereomers of the group members, are disclosed separately.

When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomer and enantiomer of the compound described individual or in any combination. Additionally, unless otherwise specified, all isotopic variants of compounds disclosed herein are intended to be encompassed by the disclosure. As a brief illustration, it will be understood that any one or more hydrogens in a molecule disclosed can be replaced with deuterium or tritium.

Isotopic variants of a molecule are generally useful as standards in assays for the molecule and in chemical and biological research related to the molecule or its use. Methods for making such isotopic variants are known in the art.

As used herein, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “containing” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the pertinent art.

Whenever a range of values is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

One of ordinary skill in the art will appreciate that starting materials, biological and chemical materials, biological and chemical reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this disclosure.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by various embodiments which may include preferred embodiments, exemplary embodiments and optional features, modifications and variations of the concepts herein disclosed may be resorted to by those skilled in the art. Such modifications and variations are considered to be within the scope of this invention as defined by the appended claims.

1. A method of treating a subject with a disease or injury condition, the method comprising the steps of:
(a) providing cells in vitro;
(b) contacting the cells with an effective amount of a compound composition comprising a compound, thereby generating a composition comprising pretreated cells, wherein the compound is capable of improving a homing function in pretreated cells relative to that of untreated cells, wherein said contacting optionally includes incubating the cells with the compound composition; and
wherein the compound has structure of formula I: 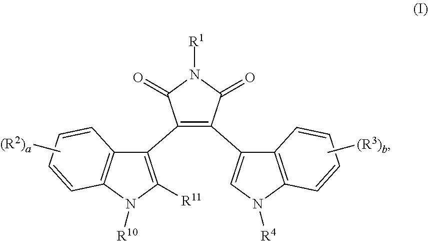
wherein
R¹ is selected from the series consisting of hydrogen, (C₁-C₄)-alkyl, phenyl-(C₁-C₄)-alkyl- and heteroaryl-(C₁-C₄)-alkyl-, wherein phenyl and heteroaryl are unsubstituted or substituted by substituents from the series consisting of halogen, (C₁-C₄)-alkyl and (C₁-C₄)-alkyl-O—;
R² and R³ are independently of each other selected from the series consisting of halogen, (C₁-C₄)-alkyl and (C₁-C₄)-alkyl-O—;
R⁴ is selected from the series consisting of hydrogen and (C₁-C₄)-alkyl;
R¹⁰ is selected from the series consisting of hydrogen, (C₁-C₆)-alkyl and Het¹, wherein (C₁-C₆)-alkyl is unsubstituted or substituted by R²⁰, and wherein Het¹ is unsubstituted or substituted by R²¹;
R¹¹ is selected from the series consisting of hydrogen and (C₁-C₄)-alkyl,
or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl;
R²⁰ is selected from the series consisting of R³⁰—O—, R³¹—N(R³²)—, H₂N—C(═NH)—S—, pyridinyl and Het², wherein Het² is unsubstituted or substituted by R³³;
R²¹ is selected from the series consisting of (C₁-C₄)-alkyl, phenyl-(C₁-C₄)-alkyl- and pyridinyl-(C₁-C₄)-alkyl-, wherein phenyl and pyridinyl are unsubstituted or substituted by substituents from the series consisting of halogen, (C₁-C₄)-alkyl and (C₁-C₄)-alkyl-O—;
R²² is selected from the series consisting of hydrogen and (C₁-C₄)-alkyl;
R²³ is selected from the series consisting of hydrogen, (C₁-C₄)-alkyl, R³¹—N(R³²)— and R³¹—N(R³²)—(C₁-C₄)-alkyl-;
R²⁴ is selected from the series consisting of hydrogen, (C₁-C₄)-alkyl, pyridinyl-(C₁-C₄)-alkyl- and R³¹—N(R³²)—(C₁-C₄)-alkyl-;
R³⁰, R³¹ and R³² are independently of each other selected from the series consisting of hydrogen and (C₁-C₄)-alkyl;
R³³ is selected from the series consisting of (C₁-C₄)-alkyl;
Het¹ is a 4-membered to 7-membered, monocyclic, saturated heterocycle comprising one ring nitrogen atom, which is bonded via a ring carbon atom;
Het² is a 4-membered to 7-membered, monocyclic, saturated heterocycle comprising one or two ring nitrogen atoms, which is bonded via a ring carbon atom or a ring nitrogen atom;
heteroaryl is a 5-membered or 6-membered, monocyclic, aromatic heterocycle comprising one or two identical or different ring heteroatoms selected from the series consisting of N, O and S;
a and b are independently of each other selected from the series consisting of 0, 1 and 2;
m and n are independently of each other selected from the series consisting of 1 and 2;
(b′) optionally washing the pretreated cells; and
(c) administering an effective amount of the composition comprising pretreated cells to the subject.

2. The method of claim 1, wherein in the compound of formula I,
R¹ is selected from the series consisting of hydrogen, (C₁-C₄)-alkyl and phenyl-(C₁-C₄)-alkyl-, wherein phenyl is unsubstituted or substituted by substituents from the series consisting of halogen, (C₁-C₄)-alkyl and (C₁-C₄)-alkyl-O—;
R² and R³ are independently of each other selected from the series consisting of halogen, (C₁-C₄)-alkyl and (C₁-C₄)-alkyl-O—;
R⁴ is selected from the series consisting of hydrogen and (C₁-C₄)-alkyl;
R¹⁰ is selected from the series consisting of (C₁-C₆)-alkyl and Het¹, wherein (C₁-C₆)-alkyl is unsubstituted or substituted by R²⁰, and wherein Het¹ is unsubstituted or substituted by R²¹;
R¹¹ is selected from the series consisting of hydrogen and (C₁-C₄)-alkyl,
or R¹⁰ and together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl;
R²⁰ is selected from the series consisting of R³¹—N(R³²)—, H₂N—C(═NH)—S—, pyridinyl and Het², wherein Het² is unsubstituted or substituted by R³³;
R²¹ is selected from the series consisting of (C₁-C₄)-alkyl, phenyl-(C₁-C₄)-alkyl- and pyridinyl-(C₁-C₄)-alkyl-, wherein phenyl and pyridinyl are unsubstituted or substituted by substituents from the series consisting of halogen, (C₁-C₄)-alkyl and (C₁-C₄)-alkyl-O—;
R²² is selected from the series consisting of hydrogen and (C₁-C₄)-alkyl;
R²³ is selected from the series consisting of hydrogen, (C₁-C₄)-alkyl, R³¹—N(R³²)— and R³¹—N(R³²)—(C₁-C₄)-alkyl-;
R²⁴ is selected from the series consisting of hydrogen, (C₁-C₄)-alkyl, pyridinyl-(C₁-C₄)-alkyl- and R³¹—N(R³²)—(C₁-C₄)-alkyl-;
R³¹ and R³² are independently of each other selected from the series consisting of hydrogen and (C₁-C₄)-alkyl;
R³³ is selected from the series consisting of (C₁-C₄)-alkyl;
Het¹ is a 4-membered to 7-membered, monocyclic, saturated heterocycle comprising one ring nitrogen atom, which is bonded via a ring carbon atom;
Het² is a 4-membered to 7-membered, monocyclic, saturated heterocycle comprising one or two ring nitrogen atoms, which is bonded via a ring carbon atom or a ring nitrogen atom;
a and b are independently of each other selected from the series consisting of 0 and 1;
m and n are independently of each other selected from the series consisting of 1 and 2.

3. The method of claim 1, wherein in the compound of formula I,
R¹ is selected from the series consisting of hydrogen, (C₁-C₄)-alkyl and phenyl-(C₁-C₄)-alkyl-;
R² and R³ are independently of each other selected from the series consisting of halogen, (C₁-C₄)-alkyl and (C₁-C₄)-alkyl-O—;
R⁴ is selected from the series consisting of hydrogen and (C₁-C₄)-alkyl;
R¹⁰ is selected from the series consisting of (C₁-C₆)-alkyl and Het¹, wherein (C₁-C₆)-alkyl is unsubstituted or substituted by R²⁰, and wherein Het¹ is unsubstituted or substituted by R²¹;
R¹¹ is selected from the series consisting of hydrogen and (C₁-C₄)-alkyl,
or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl;
R²⁰ is selected from the series consisting of R³¹—N(R³²)—, H₂N—C(═NH)—S— and Het², wherein Het² is unsubstituted or substituted by R³³;
R²¹ is selected from the series consisting of (C₁-C₄)-alkyl and pyridinyl-(C₁-C₄)-alkyl-, wherein pyridinyl is unsubstituted or substituted by substituents from the series consisting of halogen, (C₁-C₄)-alkyl and (C₁-C₄)-alkyl-O—;
R²² is hydrogen;
R²³ is selected from the series consisting of hydrogen, (C₁-C₄)-alkyl, R³¹—N(R³²)— and R³¹—N(R³²)—(C₁-C₄)-alkyl-;
R²⁴ is selected from the series consisting of hydrogen, (C₁-C₄)-alkyl and pyridinyl-(C₁-C₄)-alkyl-;
R³¹ and R³² are independently of each other selected from the series consisting of hydrogen and (C₁-C₄)-alkyl;
R³³ is selected from the series consisting of (C₁-C₄)-alkyl;
Het¹ is a 5-membered or 6-membered, monocyclic, saturated heterocycle comprising one ring nitrogen atom, which is bonded via a ring carbon atom;
Het² is a 4-membered to 6-membered, monocyclic, saturated heterocycle comprising one or two ring nitrogen atoms, which is bonded via a ring carbon atom or a ring nitrogen atom;
a and b are independently of each other selected from the series consisting of 0 and 1;
m and n are independently of each other selected from the series consisting of 1 and 2.

4. The method of claim 1, wherein in the compound of formula I,
R¹ is hydrogen;
R⁴ is selected from the series consisting of hydrogen and (C₁-C₄)-alkyl;
R¹⁰ is selected from the series consisting of (C₁-C₄)-alkyl and Het¹, wherein (C₁-C₄)-alkyl is unsubstituted or substituted by R²⁰, and wherein Het¹ is unsubstituted or substituted by R²¹;
R¹¹ is hydrogen,
or R¹⁰ and R¹¹ together are a divalent group selected from the series consisting of the groups —(CH₂)ₘ—C(R²²)(R²³)—(CH₂)ₙ— and —(CH₂)ₘ—N(R²⁴)—(CH₂)ₙ—;
R²⁰ is selected from the series consisting of R³¹—N(R³²)—, H₂N—C(═NH)—S— and Het², wherein Het² is unsubstituted or substituted by R³³;
R²¹ is selected from the series consisting of pyridinyl-(C₁-C₄)-alkyl-;
R²² is hydrogen;
R²³ is selected from the series consisting of R³¹—N(R³²)— and R³¹—N(R³²)—(C₁-C₄)-alkyl-;
R²⁴ is selected from the series consisting of hydrogen and (C₁-C₄)-alkyl;
R³¹ and R³² are independently of each other selected from the series consisting of hydrogen and (C₁-C₄)-alkyl;
R³³ is selected from the series consisting of (C₁-C₄)-alkyl;
Het¹ is a 5-membered or 6-membered, monocyclic, saturated heterocycle comprising one ring nitrogen atom, which is bonded via a ring carbon atom;
Het² is a 4-membered to 6-membered, monocyclic, saturated heterocycle comprising one or two ring nitrogen atoms, which is bonded via a ring carbon atom or a ring nitrogen atom;
a and b are 0;
m is 2 and n is 1.

5. The method of claim 1, wherein in the compound of formula I,
R¹ is hydrogen;
R⁴ is selected from the series consisting of hydrogen and (C₁-C₄)-alkyl;
R¹⁰ is selected from the series consisting of (C₁-C₄)-alkyl, wherein (C₁-C₄)-alkyl is unsubstituted or substituted by R²⁰;
R¹¹ is hydrogen,
or R¹⁰ and R¹¹ together are a divalent group selected from the series consisting of the groups —(CH₂)ₘ—C(R²²)(R²³)—(CH₂)ₙ—;
R²⁰ is selected from the series consisting of R³¹—N(R³²)—, H₂N—C(═NH)—S— and Het², wherein Het² is unsubstituted or substituted by R³³;
R²² is hydrogen;
R²³ is selected from the series consisting of R³¹—N(R³²)— and R³¹—N(R³²)—(C₁-C₄)-alkyl-;
R³¹ and R³² are independently of each other selected from the series consisting of hydrogen and (C₁-C₄)-alkyl;
R³³ is selected from the series consisting of (C₁-C₄)-alkyl;
Het² is a 5-membered to 6-membered, monocyclic, saturated heterocycle comprising one or two ring nitrogen atoms, which is bonded via a ring carbon atom or a ring nitrogen atom;
a and b are 0;
m is 2 and n is 1.

6. The method of claim 1, wherein in the compound of formula I,
R¹ is hydrogen;
R⁴ is selected from the series consisting of hydrogen and (C₁-C₄)-alkyl;
R¹⁰ is selected from the series consisting of (C₁-C₄)-alkyl, wherein (C₁-C₄)-alkyl is unsubstituted or substituted by R²⁰;
R¹¹ is hydrogen,
or R¹⁰ and R¹¹ together are a divalent group selected from the series consisting of the groups —(CH₂)ₘ—C(R²²)(R²³)—(CH₂)ₙ—;
R²⁰ is selected from the series consisting of R³¹—N(R³²)— and Het², wherein Het² is unsubstituted or substituted by R³³;
R²² is hydrogen;
R²³ is selected from the series consisting of R³¹—N(R³²)—(C₁-C₄)-alkyl-;
R³¹ and R³² are independently of each other selected from the series consisting of hydrogen and (C₁-C₄)-alkyl;
R³³ is selected from the series consisting of (C₁-C₄)-alkyl;
Het² is a 5-membered to 6-membered, monocyclic, saturated heterocycle comprising one ring nitrogen atom, which is bonded via a ring carbon atom;
a and b are 0;
m is 2 and n is 1.

7. The method of claim 1, wherein in the compound of formula I,
R¹ is hydrogen;
R⁴ is selected from the series consisting of (C₁-C₄)-alkyl;
R¹⁰ and R¹¹ together are a divalent group selected from the series consisting of the groups —(CH₂)ₘ—C(R²²)(R²³)—(CH₂)ₙ—;
R²² is hydrogen;
R²³ is selected from the series consisting of R³¹—N(R³²)—(C₁-C₄)-alkyl-;
R³¹ and R³² are independently of each other selected from the series consisting of hydrogen and (C₁-C₄)-alkyl;
a and b are 0;
m is 2 and n is 1.

8. The method of claim 1, wherein in the compound of formula I, the compound chemical name is 3-(8-aminomethyl-6,7,8,9-tetrahydro-pyrido[1,2-a]indol-10-yl)-4-(1-methyl-1H-indol-3-yl)-pyrrole-2,5-dione; and has structure of formula I-1:

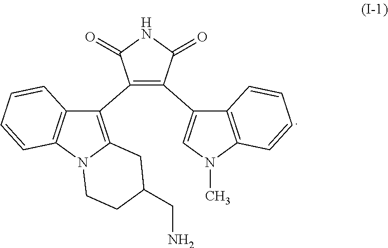 [SMILES: Cn1cc(C2=C(c3c4n(c5ccccc35)CCC(CN)C4)C(=O)NC2=O)c2ccccc21]

9. The method of claim 1, wherein the cells are mesenchymal stem cells (MSCs); stem/progenitor cells including skeletal muscle-derived stem/progenitor cells (MDSPCs), satellite cells, hematopoietic stem/progenitor cells, bone or bone marrow derived stem/progenitor cells, neural stem/progenitor cells, eye stem/progenitor cells, liver derived stem-progenitor cells, brain derived stem/progenitor cells, heart/cardiac derived stem/progenitor cells, intestinal stem/progenitor cells, mesenchymal stem/progenitor cells, skin stem/progenitor cells, hair/hair follicle stem/progenitor cells, endothelial stem/progenitor cells, epithelial stem/progenitor cells, olfactory adult stem/progenitor cells, neural crest stem/progenitor cells, testicular stem/progenitor cells, embryonic stem cells, placental derived stem/progenitor cells, amniotic fluid-derived stem/progenitor cells, mucosal stem/progenitor cells, cord blood stem/progenitor cells, LGRS+ stem or progenitor cells, and inducible pluripotent stem cells; progeny cells of the foregoing; or modified or engineered cells of the foregoing; the cells can further include any of: cells derived from islets including but not limited to beta cells, delta cells, alpha cells, acinar cells; programmed and reprogrammed cells and their progeny; mesenchymal stem cells derived from cells that have been reprogrammed to progenitors or stem cells or programmed directly to MSCs; osteochondroreticular stem/progenitor cells; connective tissue progenitor cells; and multipotent adult progenitor cells.

10. The method of claim 1, wherein the disease or injury condition is an inflammatory condition.

11. The method of claim 1, wherein the cells are MSCs.

12. The method of claim 1, wherein the cells are mammalian MSCs.

13. The method of claim 1, wherein the cells are human MSCs.

14. The method of claim 1, wherein the compound is capable of increasing a cell surface expression level of CD11a by MSCs.

15. The method of claim 1, wherein the effective amount of the compound composition is a concentration from about 0.01 micromolar to about 10 micromolar, wherein optionally the concentration is from about 0.1 micromolar to about 3 micromolar.

16. A method of improving a homing function of mesenchymal stem cells (MSCs), the method comprising the steps of:
(a) providing MSCs; and
(b) contacting the MSCs with a composition of claim 25, wherein the compound is capable of improving a homing function of MSCs;
wherein the homing function is one or more of
(i) increased expression of a cell surface molecule capable of facilitating a homing function, wherein optionally the cell surface molecule is CD11a,
(ii) increased in vitro adhesion by the MSCs in a shear flow assay,
(iii) increased binding of E-selectin or ICAM-1, and
(iv) increased homing and/or anti-inflammatory activity of the MSCs upon in vivo systemic administration of the MSCs in an animal inflammation model.

17. (canceled)

18. A composition comprising purified treated MSCs, wherein the purified treated MSCs are produced by the method of claim 16.

19. A pharmaceutical composition comprising an effective amount of purified treated MSCs, wherein the purified treated MSCs are produced by the method of claim 16, and a pharmaceutical carrier.

20. The pharmaceutical composition of claim 19, wherein the purified treated MSCs comprise pharmaceutical agents comprising therapeutic molecules, wherein the therapeutic molecules optionally comprise proteins.

21. A composition comprising a combination of purified MSCs in vitro and an effective amount of a composition of claim 25.

22. A method of screening to identify a small molecule compound capable of improving a homing function of MSCs, comprising the steps of:
(a) providing a candidate composition comprising a candidate small molecule compound;
(b) providing MSCs;
(c) treating the MSCs with the candidate composition, thereby generating treated MSCs;
(d) measuring a characteristic of the treated MSCs, wherein the characteristic comprises one or more of in vitro expression of a cell surface molecule capable of facilitating a homing function, in vitro adhesion of the treated MSCs in a shear flow assay, and anti-inflammatory activity upon in vivo systemic administration in an animal inflammation model;
(e) comparing one or more of the characteristics of treated MSCs relative to a characteristic of negative control MSCs, wherein the negative control MSCs are untreated or treated with a negative control candidate composition which is not capable of improving a homing function of MSCs; and
(f) identifying the small molecule compound capable of improving a homing function of MSCs wherein the small molecule compound, for treated MSCs relative to negative control MSCs, demonstrates one or more of increased expression of a cell surface molecule capable of facilitating a homing function, increased in vitro adhesion in a shear flow assay, reduced autoimmune disease activity upon in vivo systemic administration in an animal model, and increased anti-inflammatory activity upon in vivo systemic administration in an animal inflammation model.

23. The method of claim 22, wherein the cell surface molecule comprises CD11a, wherein the shear flow assay uses an E-selectin coated substrate, and the animal inflammation model is a mouse inflamed ear model.

24. The method of claim 22, wherein the shear flow assay of step (d) is an in vitro firm adhesion assay comprising the steps of:
(d-a) providing an assay plate comprising multiple wells wherein a microfluidic channel connects each pair of adjacent inlet and outlet wells;
(d-b) placing the assay plate under vacuum;
(d-c) coating the channels with recombinant human E-selectin or ICAM-1 and incubating for a time to allow sufficient coating;
(d-d) washing the wells;
(d-e) introducing compound-pretreated MSCs into the channel and allowing a time period for attachment without a flow being applied;
(d-f) subjecting putatively attached cells to increasing shear flow, optionally ranging from about 0.25 dynes/cm2 to about up to 10 dynes/cm2;
(d-g) obtaining data from observation of firmly adhered cells, optionally from acquired image data.

25. A pharmaceutical composition comprising an effective amount of a compound, wherein the compound is capable of improving a homing function of MSCs wherein the compound has structure of formula I: 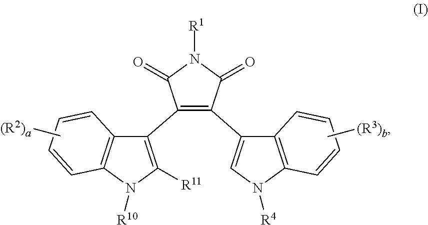
wherein
R¹ is selected from the series consisting of hydrogen, (C₁-C₄)-alkyl, phenyl-(C₁-C₄)-alkyl- and heteroaryl-(C₁-C₄)-alkyl-, wherein phenyl and heteroaryl are unsubstituted or substituted by substituents from the series consisting of halogen, (C₁-C₄)-alkyl and (C₁-C₄)-alkyl-O—;
R² and R³ are independently of each other selected from the series consisting of halogen, (C₁-C₄)-alkyl and (C₁-C₄)-alkyl-O—;
R⁴ is selected from the series consisting of hydrogen and (C₁-C₄)-alkyl,
R¹⁰ is selected from the series consisting of hydrogen, (C₁-C₆)-alkyl and Het¹, wherein (C₁-C₆)-alkyl is unsubstituted or substituted by R²⁰, and wherein Het¹ is unsubstituted or substituted by R²¹,
R¹¹ is selected from the series consisting of hydrogen and (C₁-C₄)-alkyl,
or R¹⁰ and together are a divalent group selected from the series consisting of the groups —(CH₂)ₘ—C(R²²)(R²³)—(CH₂)ₙ— and —(CH₂)ₘ—N(R²⁴)—(CH₂)ₙ—, wherein the moieties (CH₂)ₘ and (CH₂)ₙ are unsubstituted or substituted by (C₁-C₄)-alkyl,
R²⁰ is selected from the series consisting of R³⁰—O—, R³¹—N(R³²)—, H₂N—C(═NH)—S—, pyridinyl and Het², wherein Het² is unsubstituted or substituted by R³³;
R²¹ is selected from the series consisting of (C₁-C₄)-alkyl, phenyl-(C₁-C₄)-alkyl- and pyridinyl-(C₁-C₄)-alkyl-, wherein phenyl and pyridinyl are unsubstituted or substituted by substituents from the series consisting of halogen, (C₁-C₄)-alkyl and (C₁-C₄)-alkyl-O—;
R²² is selected from the series consisting of hydrogen and (C₁-C₄)-alkyl;
R²³ is selected from the series consisting of hydrogen, (C₁-C₄)-alkyl, R³¹—N(R³²)— and R³¹—N(R³²)—(C₁-C₄)-alkyl-;
R²⁴ is selected from the series consisting of hydrogen, (C₁-C₄)-alkyl, pyridinyl-(C₁-C₄)-alkyl- and R³¹—N(R³²)—(C₁-C₄)-alkyl-;
R³⁰, R³¹ and R³² are independently of each other selected from the series consisting of hydrogen and (C₁-C₄)-alkyl;
R³³ is selected from the series consisting of (C₁-C₄)-alkyl;
Het¹ is a 4-membered to 7-membered, monocyclic, saturated heterocycle comprising one ring nitrogen atom, which is bonded via a ring carbon atom;
Het² is a 4-membered to 7-membered, monocyclic, saturated heterocycle comprising one or two ring nitrogen atoms, which is bonded via a ring carbon atom or a ring nitrogen atom;
heteroaryl is a 5-membered or 6-membered, monocyclic, aromatic heterocycle comprising one or two identical or different ring heteroatoms selected from the series consisting of N, O and S;
a and b are independently of each other selected from the series consisting of 0, 1 and 2; m and n are independently of each other selected from the series consisting of 1 and 2 and a pharmaceutical carrier.

26. (canceled)

27. The method of claim 1, further comprising the steps before step (c) of:
(b″) freezing the pretreated MSCs, thereby generating frozen pretreated MSCs, and
(b′″) thawing the frozen pretreated MSCs.

28. A chilled or frozen composition of purified pretreated MSCs of claim 18, wherein the purified pretreated MSCs have been previously subject to pretreatment with an effective amount of the composition of claim 25 and are capable of an enhanced homing function relative to untreated MSCs, and wherein the chilled or frozen composition has a temperature of equal to or lower than 4° C., equal to or lower than −20° C., or equal to or lower than −80° C.; wherein the chilled or frozen composition optionally comprises a cryoprotectant.

29. A method of manufacturing a therapeutic composition of pretreated MSCs capable of an enhanced homing function, comprising the steps of:
(a) providing a population of MSCs;
(b) pretreating the MSCs by contacting the MSCs with a composition of claim 25, and wherein the homing function is one or more of
(i) increased expression of a cell surface molecule capable of facilitating a homing function, wherein optionally the cell surface molecule is CD11a,
(ii) increased in vitro adhesion by the MSCs in a shear flow assay,
(iii) increased binding of E-selectin or ICAM-1, and
(iv) increased homing and/or anti-inflammatory activity of the MSCs upon in vivo systemic administration of the MSCs in an animal inflammation model,
(c) optionally preparing a single dose aliquot or multi-dose aliquot of the composition of pretreated MSCs; and
(d) optionally freezing the pretreated MSCs, thereby generating frozen pretreated MSCs;
thereby generating the therapeutic composition of pretreated MSCs.

30. A composition comprising purified MSCs of claim 29 in vitro, wherein the MSCs are pretreated with a compound of formula I or any of formulas I-1 to I-19, and wherein the MSCs express an increased level of one or more of: (a) cell surface expression of CD11a, (b) binding activity to ICAM-1, and (c) binding activity to E-selectin; wherein the increased level is relative to a corresponding level for control MSCs, wherein the control MSCs are optionally untreated or stimulated with a negative control compound.
